(19) United States
(12) Patent Application Publication
(10) Pub. No.: US 2024/0209070 A1
(43) Pub. Date: Jun. 27, 2024

(54) COTH3 BINDING AGENTS AND USES THEREOF

(71) Applicant: LUNDQUIST INSTITUTE FOR BIOMEDICAL INNOVATION AT HARBOR-UCLA MEDICAL CENTER, Torrance, CA (US)

(72) Inventor: Ashraf S. IBRAHIM, Irvine, CA (US)

(21) Appl. No.: 18/558,138

(22) PCT Filed: May 6, 2022
(86) PCT No.: PCT/US2022/028206

§ 371 Date: Oct. 30, 2023

Related U.S. Application Data
(60) Provisional application No. 63/185,356, filed on May 6, 2021.

(51) Int. Cl. C07K 16/14; A61K 31/496; A61K 31/7048; A61K 39/395

(52) U.S. Cl. CPC C07K 16/14; A61K 31/496; A61K 31/7048; A61K 39/39575; C07K 2317/24; C07K 2317/92

(57) ABSTRACT

Presented herein, in certain embodiments, are binding agents that specifically bind to CotH3 and uses thereof.

### RELATED APPLICATIONS

This patent application claims the benefit of priority to U.S. Provisional Patent Application No. 63/185,356, filed May 6, 2021. The entire contents of the foregoing application are incorporated herein by reference in their entirety, including all text, tables, sequence listing and drawings.

### GOVERNMENT SUPPORT

This invention was made with government support under R01 AI063503 to the Lundquist Institute and R43 AI138904 to Vitalex Biosciences (a spinoff company of the Lundquist Institute) awarded by the National Institutes of Allergy and Infectious Diseases (NIAID). The government has certain rights in the invention.

### SEQUENCE LISTING

The instant application contains a Sequence Listing which has been submitted in ASCII format via EFS-Web and is hereby incorporated by reference in its entirety. Said ASCII copy, created on May 6, 2022, is named 022098-0568745_Sequence_Listing.txt and is 85,683 bytes in size.

### INTRODUCTION

The present invention relates generally to monoclonal binding agents (e.g., antibodies) that bind specifically to CotH3 and methods for detecting, treating and preventing infectious diseases in a subject. In certain embodiments, binding agents disclosed herein are used to treat or prevent mucormycosis.

About 180 of the 250,000 known fungal species are recognized to cause disease (mycosis) in man and animal. Some fungi can establish an infection in all exposed subjects, e.g., the systemic pathogens Histoplasma capsulatum and Coccidioides immitis. Others, such as Candida, Aspergillus and Zygomycetes species are opportunistic pathogens which typically cause disease in an immune-compromised host. Fungi of the class Zygomycetes, order Mucorales, can cause Mucormycosis, a deadly fungal infection in humans. Fungi belonging to the order Mucorales are distributed into at least six families, all of which can cause mucormycosis. However, fungi belonging to the family Mucoraceae (e.g., species belonging to Rhizopus, Mucor, and Lichtheimia) are by far the most common cause of infection. Increasing cases of mucormycosis have also been reported due to infection with Cunninghamella spp. in the Cunninghamellaceae family. The remaining four families of the Mucorales order cause disease less frequently.

Recent reports have demonstrated a striking increase in the number of reported cases of mucormycosis over the last two decades. There has also been an alarming rise in the incidence of mucormycosis at major transplant centers. For example, the Fred Hutchinson Cancer Center has reported a greater than doubling in the number of cases from 1985-1989 to 1995-1999. Also, the incidence of mucormycosis in transplant subjects has more than doubled over a similar time-span. Given the increasing prevalence of diabetes, cancer, and organ transplantation in the aging United States population, the rise in incidence of mucormycosis is anticipated to continue unabated for the foreseeable future until compounds and treatments are developed to mitigate mucormycosis and related fungal infections.

### SUMMARY

In some aspects, presented herein is a binding agent that specifically binds to CotH3, or a portion thereof. In certain embodiments, a binding agent specifically binds to a variant or homologue of CotH3.

In some aspects, presented herein is a CotH3 binding agent comprising a CDR-L1, CDR-L2 and CDR-L3 selected from Table 1, and a CDR-H1, CDR-H2 and CDR-H3 selected from Table 3. In some embodiments a CotH3 binding agent comprises a light chain variable region having at least 80% identity to a light chain variable region selected from Table 2 and a heavy chain variable region having at least 80% identity to a heavy chain variable region selected from Table 4. In some embodiments a CotH3 binding agent comprises a full-length light chain sequence having at least 80% identity to a full-length light chain sequence selected from Table 2 and a full-length heavy chain sequence having at least 80% identity to a full length heavy chain sequence selected from Table 4.

Certain aspects of the technology are described further in the following description, examples, claims and drawings.

### BRIEF DESCRIPTION OF THE DRAWINGS

The drawings illustrate embodiments of the technology and are not limiting. For clarity and ease of illustration, the drawings are not made to scale and, in some instances, various aspects may be shown exaggerated or enlarged to facilitate an understanding of particular embodiments.

FIG. 1 shows SDS-PAGE analysis of the purified chimeras and grafted humanized variants of Example 1. 10 μg of purified recombinant antibody was resolved by SDS-PAGE under non-reducing (lanes 1) or reducing (lanes 2) conditions and the gel stained with Coomassie Brilliant Blue and de-stained. The preparations appeared to be >95% purity.

FIG. 2A-2D shows binding traces obtained for on rates (measured between 0 and 300 seconds) and off rates (measured after washing the probe in LifeProbe's kinetics buffer at 300 seconds). Note the scale of the response (Y-axis) for the grafted clone is 0-0.1, compared to 0.7 or 1.2.

FIG. 3 shows models of the light chain variable kappa region (“Light chain”; teal blue=framework region, magenta=CDRs) and heavy chain variable region (“Heavy chain”; green=framework regions, brown=CDR) of a humanized grafted antibody of a 4LKX PDB carrying the grafted CDRs from the parental mouse C2 monoclonal antibody. Yellow regions shown in the right panel show potential regions of conflict with some of the key candidate residues for back-mutation highlighted.

FIG. 4 shows binding activity in real time of 30 nM CotH3 added to the indicated antibody clones captured by Protein G coated sensors recorded by a Gator system. Association was monitored for 300 seconds, then the sensors washed in binding buffer and the signal recorded for another 300 seconds to monitor CotH3 dissociation.

FIG. 5 shows binding traces for antigen association and dissociation at different antigen concentrations of the chimera and the LC3:HC3, LC3:HC2, LC2:HC3 humanized variants.

FIG. 6 shows SDS-PAGE analysis of the three active humanized antibody clones. 10 μg of purified humanized antibody was resolved by SDS-PAGE, stained with Coomassie Brilliant Blue and de-stained. Lanes 1, 3 and 5: ran under non-reducing conditions. Lanes 2, 4 and 6 ran under reducing conditions.

FIG. 7 shows Binding traces for antigen association and dissociation at different antigen concentrations of the chimera and the LC3:HC3, LC3:HC2 and LC2:HC3 humanized variants.

FIG. 8 shows steady state analysis of the KD values for the humanized variants compared to the wild-type chimera.

FIG. 9 shows steady state analysis of the KD values for the humanized variants compared to the wild-type chimera.

FIG. 10 shows anti-CotH3 humanized VX01 (LC3HC3) binding with CotH3 expressed on the cell surface of Yeast S. cevevisiae.

FIG. 11 shows lung tissues harvested from mice that have been intratraceally infected with Rhizopus delemar and treated 24 h later with a single dose of 30 μg of either an isotype-matched IgG (placebo), the murine anti-CotH3 C2 clone or the humanized LC3:HC3 (VX01) clone. Lungs were harvested on day 4 post infection. Lungs from Placebo showed fungal hyphae with extensive inflammation and tissue edema, while sections from mice infected and treated with either C2 or VX01 showed no fungal elements and normal lung architecture.

FIG. 12 shows a pharmacokinetic study of anti-CotH3 humanized VX01 (LC3HC3) and Mouse C2 antibody introduced intravenously in mice. Dose dependent pharmacokinetics are noticed.

FIG. 13 shows the Vitalex humanization: Round 1 Back-Mutations. Figure discloses SEQ ID NOS 49-56, respectively, in order of appearance, from top to bottom.

FIG. 14 shows steady state analysis of the binding affinity to rCotH3p at different antigen concentrations of the chimera and the HC3:LC3, LC3:HC2, LC2:HC3 humanized variants.

FIG. 15 shows the affinity measured in KD values for the back mutation variant, mouse C2 mAb and the chimera mAb.

FIG. 16 shows binding affinity of humanized anti-CotH3 mAbs.

FIG. 17 shows the specificity/toxicity test results of the humanized CotH3 LC2:HC3, LC3:HC2, LC3:HC3 variants by Western Blot.

FIG. 18 shows the specificity/toxicity test results of the humanized CotH3 LC3:HC2, LC3:HC3 variants by Western Blot.

FIG. 19 shows the flow cytometric analyses of humanized antibody binding with Mucorales species (Rhizopus delemar, Cunninghamella bertholletiae, Lichtheimia corymbifera).

FIG. 20 shows the flow cytometric analyses of humanized antibody binding with Mucorales species (Mucor circinelloides, Rhizomucor).

FIG. 21 shows the ability of LC3:HC3's enhanced neutrophil killing activity of Rhizopus.

FIG. 22 shows that humanized antibodies are as protective as Murine C2 mAb in the Neuropenic mouse model of Mucormycosis caused by R. delemar 99-880 infection.

FIG. 23 shows that humanized antibodies are as protective as Murine C2 mAb in the Neuropenic mouse model of Mucormycosis caused by R. delemar 99-880 infection (combined data).

FIG. 24 shows that humanized antibodies are as protective as Murine C2 mAb in the Neuropenic mouse model of Mucormycosis caused by R. delemar 99-880 infection (cfu data).

FIG. 25 shows that humanized antibodies are as protective as Murine C2 mAb in the Neuropenic mouse model of Mucormycosis caused by Mucor circinelloides infection.

FIG. 26 shows that humanized CotH3 antibody (VX01) acts synergistically with antifungal agents in a severe infection model of R. delemar.

FIG. 27 shows the reduction of mouse tissue fungal burden in lungs and brain after combination treatment with Posaconazole and humanized CotH3 antibody (VX01).

FIG. 28 shows the reduction of mouse tissue fungal burden in lungs and brain after combination treatment with LAmB (liposomal amphotericin B) and humanized CotH3 antibody (VX01).

FIG. 29 shows that humanized CotH3 antibody (VX01) acts synergistically with antifungal agents in a severe infection model of M. circinelloides.

### DETAILED DESCRIPTION

Presented herein, in some embodiments, are binding agents that bind specifically to CotH3, or a portion thereof, as well as compositions and uses thereof. In some embodiments binding agents presented herein are used for the treatment, prevention and/or diagnosis of mucormycosis in a subject.

Mucorales CotH proteins are cell surface proteins expressed by fungi in the Mucorales order that are involved in the process of adherence and invasion of host cells, such as endothelial cells. A mammalian homologue of CotH proteins have not been identified. Therefore, the presence, absence and/or amount of a CotH polypeptide in a subject can serve as an indicator of the presence, absence or severity of infection by a Mucorales species.

A CotH3 protein may be a CotH3 of any Mucorales species. In some embodiments a CotH3 protein comprises a polypeptide sequence set forth in any one of SEQ ID NOS:35-37.

### Subjects

The term “subject” refers to animals, typically mammalian animals. Any suitable mammal can be treated by a method or composition described herein. Non-limiting examples of mammals include humans, non-human primates (e.g., apes, gibbons, chimpanzees, orangutans, monkeys, macaques, and the like), domestic animals (e.g., dogs and cats), farm animals (e.g., horses, cows, goats, sheep, pigs) and experimental animals (e.g., mouse, rat, rabbit, guinea pig). In some embodiments a mammal is a human. A mammal can be any age or at any stage of development (e.g., an adult, teen, child, infant, or a mammal in utero). A mammal can be male or female. A mammal can be a pregnant female. In certain embodiments a mammal can be an animal disease model, for example, animal models used for the study of fungal infections.

In some embodiments a subject has a Mucorales infection or is suspected of having a Mucorales infection. In some embodiments a subject suspected of having a Mucorales infection shows physiologic signs and/or symptoms associated with a Mucorales infection. In some embodiments a medical professional (e.g., a physician) determines that a subject is suspected of having a Mucorales infection. In some embodiments a subject or mammal is “at risk” of acquiring a Mucorales infection. A mammal that is at risk may have increased risk factors for acquiring a fungal infection, non-limiting examples of which include immunocompromised individuals or immune deficient subjects (e.g., bone marrow transplant recipients, irradiated individuals, subjects having certain types of cancers, particularly those of the bone marrow and blood cells (e.g., leukemia, lymphoma, multiple myeloma), subjects with certain types of chronic infections (e.g., HIV, AIDS), subjects treated with immunosuppressive agents, subjects suffering from malnutrition and aging, subjects taking certain medications (e.g. disease-modifying anti-rheumatic drugs, immunosuppressive drugs, glucocorticoids), subjects undergoing chemotherapy, the like or combinations thereof). In some embodiments a subject at risk is, will be, or has been in a location or environment suspected of containing a Mucorales species (e.g., a Mucorales pathogen, e.g., spores of a Mucorales pathogen). For example, a subject at risk can be a medical professional that is providing care to another who is suspected of being infected with, or known to be infected with Mucorales. In certain embodiments, a subject at risk is any subject that has been exposed to Mucorales. In certain embodiments, a subject at risk is any patient who is, will be, or has been in a hospital or medical care facility suspected of containing Mucorales. In certain embodiments, a subject at risk is any patient who is, will be, or has recently been (e.g., within 1 day to 1 year, or within 3 months to 6 months), in an intensive care unit, long term acute care hospital, rehabilitation hospital or facility, or skilled nursing facility. In certain embodiments, a subject at risk is on mechanical ventilation. In certain embodiments, a subject at risk is any patient who has, will have, or has had a central venous catheter, including a peripherally inserted central catheter. In certain embodiments, a subject at risk is on mechanical ventilation. In certain embodiments, a subject at risk is any patient who has undergone an invasive medical treatment or procedure.

In some embodiments a subject in need of a treatment or composition described herein is a subject at risk of a Mucorales infection and/or a subject that has a Mucorales infection. In some embodiments a subject in need of a treatment or composition described herein is infected with, or is suspected of being infected with Mucorales. In certain embodiments an antibody binding agent (e.g., an antibody or the like) or composition described herein is used to treat or prevent a Mucorales infection in a subject or a subject at risk of acquiring a Mucorales infection.

In some embodiments a subject in need of a treatment or composition described herein is a donor. In some embodiments a donor is healthy subject or a moderately healthy subject. In some embodiments a donor is free of a Mucorales infection. A donor may or may not be at risk of acquiring a Mucorales infection. In some embodiments a donor is an organ donor. In some embodiments a donor is preselected or predetermined to donate an organ, blood, bone marrow, serum, or the like to a subject who is at risk, or will become at risk of acquiring a Mucorales infection. Thus a donor is sometimes a subject in need of a treatment or a composition described herein.

### Mucorales

In some embodiments method are described herein for detecting, diagnosing, preventing and/or treating Mucorales (e.g., the presence or absence of a Mucorales species) or a Mucorales infection (e.g., Mucormycosis). In some embodiments Mucorales refers to any pathogenic or potentially pathogenic strain, species or isolate of Mucorales capable of causing an infection in a subject. A Mucorales infection refers to the presence of any pathogenic or potentially pathogenic strain, species or isolate of Mucorales in a subject (e.g., a mammalian subject, e.g., a human). In some embodiments Mucorales refers to a strain or isolate of Mucorales that displays resistance to one or more drugs (e.g., anti-fungal drugs) or anti-fungal treatments. In certain embodiments Mucorales is a Mucorales species, strain or isolate that is resistant to multiple drugs (e.g., a multi-drug resistant strain).

A Mucorales infection can be detected, prevented or treated by a method or use of a binding agent or composition described herein. Mucorales infections can be systemic and/or local. Non-limiting examples of local Mucorales infections include infections of the skin (epidermis, dermis, hypodermis, subcutaneous tissue), epithelial membranes, sinus membranes, ears, eyes, nose, throat, mouth, scalp, feet, nails, vagina, endometrium, urinary tract (e.g., bladder, urethra), the like, portions thereof or combinations thereof. Non-limiting examples systemic Mucorales infections include infection of one or more tissues or organs, non-limiting examples of which include liver, kidney, heart, muscle, lung, stomach, large intestine, small intestine, testis, ovaries, brain, nervous tissue, blood, lymph, lymph nodes, salivary glands, the like or combinations thereof.

Non-limiting examples of Mucorales species include A. idahoensis, A. corymbifera, Apophysomyces elegans, Actinomucor elegans, A. rouxii, B. circina, B. multispora, C. brefeldii, C. angarensis, Cunninghamella bertholletiae (C. bertholletiae), Choanephora cucurbitarum, C. recurvatus, D. fulva, E. anomalus, H. elegans, H. assamensis, K. cordensis, Lichtheimia corymbifera (L. corymbifera), Lichtheimia ramosa, M. ambiguus, Mucor amphibiorum, Mucor circinelloides, M. verticillata, Parasitella parasitica, P. agaricine, P. anomala, P. circinans, Phycomyces blakesleeanus, S. umbellata, S. megalocarpus, T. elegans, T. indicae-seudaticae, Z. californiensis, Rhizomucor endophyticus, Rhizopus javensis, R. azygosporus, Rhizopus caespitosus, Rhizopus homothallicus, Rhizopus oryzae, Rhizopus stolonifer, Rhizopus reflexus, Rhizopus microsporus (e.g., var. rhizopodiformis), and Rhizopus schipperae. The species “R. oryzae” as referenced herein is used synonymously with the species “R. delemar”, as R. oryzae is the same species as R. delemar. The species Rhizopus oryzae was renamed as Rhizopus delemar in 2007 (e.g., see Abe A. et al. (2007) Mycologia 99(5):714-722). Rhizopus delemar is sometimes referred to as Rhizopus delamari in the literature. Non-limiting examples of strains of R. oryzae include R. oryzae 99-880, R. oryzae 99-892 and R. oryzae 21477. In certain embodiments, a Mucorales species is a species that expresses a CotH3 polypeptide. In certain embodiments, a Mucorales species is a species that expresses a CotH3 polypeptide comprising the sequence set forth in SEQ ID NO:34.

### Binding Agents

In certain embodiments, a binding agent comprises or consists of one or more antigen binding portions derived from an antibody that bind specifically to a CotH3 protein, or a portion thereof. An antigen binding portion of a binding agent or antibody is that portion that binds specifically to an antigen. A binding agent may comprise one antigen binding portion or multiple antigen binding portions. In some embodiments a binding agent comprises at least one antigen binding portion (i.e. a binding portion). In some embodiments a binding agent comprises 1, 2, 3, 4, 5, 6, 7, 8, 9, or 10 or more binding portions. In some embodiments a binding agent comprises one antigen binding portion (i.e. a monovalent binding antigen), two antigen binding portions (divalent) or three antigen binding portions (trivalent). In certain embodiments, all of the binding portions of a multivalent binding agent bind to the same antigen. In certain embodiments, all of the binding portions of a multivalent binding agent comprise one or more polypeptide sequences that are at least 90%, at least 95%, at least 99% or 100% identical.

In some embodiments a binding agent comprises an antibody, or a portion thereof (e.g., an antigen binding portion thereof). In certain embodiments, a binding agent comprises or consists of an antibody, an antibody fragment and/or an antigen binding portion of an antibody (e.g., a binding fragment, i.e., a binding portion thereof). In some embodiments a binding agent is an antibody (e.g., a monoclonal antibody and/or a recombinant antibody). A binding agent or antibody can be generated, manufactured or produced by a suitable method. In some embodiments a binding agent is monoclonal. In some embodiments a binding agent is a monoclonal antibody derived from a suitable species. Certain non-limiting examples of a binding agent include monoclonal antibodies, chimeric antibodies, antibody binding fragments (e.g., an antigen binding portion of an antibody), a CDR-grafted antibody, a humanized antibody, and a human antibody, or portions thereof. Human antibodies can be obtained by any suitable method. For example, human antibodies can be obtained from trans-chromosomal animals engineered to produce fully human antibodies. In some embodiments, a human antibody is obtained from screening one or more expression libraries comprising nucleic acids that encode fully human antibodies, human antibody heavy chains, human antibody light chains, human heavy chain variable regions, human light chain variable regions, and/or combinations thereof. Nucleic acids encoding a binding agent, or a portion thereof can be subcloned into a suitable expression vector, and expressed in a suitable expression system. In certain embodiments, a binding agent is not polyclonal, and/or is not a polyclonal antibody.

In some embodiments a binding agent is derived, produced, obtained, isolated, and/or purified from a suitable species. In some embodiments a binding agent is derived, produced, obtained, isolated, and/or purified from a rabbit, goat, horse, cow, rat, mouse, fish, bird, or llama, for example. In some embodiments a binding agent is derived, produced, obtained, isolated, and/or purified from a bird (e.g., a chicken, or a bird egg). In some embodiments a binding agent is derived, produced, obtained, isolated, and/or purified from a plant (e.g., a recombinant binding agent produced by a genetically engineered plant). In some embodiments a binding agent is derived, produced, obtained, isolated, and/or purified from a suitable mammal. In certain embodiments a suitable mammal is a genetically altered mammal (e.g., a trans-chromosomal or transgenic mammal) engineered to produce antibodies comprising human heavy chains and/or human light chains or portions thereof. In some embodiments a binding agent is produced, obtained, isolated, or purified from a prokaryotic or eukaryotic cell (e.g., a recombinant binding agent produced by a genetically engineered cell). In some embodiments a binding agent is produced, obtained, isolated, or purified from a virus (e.g., a recombinant binding agent produced by a genetically engineered virus).

A binding agent can be expressed, isolated from and/or purified from a suitable expression system non-limiting examples of which include a suitable bacteria, phage, insect, virus, plant or mammalian expression system. For example, a nucleic acid encoding a binding agent can be introduced into a suitable mammalian cell line that expresses and secretes the binding agent into the cell culture media. Any suitable mammalian cell line can be used. In certain embodiments a mammalian cell line is a Chinese hamster ovary (CHO) cell line. A method of producing a binding agent (e.g. CotH3 binding agent) may comprise one or more of (i) introducing one or more nucleic acids into a suitable cell line wherein the nucleic acid directs the expression of a binding agent; (ii) culturing the cell line using a suitable culturing method for a period of time that allows expression of the binding agent; (iii) harvesting the cell line (e.g., by way of generating a lysate) or harvesting conditioned media from the cell line (e.g., where the binding agent is secreted); and (iv) isolating and/or purifying the binding agent using a suitable method.

In certain embodiments, a monoclonal antibody or a monoclonal binding agent is a substantially homogeneous population of binding agents, or binding fragments thereof, where each individual binding agent in the population is substantially identical and/or binds to the same epitope, with the exception of possible variants that may arise during production of a monoclonal binding agent. In some embodiments such variants generally are absent or may be present in minor amounts. In contrast to polyclonal antibody preparations which typically include a population of different antibodies directed against different determinants (epitopes) of an antigen, each binding agent of a population of monoclonal binding agents often binds a single determinant on an antigen. Monoclonal binding agents are often not contaminated by other immunoglobulins. Although one or more different monoclonal binding agents may be purposely added to a composition to form a mixture.

The modifier “monoclonal” is not to be construed as requiring production of a binding agent by any particular method. A monoclonal binding agent can be produced by any suitable method. For example, in certain embodiments, a monoclonal antibody is made by the hybridoma method (e.g., as described by Kohler et al, Nature, 256:495 (1975)), or a variation thereof. In some embodiments a monoclonal binding agent is made by a recombinant DNA method. For example, a monoclonal binding agent can be made by screening a recombinant library using a suitable expression system (e.g., a phage display expression system). In some embodiments a monoclonal binding agent is isolated from a phage library of binding agents, for example by using a technique described in Clackson et al, Nature, 352:624-628 (1991) and/or Marks et al, J. Mol Biol, 222:581-597 (1991), or a variation thereof.

In certain embodiments, a binding agent comprises one or more structural or backbone portions, sometimes referred to as scaffolds. A binding agent may comprise a scaffold, non-limiting examples of which include a scaffold derived from an antibody, a Z domain of Protein A, gamma-B crystalline, ubiquitin, cystatin, Sac7d, a triple helix coiled coil, a lipocalin, an ankyrin repeat motif, a Kunitz domain of a suitable protease inhibitor, a fibronectin domain, a nucleic acid polymer, the like, portions thereof or combinations thereof. In some embodiments a binding agent does not comprise a scaffold. In certain embodiments, a binding agent comprises one or more structural portions of a mammalian antibody (e.g., a human antibody).

In certain embodiments a binding agent comprises one or more constant regions (e.g., constant regions derived from an antibody, e.g., a mammalian antibody). A binding agent may comprise any suitable constant region of an antibody, or one or more portions thereof. In certain embodiments a binding agent comprises a constant region of an antibody light chain and/or a constant region of an antibody heavy chain. In some embodiments a binding agent comprises a lambda (λ) light chain constant region, or a portion thereof. In some embodiments a binding agent comprises a kappa (κ) light chain constant region, or a portion thereof. In some embodiments a binding agent comprises a polypeptide that is at least 75%, at least 80%, at least 85%, at least 90%, at least 95% or at least 99% identical to a polypeptide sequence of a light chain constant region of a mammalian antibody, or portion thereof. In some embodiments a binding agent comprises a polypeptide that is at least 75%, at least 80%, at least 85%, at least 90%, at least 95% or at least 99% identical to a polypeptide sequence of a light chain constant region of a human antibody. In some embodiments a binding agent does not include a light chain constant region.

In certain embodiments a binding agent comprises a constant region of an antibody heavy chain. A binding agent can include any suitable heavy chain constant region, or portion thereof. In mammals, an antibody can have at least five types/classes of Ig heavy chains denoted as IgA, IgD, IgE, IgG, and IgM, which are determined by the presence of distinct heavy chain constant regions, or portion thereof (e.g., CH1, CL, CH2, CH3 domains). In some embodiments a binding agent comprises one or more heavy chain constant regions of an IgM, IgD, IgA, or IgE isotype, or a portion thereof. In some embodiments a binding agent comprises a heavy chain constant region of an IgG₁, IgG₂, IgG₃ or IgG₄, or one or more portions thereof. In some embodiments a binding agent comprises a polypeptide that is at least 75%, at least 80%, at least 85%, at least 90%, at least 95%, at least 99% identical, or 100% identical to a polypeptide sequence of a heavy chain constant region of a mammalian antibody. In some embodiments a binding agent comprises a polypeptide that is at least 75%, at least 80%, at least 85%, at least 90%, at least 95%, at least 99% identical or 100% identical to a polypeptide sequence of a heavy chain constant region of a human antibody. In some embodiments a binding agent comprises one or more additions, deletions and/or modification to a constant region. A binding agent is sometimes modified to change the antibody class, or isotype of a binding agent. In some embodiments a binding agent comprises one or more additions, deletions and/or modification (one or more amino acid substitutions, deletions or additions) to modify one or more functions of a binding agent, for example to abolish, enhance or decrease serum half-life, Fc receptor binding, complement binding (e.g., C1q binding), glycosylation, sialylation, cellular toxicity, antibody-dependent cell-mediated phagocytosis (ADCP), antibody dependent cellular cytotoxicity (ADCC), and the like. In some embodiments a binding agent does not include one or more portions of a heavy chain constant region or light chain constant region. In some embodiments a binding agent does not include a heavy chain constant region.

In some embodiments a binding agent comprises or consists of one or more variable regions of an antibody, or a portion thereof. In some embodiments a binding agent comprises one or more light chain variable regions, or a portion thereof. In some embodiments a binding agent comprises one or more heavy chain variable regions, or a portion thereof. In certain embodiments a binding agent comprises at least one light chain variable region and at least one heavy chain variable region. A light chain variable region and heavy chain variable region can be on the same or different polypeptides.

In certain embodiment, a binding agent is a non-naturally occurring binding agent. Non-limiting examples of non-naturally occurring binding agents include monoclonal binding agents (e.g., monoclonal antibodies), chimeric antibodies, CDR-grafted antibodies, humanized antibodies, single-chain antibodies, Fab, Fab′, single chain Fab (scFab), F(ab′)2, Fv fragment, single-chain Fv (scFv), scFv-Fc, (scFv)2-Fc, disulfide-linked Fvs (sdFv), VL, VH, diabody (Dab), triabody (trivalent), tetrabody (tetravalent), minibody ((scFV-CH3)2), IgGdeltaCH2, synbody, fynomers, affibodies, affilins, affimers, affitins, alphabodies, anticalins, avimers, DARPins, Kunitz domain peptides, monobodies, TandAbs, nanobodies, BiTEs, SMIPs, DNLs, Duocalins, adnectins, Albu-dabs, DARTs, DVD-IG, Covx-bodies, peptibodies, scFv-Igs, SVD-Igs, dAb-Igs, Knob-in-Holes, triomAbs, the like, combinations thereof, and antigen binding portions thereof.

In some embodiments a binding agent comprises or consists of a Fab, scFab, Fab′, F(ab′)2, Fv fragment, single-chain Fv (scFv), diabody (Dab), synbody, the like and/or a combination or portion thereof. In some embodiments a binding agent is a Fab, Fab′, F(ab′)2, Fv fragment, single-chain Fv (scFv), diabody (Dab), synbody, the like and/or a combination, or portion thereof (see, e.g., U.S. Pat. Nos. 6,099,842 and 5,990,296). In some embodiments a binding agent comprises a single-chain polypeptide comprising one or more antigen binding portions. For example, a single-chain binding agent can be constructed by joining a heavy chain variable region, or antigen binding portion thereof, with a light chain variable region, or antigen binding portion thereof, with a linker (e.g., an amino acid, a polypeptide linker) using recombinant molecular biology processes. Such single chain binding agents often exhibit specificities and affinities for an antigen similar to a parent two-chain monoclonal binding agent. Binding agents often comprise engineered regions such as CDR-grafted or humanized portions. In certain embodiments a binding agent is an intact two-chain immunoglobulin, and in other embodiments a binding agent is a Fab monomer or a Fab dimer.

Nucleic acids, or portions thereof, that encode a polypeptide of a binding agent may be cloned, subcloned, rearranged or modified for recombinant expression by a suitable cloning procedure and subsequently expressed using a suitable expression system by a method known to those skilled in the art (e.g., see Maniatis et al., Molecular Cloning: A Laboratory Manual, Cold Spring Harbor Laboratory, 1982; Antibody Engineering: Methods and Protocols, Vol. 248 of Methods in molecular biology, edited by Benny K. C. Lo, Springer Science & Business Media, 2004; Antibody Engineering, Vol. 1, Roland E. Kontermann, Stefan Dubel, Edition 2, Publisher Springer Science & Business Media, 2010; Antibody Phage Display: Methods and Protocols, Biomed Protocols, Vol. 178 of Methods in molecular biology, Editors Philippa M. O'Brien, Robert Aitken, Springer Science & Business Media, 2004).

In mammals, the heavy chain variable region and light chain variable region of an antibody each contribute three CDRs (complementarity determining regions) commonly referred to as CDR1, CDR2 and CDR3, that are often separated and/or flanked by framework regions (e.g., FR1, FR2, FR3 and FR4). The term “CDR” as used herein refers to an amino acid sequence of a polypeptide identified as a complementarity determining region. In certain embodiments, definitive delineation of a CDR polypeptide sequence and identification of residues comprising the binding site of a binding agent is accomplished by solving the structure of a binding agent and/or solving the structure of a binding agent-antigen complex. In certain embodiments, this can be accomplished by any suitable method, such as X-ray crystallography and/or computer modeling. In certain embodiments, various methods of analysis can be employed to identify or approximate the CDR sequences of a binding agent or antibody. For example, the amino acid sequence and/or location of CDRs in a polypeptide sequence of a binding agent, an antibody, a binding portion thereof or variable region thereof, can be identified using a suitable method, non-limiting examples of which include the Kabat system (e.g., see Kabat, E. A., et al., 1991; Sequences of Proteins of Immunological Interest, Fifth Edition, NIH Publication No. 91-3242, as well as Johnson, G. and Wu, T. T. 2000, Nucleic Acids Research), and/or the Chothia Numbering Scheme (e.g., Chothia & Lesk, (1987) J. Mol. Biol, 196:901-917; Chothia et al, Nature, (1989) 342:878-883; and Al-Lazikani et al., (1997) JMB 273, 927-948). In some embodiments the amino sequence and/or location of CDRs of an antibody can be identified using the AbM method and/or contact method. The “AbM” definition uses an integrated suite of computer programs produced by Oxford Molecular Group that model antibody structure (see e.g., Martin et al, Proc. Natl. Acad. Sci. (USA), 86:9268-9272 (1989); “AbM™, A Computer Program for Modeling Variable Regions of Antibodies,” Oxford, UK; Oxford Molecular, Ltd.). The AbM definition models the tertiary structure of an antibody from primary sequence using a combination of knowledge databases and ab initio methods, such as those described by Samudrala et al., “Ab Initio Protein Structure Prediction Using a Combined Hierarchical Approach,” in PROTEINS, Structure, Function and Genetics Suppl, 3:194-198 (1999). In certain embodiments, a contact definition is based on an analysis of the available complex crystal structures (see e.g., MacCallum et ah, J. Mol. Biol, 5:732-45 (1996)).

In some embodiments a binding agent and/or an antigen binding portion of a binding agent comprises at least 2, at least 3, at least 4, at least 5 or at least 6 CDRs. In some embodiments a binding agent comprises 3 to 60 CDRs (e.g., for binding agents having multiple antigen binding portions). In some embodiments a binding agent comprises 3 to 12 CDRs. In some embodiments an antigen binding portion of a binding agent comprises 1 to 6 CDR polypeptide sequences.

In some embodiments a light chain variable region of a binding agent comprises one or more CDRs (e.g., one, two, three, or more CDRs). In certain embodiments, a binding agent and/or an antigen binding portion of a binding agent comprises one, two or three CDRs of a light chain variable region. The amino acid sequences representing a CDR in a light chain variable region of an antibody or binding agent is referred to as CDR-L1, CDR-L2, and CDR-L3 which are numbered sequentially (i.e., L1, L2 and L3) in the direction from the amino terminus (N-terminus) to the carboxy terminus (C-terminus) of a light chain variable region. For example, in a polypeptide representing a light chain variable region of a binding agent, CDR-L1, when present, is often the most N-terminal light chain CDR; CDR-L3, when present, is often the most C-terminal light chain CDR; and CDR-L2, when present, is often located (i) between CDR-L1 and CDR-L3, (ii) on the N-terminal side of CDR-L3 or (iii) on the C-terminal side of CDR-L1, of a light chain variable region or binding portion of a binding agent. The terms “CDR-L1”, “CDR-L2” and “CDR-L3” refer to, in part, an amino acid sequence of a polypeptide identified as, or disclosed herein as, a complementarity determining region of a binding agent (e.g., a CDR of a light chain variable region). Non-limiting examples of amino acid sequences of a CDR-L1, CDR-L2 and CDR-L3 are provided in Table 1. A light chain variable region or antigen binding portion of a binding agent described herein may comprise any combination of a CDR-L1, a CDR-L2, and a CDR-L3 disclosed herein, wherein the binding agent retains specific binding to CotH3, or a portion thereof.

In certain embodiments, a binding agent comprises at least one light chain CDR-L1 comprising an amino acid sequence at least 70%, at least 80%, at least 90%, at least 95%, or 100% identical to a CDR-L1 selected from Table 1. In certain embodiments, a binding agent comprises a single light chain CDR-L1 comprising an amino acid sequence at least 70%, at least 80%, at least 90%, at least 95%, or 100% identical to a CDR-L1 selected from Table 1. In certain embodiments, a light chain variable region or antigen binding portion of a binding agent described herein comprises an amino acid sequence at least 70%, at least 80%, at least 90%, at least 95%, or 100% identical to a CDR-L1 selected from Table 1, and any other suitable CDR-L2 and/or CDR-L3 polypeptide sequence (e.g., a CDR-L2 or CDR-L3 selected from Table 1), where the binding agent retains specific binding to CotH3, or a portion thereof.

In certain embodiments, a binding agent comprises at least one light chain CDR-L2 comprising an amino acid sequence at least 70%, at least 80%, at least 90%, at least 95%, or 100% identical to a CDR-L2 selected from Table 1. In certain embodiments, a binding agent comprises a single light chain CDR-L2 comprising an amino acid sequence at least 70%, at least 80%, at least 90%, at least 95%, or 100% identical to a CDR-L2 selected from Table 1. In certain embodiments, a light chain variable region or antigen binding portion of a binding agent described herein comprises an amino acid sequence at least 70%, at least 80%, at least 90%, at least 95%, or 100% identical to a CDR-L2 selected from Table 1, and any other suitable CDR-L1 and/or CDR-L3 polypeptide sequence (e.g., a CDR-L1 or CDR-L3 selected from Table 1), where the binding agent retains specific binding to CotH3, or a portion thereof.

In some embodiments a binding agent comprises one or more light chain CDRs that are at least 70%, 75%, 85%, 86%, 87%, 88%, 89%, 90%, 91%, 92%, 93%, 94%, 95%, 96%, 97%, 98%, or at least 99% identical to any one of the CDR sequences listed in Table 1. In certain embodiments, a binding agent comprises at least one light chain CDR-L3 comprising an amino acid sequence at least 70%, at least 80%, at least 90%, at least 95%, or 100% identical to a CDR-L3 selected from Table 1. In certain embodiments, a binding agent comprises a single light chain CDR-L3 comprising an amino acid sequence at least 70%, at least 80%, at least 90%, at least 95%, or 100% identical to a CDR-L3 selected from Table 1. In certain embodiments, a light chain variable region or antigen binding portion of a binding agent described herein comprises an amino acid sequence at least 70%, at least 80%, at least 90%, at least 95%, or 100% identical to a CDR-L3 selected from Table 1, and any other suitable CDR-L1 and/or CDR-L2 polypeptide sequence (e.g., a CDR-L1 or CDR-L2 selected from Table 1), where the binding agent retains specific binding to CotH3, or a portion thereof.

In certain embodiments, a binding agent comprises two light chain CDRs independently selected from a CDR-L1, CDR-L2, and CDR-L3 of Table 1, where the binding agent specifically binding to CotH3, or a portion thereof. In certain embodiments, a binding agent comprises two light chain CDRs independently selected from a CDR-L1, CDR-L2, and CDR-L3 of any one clone listed in Table 1, where the binding agent specifically binding to CotH3, or a portion thereof. In certain embodiments, a binding agent comprises a CDR-L1, CDR-L2, and CDR-L3 comprising or consisting of an amino acid sequence at least 70%, at least 80%, at least 90%, at least 95%, or 100% identical to a CDR-L1, CDR-L2, and CDR-L3 selected from any one clone of Table 1, where the binding agent specifically binding to CotH3, or a portion thereof. In certain embodiments, a binding agent comprises a CDR-L1, CDR-L2, and CDR-L3 selected from any one clone or type listed in Table 1.

TABLE 1
Light Chain CDR Sequences

| | | | CDR-L1 | | CDR-L2 | | CDR-L3 |
|---|---|---|---|---|---|---|---|
| Clone | | CDR-L1 | SEQ ID | CDR-L2 | SEQ ID | CDR-L3 | SEQ ID |
| Number | Type | Sequence | NO: | Sequence | NO: | Sequence | NO: |
| C2- | Murine | QSLLDSDG | 1 | LVS | 4 | WQGTHFPH | 6 |
| 6E11A8 | | KTFL | | | | T | |
| C2- | Murine | QSLLYSNG | 2 | QVS | 5 | LQGTYFPH | 7 |
| AdvantGen | | KTY | | | | T | |
| C2 | Chimera 1 | QSLLDSDG | 3 | LVS | 4 | WQGTHFPH | 6 |
| | Humanized | KTF | | | | T | |
| C2 | Chimera 2 | QSLLYSNG | 2 | QVS | 5 | LQGTYFPH | 7 |
| | Humanized | KTY | | | | T | |
| C2 | Grafted | QSLLDSDG | 3 | LVS | 4 | WQGTHFPH | 6 |
| | Humanized | KTF | | | | T | |

In some embodiments a CotH3 binding agent or the antigen binding portion of a CotH3 binding agent comprises a light chain variable region having an amino acid sequence at least 70%, 75%, 85%, 86%, 87%, 88%, 89%, 90%, 91%, 92%, 93%, 94%, 95%, 96%, 97%, 98%, or at least 99% identity to light chain variable region sequence of Table 2. In some embodiments a CotH3 binding agent or the antigen binding portion of a CotH3 binding agent comprises a light chain variable region comprising or consisting of an amino acid sequence of a light chain variable region selected from Table 2. In some embodiments a CotH3 binding agent or the antigen binding portion of a CotH3 binding agent comprises a light chain sequence (Full-length Sequence) selected from Table 2.

TABLE 2
Variable Light Chain Sequences

| | | | | | LC Full |
|---|---|---|---|---|---|
| | | Light Chain | LCVR | Light Chain | Length |
| Clone | | Variable | SEQ | (Full-length | SEQ ID |
| Number | Type | Region (LCVR) | ID NO: | Sequence) | NO: |
| C2- | Murine | DVVMTQTPLTLSVTIGQ | 40 | | |
| 6E11A8 | kappa | PASISCKSSQSLLDSDGK | | | |
| | | TFLNWLLQRPGQSPKRL | | | |
| | | IYLVSKLDSGVPDRFTG | | | |
| | | TGSGTDFTLKISRVEAE | | | |
| | | DLGVYYCWQGTHFPHT | | | |
| | | FGAGTKLELKR | | | |
| C2- | Murine | DVVMTQTPLSLSVTIGQ | 8 | | |
| AdvantGen | kappa | PASISCKSSQSLLYSNGK | | | |
| | | TYLNWLQQRPGQAPKL | | | |
| | | LMFQVSKPGIPDRFSGS | | | |
| | | GSETDFTLKISRVEAEDL | | | |
| | | GVYYCLQGTYFPHTFG | | | |
| | | AGTKVEIK | | | |
| C2 | Chimera 1 | DVVMTQTPLTLSVTIGQ | 9 | DVVMTQTPLTLSVTIGQ | 15 |
| | Humanized | PASISCKSSQSLLDSDGK | | PASISCKSSQSLLDSDGK | |
| | | TFLNWLLQRPGQSPKRL | | TFLNWLLQRPGQSPKRL | |
| | | IYLVSKSGVPDRFTGTG | | IYLVSKSGVPDRFTGTGS | |
| | | SGTDFTLKISRVEAEDL | | GTDFTLKISRVEAEDLG | |
| | | GVYYCWQGTHFPHTFG | | VYYCWQGTHFPHTFGA | |
| | | AGTKVEIK | | GTKVEIKRTVAAPSVFIF | |
| | | | | PPSDEQLKSGTASVVCL | |
| | | | | LNNFYPREAKVQWKVD | |
| | | | | NALQSGNSQESVTEQDS | |
| | | | | KDSTYSLSSTLTLSKAD | |
| | | | | YEKHKVYACEVTHQGL | |
| | | | | SSPVTKSFNRGEC | |
| C2 | Chimera 2 | DVVMTQTPLSLSVTIGQ | 10 | DVVMTQTPLSLSVTIGQ | 16 |
| | Humanized | PASISCKSSQSLLYSNGK | | PASISCKSSQSLLYSNGK | |
| | | TYLNWLQQRPGQAPKL | | TYLNWLQQRPGQAPKL | |
| | | LMFQVSKPGIPDRFSGS | | LMFQVSKPGIPDRFSGSG | |
| | | GSETDFTLKISRVEAEDL | | SETDFTLKISRVEAEDLG | |
| | | GVYYCLQGTYFPHTFG | | VYYCLQGTYFPHTFGAG | |
| | | AGTKVEIK | | TKVEIKRTVAAPSVFIFP | |
| | | | | PSDEQLKSGTASVVCLL | |
| | | | | NNFYPREAKVQWKVDN | |
| | | | | ALQSGNSQESVTEQDSK | |
| | | | | DSTYSLSSTLTLSKADYE | |
| | | | | KHKVYACEVTHQGLSSP | |
| | | | | VTKSFNRGEC | |
| C2 | Grafted | DIVMTQTPLSLSVTPGQ | 11 | DIVMTQTPLSLSVTPGQP | 17 |
| | Humanized | PASISCKSSQSLLDSDGK | | ASISCKSSQSLLDSDGKT | |
| | LC | TFLYWYLQKPGQSPQLL | | FLYWYLQKPGQSPQLLI | |
| | | IYLVSSRFSPDRFSGSGS | | YLVSSRFSPDRFSGSGSG | |
| | | GTDFTLKISRVEAEDVG | | TDFTLKISRVEAEDVGV | |
| | | VYYCWQGTHFPHTFGQ | | YYCWQGTHFPHTFGQG | |
| | | GTKVEIK | | TKVEIKRTVAAPSVFIFP | |
| | | | | PSDEQLKSGTASVVCLL | |
| | | | | NNFYPREAKVQWKVDN | |
| | | | | ALQSGNSQESVTEQDSK | |
| | | | | DSTYSLSSTLTLSKADYE | |
| | | | | KHKVYACEVTHQGLSSP | |
| | | | | VTKSFNRGEC | |
| C2 | Humanized | DIVMTQTPLSLSVTPGQ | 12 | DIVMTQTPLSLSVTPGQP | 18 |
| | Grafted | PASISCKSSQSLLDSDGK | | ASISCKSSQSLLDSDGKT | |
| | Variant | TFLYWYLQKPGQSPQLL | | FLYWYLQKPGQSPQLLI | |
| | LC1 | IYLVSKLDSGVPDRFSG | | YLVSKLDSGVPDRFSGS | |
| | | SGSGTDFTLKISRVEAE | | GSGTDFTLKISRVEAED | |
| | | DVGVYYCWQGTHFPHT | | VGVYYCWQGTHFPHTF | |
| | | FGQGTKVEIK | | GQGTKVEIKRTVAAPSV | |
| | | | | FIFPPSDEQLKSGTASVV | |
| | | | | CLLNNFYPREAKVQWK | |
| | | | | VDNALQSGNSQESVTEQ | |
| | | | | DSKDSTYSLSSTLTLSKA | |
| | | | | DYEKHKVYACEVTHQG | |
| | | | | LSSPVTKSFNRGEC | |
| C2 | Humanized | DIVMTQTPLSLSVTPGQ | 13 | DIVMTQTPLSLSVTPGQP | 19 |
| | Grafted | PASISCKSSQSLLDSDGK | | ASISCKSSQSLLDSDGKT | |
| | Variant | TFLNWLLQKPGQSPQLL | | FLNWLLQKPGQSPQLLI | |
| | LC2 | IYLVSKLDSGVPDRFSG | | YLVSKLDSGVPDRFSGS | |
| | | SGSGTDFTLKISRVEAE | | GSGTDFTLKISRVEAED | |
| | | DVGVYYCWQGTHFPHT | | VGVYYCWQGTHFPHTF | |
| | | FGQGTKVEIK | | GQGTKVEIKRTVAAPSV | |
| | | | | FIFPPSDEQLKSGTASVV | |
| | | | | CLLNNFYPREAKVQWK | |
| | | | | VDNALQSGNSQESVTEQ | |
| | | | | DSKDSTYSLSSTLTLSKA | |
| | | | | DYEKHKVYACEVTHQG | |
| | | | | LSSPVTKSFNRGEC | |
| C2 | Humanized | DIVMTQTPLSLSVTPGQ | 14 | DIVMTQTPLSLSVTPGQP | 20 |
| | Grafted | PASISCKSSQSLLDSDGK | | ASISCKSSQSLLDSDGKT | |
| | Variant | TFLNWLLQKPGQSPQRL | | FLNWLLQKPGQSPQRLI | |
| | LC3 | IYLVSKLDSGVPDRFSG | | YLVSKLDSGVPDRFSGS | |
| | | SGSGTDFTLKISRVEAE | | GSGTDFTLKISRVEAED | |
| | | DVGVYYCWQGTHFPHT | | VGVYYCWQGTHFPHTF | |
| | | FGQGTKVEIK | | GQGTKVEIKRTVAAPSV | |
| | | | | FIFPPSDEQLKSGTASVV | |
| | | | | CLLNNFYPREAKVQWK | |
| | | | | VDNALQSGNSQESVTEQ | |
| | | | | DSKDSTYSLSSTLTLSKA | |
| | | | | DYEKHKVYACEVTHQG | |
| | | | | LSSPVTKSFNRGEC | |

**The Signal Sequence of SEQ ID Nos: 15-20 has been excluded from the mature full-length sequences shown. The signal sequence used to ensure expression of each of the mature full length light chains shown was METGLRWLLLVAVLKGVQC (SEQ ID NO: 38).

In some embodiments a CotH3 binding agent or the antigen binding portion of a CotH3 binding agent comprises a humanized light chain variable region having at least 70%, 75%, 85%, 86%, 87%, 88%, 89%, 90%, 91%, 92%, 93%, 94%, 95%, 96%, 97%, 98%, or at least 99% identity to a light chain variable region sequence of Table 2. In some embodiments a CotH3 binding agent or the antigen binding portion of a CotH3 binding agent comprises a humanized light chain having at least 70%, 75%, 85%, 86%, 87%, 88%, 89%, 90%, 91%, 92%, 93%, 94%, 95%, 96%, 97%, 98%, or at least 99% identity to a full-length light sequence of Table 2.

In certain embodiments, a CotH3 binding agent and/or an antigen binding portion of a CotH3 binding agent comprises one, two or three CDRs of a heavy chain variable region. In some embodiments a heavy chain variable region comprises one or more CDRs (e.g., one, two, three, or more CDRs). An amino acid sequences representing a CDR in a heavy chain variable region of an antibody or binding agent are referred to as a CDR-H1, CDR-H2, and CDR-H3, which are numbered sequentially (i.e., H1, H2 and H3) in the direction from the amino terminus (N-terminus) to the carboxy terminus (C-terminus) of a heavy chain variable region. For example, in a polypeptide representing a heavy chain variable region of a CotH3 binding agent, CDR-H1, when present, is often the most N-terminal CDR; CDR-H3, when present, is often the most C-terminal CDR; and CDR-H2, when present, is often located (i) between CDR-H1 and CDR-H3, (ii) on the N-terminal side of CDR-H3 or (iii) on the C-terminal side of CDR-H, of a heavy chain variable region. The terms “CDR-H1”, “CDR-H2” and “CDR-H3” refer to, in part, an amino acid sequence of a polypeptide identified as, or disclosed herein as, a complementarity determining region of a CotH3 binding agent (e.g., a CDR of a heavy chain variable region of a CotH3 binding agent). Non-limiting examples of amino acid sequences of a CDR-H1, CDR-H2 and CDR-H3 are provided in Table 3, respectively. A heavy chain variable region or antigen binding portion of a CotH3 binding agent described herein may comprise any combination of a CDR-H1, a CDR-H2, and a CDR-H3 disclosed herein, where the CotH3 binding agent retains specific binding to CotH3, or a portion thereof.

In certain embodiments, a binding agent comprises at least one heavy chain CDR-H1 comprising an amino acid sequence at least 70%, at least 80%, at least 90%, at least 95%, or 100% identical to a CDR-H1 selected from Table 3. In certain embodiments, a binding agent comprises a single heavy chain CDR-H1 comprising an amino acid sequence at least 70%, at least 80%, at least 90%, at least 95%, or 100% identical to a CDR-H1 selected from Table 3. In certain embodiments, a heavy chain variable region or antigen binding portion of a binding agent described herein comprises an amino acid sequence at least 70%, at least 80%, at least 90%, at least 95%, or 100% identical to a CDR-H1 selected from Table 3, and any other suitable CDR-H2 and/or CDR-H3 polypeptide sequence (e.g., a CDR-H2 or CDR-H3 selected from Table 3), where the binding agent retains specific binding to CotH3, or a portion thereof.

In certain embodiments, a binding agent comprises at least one heavy chain CDR-H2 comprising an amino acid sequence at least 70%, at least 80%, at least 90%, at least 95%, or 100% identical to a CDR-H2 selected from Table 3. In certain embodiments, a binding agent comprises a single heavy chain CDR-H2 comprising an amino acid sequence at least 70%, at least 80%, at least 90%, at least 95%, or 100% identical to a CDR-H2 selected from Table 3. In certain embodiments, a heavy chain variable region or antigen binding portion of a binding agent described herein comprises an amino acid sequence at least 70%, at least 80%, at least 90%, at least 95%, or 100% identical to a CDR-H2 selected from Table 3, and any other suitable CDR-H1 and/or CDR-H3 polypeptide sequence (e.g., a CDR-H1 or CDR-H3 selected from Table 3), where the binding agent retains specific binding to CotH3, or a portion thereof.

In some embodiments a binding agent comprises one or more heavy chain CDRs that are at least 70%, 75%, 85%, 86%, 87%, 88%, 89%, 90%, 91%, 92%, 93%, 94%, 95%, 96%, 97%, 98%, or at least 99% identical to any one of the CDR sequences listed in Table 3. In certain embodiments, a binding agent comprises at least one heavy chain CDR-H3 comprising an amino acid sequence at least 70%, at least 80%, at least 90%, at least 95%, or 100% identical to a CDR-H3 selected from Table 3. In certain embodiments, a binding agent comprises a single heavy chain CDR-H3 comprising an amino acid sequence at least 70%, at least 80%, at least 90%, at least 95%, or 100% identical to a CDR-H3 selected from Table 3. In certain embodiments, a heavy chain variable region or antigen binding portion of a binding agent described herein comprises an amino acid sequence at least 70%, at least 80%, at least 90%, at least 95%, or 100% identical to a CDR-H3 selected from Table 3, and any other suitable CDR-H1 and/or CDR-H2 polypeptide sequence (e.g., a CDR-H1 or CDR-H2 selected from Table 3), where the binding agent retains specific binding to CotH3, or a portion thereof.

In certain embodiments, a binding agent comprises two heavy chain CDRs independently selected from a CDR-H1, CDR-H2, and CDR-H3 of Table 3, where the binding agent specifically binding to CotH3, or a portion thereof. In certain embodiments, a binding agent comprises two heavy chain CDRs independently selected from a CDR-H1, CDR-H2, and CDR-H3 of any one clone listed in Table 3, where the binding agent specifically binding to CotH3, or a portion thereof. In certain embodiments, a binding agent comprises a CDR-H1, CDR-H2, and CDR-H3 comprising or consisting of an amino acid sequence at least 70%, at least 80%, at least 90%, at least 95%, or 100% identical to a CDR-H1, CDR-H2, and CDR-H3 selected from any one clone of Table 3, where the binding agent specifically binding to CotH3, or a portion thereof. In certain embodiments, a binding agent comprises a CDR-H1, CDR-H2, and CDR-H3 selected from any one clone of Table 3.

TABLE 3
Heavy Chain CDR Sequences

| | | | HC- | | HC- | | HC- |
|---|---|---|---|---|---|---|---|
| | | | CDR1 | | CDR2 | | CDR3 |
| | | | SEQ | | SEQ | | SEQ |
| Clone | | HC-CDR1 | ID | HC-CDR2 | ID | HC-CDR3 | ID |
| Number | Type | Sequence | NO: | Sequence | NO: | Sequence | NO: |
| C2 | Murine | GDSITSGY | 21 | IKYSGRT | 22 | ASRGY | 23 |
| C2 | Chimera | GDSITSGY | 21 | IKYSGRT | 22 | ASRGY | 23 |
| | Humanized | | | | | | |
| C2 | Grafted | GDSITSGY | 21 | IKYSGRT | 22 | ASRGY | 23 |
| | Chimera | | | | | | |
| | Humanized | | | | | | |

In some embodiments a CotH3 binding agent or the antigen binding portion of a CotH3 binding agent comprises a heavy chain variable region having an amino acid sequence at least 70%, 75%, 85%, 86%, 87%, 88%, 89%, 90%, 91%, 92%, 93%, 94%, 95%, 96%, 97%, 98%, or at least 99% identity to a heavy chain variable region sequence of Table 4. In some embodiments a CotH3 binding agent or the antigen binding portion of a CotH3 binding agent comprises a heavy chain variable region comprising or consisting of an amino acid sequence of a heavy chain variable region selected from Table 4. In some embodiments a CotH3 binding agent or the antigen binding portion of a CotH3 binding agent comprises a heavy chain sequence (Full Sequence) selected from Table 4.

TABLE 4
Variable Heavy Chain Sequences

| | | | | | HC Full |
|---|---|---|---|---|---|
| | | Heavy Chain | HCVR | Heavy Chain | Length |
| | | Variable Region | SEQ | (Full-length | SEQ ID |
| Clone | Type | (HCVR) | ID NO: | Sequence) | NO: |
| C2 | Murine | DLQLQESGPSL | 24 | | |
| | | VKPSQTLSLTC | | | |
| | | SVTGDSITSGY | | | |
| | | WNWIRKFPGN | | | |
| | | KLEHMGYIKY | | | |
| | | SGRTFYNPSLK | | | |
| | | SRVSITRDTSK | | | |
| | | NQYYLQLNSV | | | |
| | | TSEDTATYYC | | | |
| | | ASRGYWGQG | | | |
| | | TTLTVSS | | | |
| C2 | Humanized | DLQLQESGPSL | 24 | DLQLQESGPSLVKPSQTLSLTCSVT | 29 |
| | Chimera | VKPSQTLSLTC | | GDSITSGYWNWIRKFPGNKLEHM | |
| | (IgG1 | SVTGDSITSGY | | GYIKYSGRTFYNPSLKSRVSITRDT | |
| | Constant) | WNWIRKFPGN | | SKNQYYLQLNSVTSEDTATYYCAS | |
| | | KLEHMGYIKY | | RGYWGQGTTLTVSSASTKGPSVFP | |
| | | SGRTFYNPSLK | | LAPSSKSTSGGTAALGCLVKDYFP | |
| | | SRVSITRDTSK | | EPVTVSWNSGALTSGVHTFPAVLQ | |
| | | NQYYLQLNSV | | SSGLYSLSSVVTVPSSSLGTQTYIC | |
| | | TSEDTATYYC | | NVNHKPSNTKVDKKVEPKSCDKT | |
| | | ASRGYWGQG | | HTCPPCPAPELLGGPSVFLFPPKPK | |
| | | TTLTVSS | | DTLMISRTPEVTCVVVDVSHEDPE | |
| | | | | VKFNWYVDGVEVHNAKTKPREE | |
| | | | | QYNSTYRVVSVLTVLHQDWLNGK | |
| | | | | EYKCKVSNKALPAPIEKTISKAKG | |
| | | | | QPREPQVYTLPPSRDELTKNQVSL | |
| | | | | TCLVKGFYPSDIAVEWESNGQPEN | |
| | | | | NYKTTPPVLDSDGSFFLYSKLTVD | |
| | | | | KSRWQQGNVFSCSVMHEALHNH | |
| | | | | YTQKSLSLSPGK | |
| C2 | Humanized | QVQLQESGPG | 25 | QVQLQESGPGLVKPSETLSLTCTV | 30 |
| | Grafted HC | LVKPSETLSLT | | SGDSITSGYWSWIRQPPGKGLEWI | |
| | (IgG1 | CTVSGDSITSG | | GYIKYSGRTNYNPSLKSRVTISVDT | |
| | Constant) | YWSWIRQPPG | | SKNQFSLKLSSVTAADTAVYYCAS | |
| | | KGLEWIGYIK | | RGYWGQGTLVTVSSASTKGPSVFP | |
| | | YSGRTNYNPS | | LAPSSKSTSGGTAALGCLVKDYFP | |
| | | LKSRVTISVDT | | EPVTVSWNSGALTSGVHTFPAVLQ | |
| | | SKNQFSLKLSS | | SSGLYSLSSVVTVPSSSLGTQTYIC | |
| | | VTAADTAVYY | | NVNHKPSNTKVDKKVEPKSCDKT | |
| | | CASRGYWGQ | | HTCPPCPAPELLGGPSVFLFPPKPK | |
| | | GTLVTVSS | | DTLMISRTPEVTCVVVDVSHEDPE | |
| | | | | VKFNWYVDGVEVHNAKTKPREE | |
| | | | | QYNSTYRVVSVLTVLHQDWLNGK | |
| | | | | EYKCKVSNKALPAPIEKTISKAKG | |
| | | | | QPREPQVYTLPPSRDELTKNQVSL | |
| | | | | TCLVKGFYPSDIAVEWESNGQPEN | |
| | | | | NYKTTPPVLDSDGSFFLYSKLTVD | |
| | | | | KSRWQQGNVFSCSVMHEALHNH | |
| | | | | YTQKSLSLSPGK | |
| C2 | Humanized | QVQLQESGPG | 26 | QVQLQESGPGLVKPSETLSLTCTV | 31 |
| | Grafted | LVKPSETLSLT | | SGDSITSGYWSWIRQPPGKGLEWI | |
| | Variant HC1 | CTVSGDSITSG | | GYIKYSGRTNYNPSLKSRVTISRDT | |
| | | YWSWIRQPPG | | SKNQFSLKLSSVTAADTAVYYCAS | |
| | | KGLEWIGYIK | | RGYWGQGTLVTVSSASTKGPSVFP | |
| | | YSGRTNYNPS | | LAPSSKSTSGGTAALGCLVKDYFP | |
| | | LKSRVTISRDT | | EPVTVSWNSGALTSGVHTFPAVLQ | |
| | | SKNQFSLKLSS | | SSGLYSLSSVVTVPSSSLGTQTYIC | |
| | | VTAADTAVYY | | NVNHKPSNTKVDKKVEPKSCDKT | |
| | | CASRGYWGQ | | HTCPPCPAPELLGGPSVFLFPPKPK | |
| | | GTLVTVSSAST | | DTLMISRTPEVTCVVVDVSHEDPE | |
| | | KGPSVFPL | | VKFNWYVDGVEVHNAKTKPREE | |
| | | | | QYNSTYRVVSVLTVLHQDWLNGK | |
| | | | | EYKCKVSNKALPAPIEKTISKAKG | |
| | | | | QPREPQVYTLPPSRDELTKNQVSL | |
| | | | | TCLVKGFYPSDIAVEWESNGQPEN | |
| | | | | NYKTTPPVLDSDGSFFLYSKLTVD | |
| | | | | KSRWQQGNVFSCSVMHEALHNH | |
| | | | | YTQKSLSLSPGK | |
| C2 | Humanized | QVQLQESGPG | 27 | QVQLQESGPGLVKPSETLSLTCTV | 32 |
| | Grafted | LVKPSETLSLT | | SGDSITSGYWSWIRQPPGKGLEHIG | |
| | Variant HC2 | CTVSGDSITSG | | YIKYSGRTFYNPSLKSRVTISRDTS | |
| | | YWSWIRQPPG | | KNQFSLKLSSVTAADTAVYYCASR | |
| | | KGLEHIGYIKY | | GYWGQGTLVTVSSASTKGPSVFPL | |
| | | SGRTFYNPSLK | | APSSKSTSGGTAALGCLVKDYFPE | |
| | | SRVTISRDTSK | | PVTVSWNSGALTSGVHTFPAVLQS | |
| | | NQFSLKLSSVT | | SGLYSLSSVVTVPSSSLGTQTYICN | |
| | | AADTAVYYC | | VNHKPSNTKVDKKVEPKSCDKTH | |
| | | ASRGYWGQG | | TCPPCPAPELLGGPSVFLFPPKPKD | |
| | | TLVTVSSASTK | | TLMISRTPEVTCVVVDVSHEDPEV | |
| | | GPSVFPL | | KFNWYVDGVEVHNAKTKPREEQ | |
| | | | | YNSTYRVVSVLTVLHQDWLNGKE | |
| | | | | YKCKVSNKALPAPIEKTISKAKGQ | |
| | | | | PREPQVYTLPPSRDELTKNQVSLT | |
| | | | | CLVKGFYPSDIAVEWESNGQPENN | |
| | | | | YKTTPPVLDSDGSFFLYSKLTVDK | |
| | | | | SRWQQGNVFSCSVMHEALHNHYT | |
| | | | | QKSLSLSPGK | |
| C2 | Humanized | QVQLQESGPG | 28 | QVQLQESGPGLVKPSETLSLTCTV | 33 |
| | Grafted | LVKPSETLSLT | | SGDSITSGYWNWIRQPPGKGLEHI | |
| | Variant HC3 | CTVSGDSITSG | | GYIKYSGRTFYNPSLKSRVTISRDT | |
| | | YWNWIRQPPG | | SKNQFSLKLSSVTAADTAVYYCAS | |
| | | KGLEHIGYIKY | | RGYWGQGTLVTVSSASTKGPSVFP | |
| | | SGRTFYNPSLK | | LAPSSKSTSGGTAALGCLVKDYFP | |
| | | SRVTISRDTSK | | EPVTVSWNSGALTSGVHTFPAVLQ | |
| | | NQFSLKLSSVT | | SSGLYSLSSVVTVPSSSLGTQTYIC | |
| | | AADTAVYYC | | NVNHKPSNTKVDKKVEPKSCDKT | |
| | | ASRGYWGQG | | HTCPPCPAPELLGGPSVFLFPPKPK | |
| | | TLVTVSSASTK | | DTLMISRTPEVTCVVVDVSHEDPE | |
| | | GPSVFPL | | VKFNWYVDGVEVHNAKTKPREE | |
| | | | | QYNSTYRVVSVLTVLHQDWLNGK | |
| | | | | EYKCKVSNKALPAPIEKTISKAKG | |
| | | | | QPREPQVYTLPPSRDELTKNQVSL | |
| | | | | TCLVKGFYPSDIAVEWESNGQPEN | |
| | | | | NYKTTPPVLDSDGSFFLYSKLTVD | |
| | | | | KSRWQQGNVFSCSVMHEALHNH | |
| | | | | YTQKSLSLSPGK | |

**The Signal Sequence of SEQ ID Nos: 29-33 has been excluded from the mature full-length sequences shown. The signal sequence used to ensure expression of each of the mature full length heavy chains shown was MDTRAPTQLLGLLLLWLPGSRC (SEQ ID NO: 39).

In some embodiments a CotH3 binding agent or the antigen binding portion of a CotH3 binding agent comprises a humanized heavy chain variable region having at least 70%, 75%, 85%, 86%, 87%, 88%, 89%, 90%, 91%, 92%, 93%, 94%, 95%, 96%, 97%, 98%, or at least 99% identity to a heavy chain variable region sequence of Table 4, wherein the binding agent or binding portion thereof, binds specifically to CotH3. In some embodiments a CotH3 binding agent or the antigen binding portion of a CotH3 binding agent comprises a humanized heavy chain having at least 70%, 75%, 85%, 86%, 87%, 88%, 89%, 90%, 91%, 92%, 93%, 94%, 95%, 96%, 97%, 98%, or at least 99% identity to a full-length heavy sequence of Table 4, wherein the binding agent or binding portion thereof, binds specifically to CotH3.

In some embodiments a binding agent, or an antigen binding portion of a binding agent, comprises a CDR-L1, CDR-L2, and CDR-L3 comprising or consisting of an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of a CDR-L1, CDR-L2, and CDR-L3 selected from any one clone of Table 1; and a CDR-H1, CDR-H2, and CDR-H3 comprising or consisting of an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of a CDR-H1, CDR-H2, and CDR-H3 selected from any one corresponding clone of Table 3, wherein the binding agent or binding portion thereof, binds specifically to CotH3. In some embodiments a binding agent, or an antigen binding portion of a binding agent, comprises a CDR-L1, CDR-L2, and CDR-L3 selected from any one clone of Table 1 and a CDR-H1, CDR-H2, and CDR-H3 selected from any one corresponding clone of Table 3.

In some embodiments a binding agent, or an antigen binding portion of a binding agent, comprises a light chain variable region comprising of an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of a light chain variable region selected from any one clone or type of Table 2; and a heavy chain variable region comprising of an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of a heavy chain variable region selected from any one corresponding clone or type of Table 4, wherein the binding agent or binding portion thereof, binds specifically to a CotH3. In some embodiments a binding agent, or an antigen binding portion of a binding agent, comprises a light chain variable region selected from any one clone of Table 2 and a heavy chain variable region selected from any one corresponding clone of Table 4.

In some embodiments a binding agent, or an antigen binding portion of a binding agent, comprises a full length light chain comprising or consisting of an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of a full length light chain selected from any one clone of Table 2; and a full length heavy chain comprising or consisting of an amino acid sequence at least 70%, at least 75%, at least 85%, at least 86%, at least 87%, at least 88%, at least 89%, at least 90%, at least 91%, at least 92%, at least 93%, at least 94%, at least 95%, at least 96%, at least 97%, at least 98%, at least 99% or 100% identical to the amino acid sequence of a full length heavy chain selected from any one corresponding clone of Table 4, wherein the binding agent or binding portion thereof, binds specifically to CotH3. In some embodiments a binding agent, or an antigen binding portion of a binding agent, comprises a full-length light chain selected from any one clone of Table 2 and a full-length heavy chain selected from any one corresponding clone of Table 4.

The term “percent identical” or “percent identity” refers to sequence identity between two amino acid sequences. Identity can be determined by comparing a position in each sequence which may be aligned for purposes of comparison. When an equivalent position in the compared sequences is occupied by the same amino acid, then the molecules are identical at that position. When the equivalent site is occupied by the same or a similar amino acid residue (e.g., similar in steric and/or electronic nature), then the molecules can be referred to as homologous (similar) at that position. Expression as a percentage of homology, similarity, or identity refers to a function of the number of identical or similar amino acids at positions shared by the compared sequences. Expression as a percentage of homology, similarity, or identity refers to a function of the number of identical or similar amino acids at positions shared by the compared sequences. Various alignment algorithms and/or programs may be used, including FASTA, BLAST, or ENTREZ. FASTA and BLAST are available as a part of the GCG sequence analysis package (University of Wisconsin, Madison, Wis.), and can be used with, e.g., default settings. ENTREZ is available through the National Center for Biotechnology Information, National Library of Medicine, National Institutes of Health, Bethesda, Md. In one embodiment, the percent identity of two sequences can be determined by the GCG program with a gap weight of 1, e.g., each amino acid gap is weighted as if it were a single amino acid or nucleotide mismatch between the two sequences.

Other techniques for alignment are described in Methods in Enzymology, vol. 266: Computer Methods for Macromolecular Sequence Analysis (1996), ed. Doolittle, Academic Press, Inc., a division of Harcourt Brace & Co., San Diego, Calif., USA. In some embodiments an alignment program that permits gaps in the sequence is utilized to align the sequences. The Smith-Waterman is one type of algorithm that permits gaps in sequence alignments. See Meth. Mol. Biol. 70:173-187 (1997). Also, the GAP program using the Needleman and Wunsch alignment method can be utilized to align sequences. An alternative search strategy uses MPSRCH software, which runs on a MASPAR computer. MPSRCH uses a Smith-Waterman algorithm to score sequences on a massively parallel computer. This approach improves ability to pick up distantly related matches, and is especially tolerant of small gaps and nucleotide sequence errors. Nucleic acid-encoded amino acid sequences can be used to search both protein and DNA databases.

In some embodiments an antibody, a humanized antibody, a human/mouse chimeric antibody or a CDR grafted humanized antibody, or antigen binding portion thereof, comprises one or more light chain CDRs selected from Table 1 and one or more heavy chain CDRs selected from Table 3. In some embodiments an antibody, a humanized antibody, a human/mouse chimeric antibody or a CDR grafted humanized antibody, or antigen binding portion thereof, comprises one or more CDRs selected from a light chain variable region of Table 2 and one or more CDRs selected from a heavy chain variable region of Table 4. In some embodiments an antibody, a humanized antibody, a human/mouse chimeric antibody or a CDR grafted humanized antibody, or antigen binding portion thereof, comprises a CDR-L1, a CDR-L2, and a CDR-L3, each selected from any one light chain variable region of a clone or type listed in Table 2, and a CDR-H1, a CDR-H2, and a CDR-H3, each selected from any one of the heavy chain variable region of a corresponding clone or type listed in Table 4. An amino acid sequence of a CDR (e.g., a CDR-L1, CDR-L2, CDR-L3, CDR-H1, CDR-H2, and CDR-H3) can be identified within a heavy chain or light chain variable region disclosed herein by any suitable method described herein or known to those skilled in the art.

In some embodiments a binding agent comprises one or more suitable sequences selected from Tables 1-4 wherein the selected polypeptide sequence comprises 1 to 12, 1 to 10, 1 to 5, 5, 4, 3, 2 or 1 amino acid modifications, where an amino acid modification can be an amino acid addition, an amino acid deletion and/or an amino acid substitution. In some embodiments a binding agent comprises one or more suitable sequences selected from Tables 1-4 wherein the selected polypeptide sequence comprises 1 to 12, 1 to 10, 1 to 5, 5, 4, 3, 2 or 1 conservative amino acid substitutions, wherein the binding agent maintains specific binding to CotH3 or the peptide of SEQ ID NO:34. In some embodiments, a binding agent disclosed herein comprises one or more amino acid substitutions, wherein an amino acid is substituted with an amino acid analogue, non-native amino acid or amino acid derivative.

In certain embodiments, a binding agent, or antigen binding portion of a binding agent, comprises one or more framework regions (FR). In certain embodiments, a binding agent, or antigen binding portion of a binding agent, comprises one or more human framework regions (FR). Framework regions are often located between CDRs and/or flank CDR sequences of a heavy or light chain variable region of an antibody or binding agent. In mammals, a heavy chain variable region often comprises four framework regions and a light chain variable region often comprises four framework regions. Any suitable method can be used to identify one or more framework regions in an antibody, in a variable region of an antibody or in a binding agent. A binding agent may comprise synthetic or naturally occurring framework regions which are unmodified or modified (e.g., optimized) as discussed below.

In some embodiments a binding agent, or antigen binding portion thereof is chimeric, grafted and/or humanized. Chimeric, grafted and or humanized binding agents often comprise modified or substituted constant regions and/or framework regions while maintaining binding specificity to CotH3, or a portion thereof (e.g., SEQ ID NO:34). In some embodiments a binding agent, or antigen binding portion thereof, comprises constant regions, framework regions, or portions thereof, derived from a human antibody. In some embodiments a binding agent, or antigen binding portion thereof, comprises fully synthetic portions (e.g., one or more amino acids, or sequences of amino acids that are not found in native antibody molecules).

Naturally occurring framework regions, or portions thereof may be obtained from any suitable species. In certain embodiments the complementarity determining regions (CDRs) of the light and heavy chain variable regions of a binding agent, or an antigen binding portion thereof, is grafted into framework regions from the same, or another species. For example, one or more framework regions of a binding agent may be derived from a rodent species (e.g., a mouse or rat) or a primate species (e.g., a human).

In certain embodiments, the CDRs of the light and/or heavy chain variable regions of a binding agent, or an antigen binding portion thereof, can be grafted to consensus human framework regions. To create consensus human framework regions, in certain embodiments, framework regions from several human heavy chain or light chain amino acid sequences can be aligned to identify a consensus sequence. In certain embodiments, the heavy chain or light chain framework regions of an antibody or binding agent are replaced with one or more framework regions, or portions thereof, from a different heavy chain or light chain variable region. In some embodiments a binding agent, or antigen binding portion thereof, comprises one or more human framework regions. In certain embodiments a binding agent, or antigen binding portion thereof, comprises at least 1, 2, 3, 4, 5, 6, 7, 8, 9 or 10 human framework regions. In some embodiments a binding agent, or antigen binding portion thereof, comprises one or more mouse framework regions. In certain embodiments a binding agent, or antigen binding portion thereof, comprises at least 1, 2, 3, 4, 5, 6, 7, 8, 9 or 10 mouse framework regions. In certain embodiments a binding agent, or antigen binding portion thereof, comprises one or more human framework regions and one or more mouse framework regions.

Methods of generating chimeric, humanized and/or optimized antibodies or binding agents, for example by modifying, substituting or deleting framework regions, or portions thereof, are known. Non-limiting examples of CDR grafting are described, e.g., in U.S. Pat. Nos. 6,180,370, 6,054,297, 5,693,762, 5,859,205, 5,693,761, 5,565,332, 5,585,089, and 5,530,101, and in Jones et al, Nature, 321:522-525 (1986); Verhoeyen et al, Science, 239:1534-1536 (1988), and Winter, FEBS Letts., 430:92-94 (1998). Additional non-limiting examples of generating chimeric, grafted and/or humanized binding agents include U.S. Pat. Nos. 5,530,101; 5,707,622; 5,994,524; 6,245,894; Queen et al., (1988) PNAS 86:10029-10033; Riechmann et al., Nature (1988) 332:323-327; Antibody Engineering: Methods and Protocols, Vol. 248 of Methods in molecular biology, edited by Benny K. C. Lo, Springer Science & Business Media, (2004); and Antibody Engineering, Vol. 1, Roland E. Kontermann, Stefan Dubel, Edition 2, Publisher Springer Science & Business Media, (2010). In some embodiments a binding agent can be humanized by exchanging one or more framework regions, or portions thereof (e.g., one or more amino acids), with one or more framework regions, or portions thereof from a human antibody. In certain embodiments, an antibody or binding agent can be humanized or grafted by transferring one or more CDRs (e.g., 1, 2, 3, 4, 5 or all 6 CDRs) from a donor binding agent (e.g., a mouse monoclonal antibody) to an acceptor binding agent (e.g., a human antibody) while retaining the binding specificity of the donor binding agent. In certain embodiments, the process of making a chimeric, grafted or humanized binding agent comprises making one or more amino acid substitutions, additions or deletions in a constant region or framework region of a binding agent. In certain embodiments, techniques such as “reshaping”, “hyperchimerization,” or “veneering/resurfacing” can be used to produce humanized binding agents. (e.g., see Vaswami et al, Annals of Allergy, Asthma, & Immunol. 81:105 (1998); Roguska et al, Prot. Engin., 9:895-904 (1996); and U.S. Pat. No. 6,072,035). In some aspects, a binding agent is modified by a method discussed above, or by another suitable method, to reduce immunogenicity (e.g., see Gilliland et al, J. Immunol, 62(6):3663-71 (1999)).

In certain embodiments, an amino acid sequence of a binding agent is modified to optimize binding affinity for a target (e.g., CotH3), species cross-reactivity, solubility and/or function (e.g., agonist activity, or lack thereof). In some embodiments a specific combination of CDRs disclosed herein can be optimized for binding to a CotH3, CotH2, and/or to optimize a function or characteristic of the binding agent. For example, a light chain variable region disclosed herein (e.g., a light chain variable region of Table 2) can be co-expressed, using a suitable expression system, with a different heavy chain variable region selected from Table 4 and the resulting binding agents can be tested for binding to CotH3, CotH2, cross-reactivity to a CotH derived from difference species, solubility and/or function such that an optimized binding agent with a desired characteristic can be selected. In yet another example, a light chain variable region disclosed herein (e.g., a light chain variable region of Table 2) can be co-expressed, using a suitable expression system, with a library of heavy chain variable regions comprising a CDR-H1 and CDR-H2 of a heavy chain variable region selected from Table 4, and a CDR-H3 from Table 2 comprising one or more amino acid substitutions, deletions or insertions. The resulting binding agents can be screened for binding to CotH3 and/or for a desired affinity, specificity or function. Once an optimized binding agent having the desired properties is identified, the amino acid sequence of the binding agent can readily be identified using a suitable method. The above described screening methods, and other similar methods can be used to identify binding agents comprising specific combinations of CDRs, or specific optimized CDR sequences (e.g., CDR sequences comprising amino acid substitutions, additions or deletions) that provide a binding agent with improved binding specificity, binding affinity and/or function. Exemplary methods of screening and optimizing binding agents are described in, for example, Portolano et al., (1993) Journal of Immunology 150:880-887, and Clarkson et al., (1991) Nature 352:624-628, which methods can be used to identify a CotH3 binding agent having a sequence similar to one described in Tables 1-4, wherein the CotH3 binding agent maintains specific binding to CotH3.

In certain embodiments, a binding agent is modified to eliminate or add glycosylation sites in order to optimize affinity and/or function of a binding agent (e.g., see Co et al, Mol. Immunol, 30:1361-1367 (1993)). In some embodiments the number and/or type of glycosylation sites in a binding agent is modified or altered. An N-linked glycosylation site is often characterized by the sequence Asn-X-Ser or Asn-X-Thr, where the amino acid residue designated as X can be any amino acid residue except proline. The substitution of amino acid residues to create this sequence provides a potential new site for the addition of an N-linked carbohydrate chain. Alternatively, substitutions which eliminate this sequence will remove an existing N-linked carbohydrate chain. Also provided in certain embodiments is a rearrangement of N-linked carbohydrate chains where one or more N-linked glycosylation sites (typically those that are naturally occurring) are eliminated and one or more new N-linked sites are created. In some embodiments a binding agent is modified by deleting one or more cysteine residues or substituting one or more cysteine residues for another amino acid (e.g., serine) as compared to an unmodified binding agent. In certain embodiments cysteine variants can be useful for optimizing expression, secretion, and/or solubility.

In certain embodiments a binding agent is modified to include certain amino acid additions, substitutions, or deletions designed or intended, for example, to reduce susceptibility of a binding agent to proteolysis, reduce susceptibility of a binding agent to oxidation, increase serum half-life and/or confer or modify other physicochemical, pharmacokinetic or functional properties of a binding agent.

In some embodiments a binding agent specifically binds to a Mucorales CotH3, or portion thereof. In certain embodiments, a binding agent described herein specifically binds to a Mucorales CotH3, or portion thereof, with a binding affinity (KD) of 10⁻⁵ M or less, 10⁻⁶ M or less, 10⁻⁷ M or less, 10⁻⁸ M or less, 50 nM or less, 10 nM or less, 5 nM or less, or 1 nM or less. In certain embodiments, a binding agent described herein specifically binds to a Mucorales CotH3, or portion thereof, with a binding affinity (KD) from about 10⁻⁵ to 10⁻¹⁵ M, 10⁻⁶ to 10⁻⁵ M, 10⁻⁷ to 10⁻¹⁵ M, 10⁻⁹ to 10⁻¹⁵ M, 10⁻⁹ to 10⁻¹⁴ M, 10⁻⁹ to 10⁻¹³ M, or 10⁻⁹ to about 10⁻¹² M. In certain aspects a binding agent specifically binds to a naturally occurring CotH3 or naturally occurring CotH3 variant. In certain aspects, a binding agent specifically binds to a CotH3 comprising one or more amino acid substitutions, additions or deletions. In certain embodiments a binding agent specifically binds to CotH3 of R. oryzae. In certain embodiments a binding agent specifically binds to a mature/processed CotH3 polypeptide. In certain embodiments, a binding agent described herein specifically binds to one or more polypeptides having at least 70%, at least 80%, at least 90% or at least 95% identity to an amino acid sequence of SEQ ID NO:34, 35, 36, and/or 37 with a binding affinity (KD) of 50 nM or less, 10 nM or less, 5 nM or less, or 1 nM or less. In certain embodiments, a binding agent described herein specifically binds to one or more polypeptides of SEQ ID NO:34, 35, 36, and/or 37, or one or more subsequences thereof with a binding affinity (KD) of 50 nM or less, 10 nM or less, 5 nM or less, or 1 nM or less.

The term “specifically binds” refers to a binding agent that binds to a target peptide in preference to binding other molecules or other peptides as determined by, for example, as determined by a suitable in vitro assay (e.g., an Elisa, Immunoblot, Flow cytometry, and the like). A specific binding interaction discriminates over non-specific binding interactions by about 2-fold or more, often about 10-fold or more, and sometimes about 100-fold or more, 1000-fold or more, 10,000-fold or more, 100,000-fold or more, or 1,000,000-fold or more.

In some embodiments a binding agent that specifically binds to CotH3, or a portion thereof, is a binding agent that binds CotH3, or a portion thereof (e.g., SEQ ID NO:34), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM. In some embodiments a binding agent that specifically binds to CotH3, or a portion thereof, is a binding agent that binds human CotH3, or a portion thereof (e.g., SEQ ID NO:34), with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM.

In some embodiments a binding agent that specifically binds to CotH3, or a portion thereof, is a binding agent that binds specifically to CotH2, or a portion thereof, with a binding affinity constant (KD) equal to or less than 100 nM, equal to or less than 50 nM, equal to or less than 25 nM, equal to or less than 10 nM, equal to or less than 5 nM, equal to or less than 1 nM, equal to or less than 900 pM, equal to or less than 800 pM, equal to or less than 750 pM, equal to or less than 700 pM, equal to or less than 600 pM, equal to or less than 500 pM, equal to or less than 400 pM, equal to or less than 300 pM, equal to or less than 200 pM, or equal to or less than 100 pM.

In some embodiments a binding agent comprises a label. As used herein, the terms “label” or “labeled” refers to incorporation of a detectable marker, e.g., by incorporation of a labeled amino acid or attachment to a polypeptide of biotin moieties that can be detected by labeled avidin (e.g., streptavidin containing a fluorescent marker or enzymatic activity that can be detected by optical or colorimetric methods). In certain embodiments, a label or marker can be attached to a binding agent to generate a diagnostic agent. A binding agent can be attached covalently or non-covalently to any suitable label or marker. Various methods of labeling polypeptides and glycoproteins are known to those skilled in the art and can be used. Non-limiting examples of labels for polypeptides include, but are not limited to fluorescent labels, enzymatic labels (e.g., horseradish peroxidase, 0-galactosidase, luciferase, alkaline phosphatase), chemiluminescent labels, a metallic label, a chromophore, an electro-chemiluminescent label, a phosphorescent label, a quencher (e.g., a fluorophore quencher), a fluorescence resonance energy transfer (FRET) pair (e.g., donor and acceptor), a dye, an enzyme substrate, a small molecule, a mass tag, quantum dots, nanoparticles, biotinyl groups, predetermined polypeptide epitopes recognized by a secondary reporter (e.g., leucine zipper pair sequences, binding sites for secondary antibodies, metal binding domains, epitope tags), the like or combinations thereof.

In some embodiments a binding agent comprises a suitable carrier. A binding agent can be attached covalently or non-covalently to a suitable carrier. Non-limiting examples of a carrier include agents or molecules that alter or extend the in vivo half-life of a binding agent, polyethylene glycol, glycogen (e.g., by glycosylation of a binding agent), a dextran, a carrier or vehicle described in U.S. Pat. No. 6,660,843, the like or combinations thereof.

In some embodiments a label or carrier is bound to a binding agent by use of a suitable linker. Non-limiting examples of a suitable linker include silanes, thiols, phosphonic acid, polyethylene glycol (PEG), amino acids and peptides, polymers thereof, derivatives thereof, the like and combinations thereof. Methods of attaching two or more molecules using a linker are to those skilled in the art and are sometimes referred to as “crosslinking.”

In some embodiments a binding agent, or binding portion thereof is attached to a substrate (e.g., a polymer, a non-organic material, silicon, a bead, a surface, a nanoparticle, and the like).

In some embodiments a label, carrier or linker is attached to a suitable thiol group of a binding agent (e.g., a thiol group of a cysteine residue). Any suitable amino acid residue of a constant region or framework region of a binding agent can be substituted with an amino acid residue containing a thiol group (e.g., a cysteine) for the purpose of attaching a label, carrier or linker.

### Exemplary Methods

In some embodiments, provided herein are methods of preventing or treating a Mucorales infection in a subject comprising administering to the subject a therapeutically effective amount of a composition, a pharmaceutical composition or a binding agent described herein to the subject. In some embodiments a composition, pharmaceutical composition or binding agent described herein is used to prevent or treat a subject (e.g., a subject in need) who has, is suspected of having, or is at risk of having a Mucorales infection. In some embodiments, a Mucorales infection is caused by they presence of a Mucorales species selected from A. idahoensis, A. corymbifera, Apophysomyces elegans, Actinomucor elegans, A. rouxii, B. circina, B. multispora, C. brefeldii, C. angarensis, Cunninghamella bertholletiae (C. bertholletiae), Choanephora cucurbitarum, C. recurvatus, D. fulva, E. anomalus, H. elegans, H. assamensis, K. cordensis, Lichtheimia corymbifera (L. corymbifera), Lichtheimia ramosa, M. ambiguus, Mucor amphibiorum, Mucor circinelloides, M. verticillata, Parasitella parasitica, P. agaricine, P. anomala, P. circinans, Phycomyces blakesleeanus, S. umbellata, S. megalocarpus, T. elegans, T. indicae-seudaticae, Z. californiensis, Rhizomucor endophyticus, Rhizopus javensis, R. azygosporus, Rhizopus caespitosus, Rhizopus homothallicus, Rhizopus oryzae, Rhizopus stolonifer, Rhizopus reflexus, Rhizopus microsporus (e.g., var. rhizopodiformis), and Rhizopus schipperae. In some embodiments, a Mucorales infection that can be prevented or treated by a method herein is a Mucorales infection is caused by the presence of a Mucorales species of the genus Rhizopus. In some embodiments, a Mucorales infection that can be prevented or treated by a method herein is a Mucorales infection is caused by the presence of a Mucorales species selected from Rhizopus oryzae, Lichtheimia corymbifera, Cunninghamella bertholetiae, and R. mirosporus. In certain embodiments, a Mucorales species is a species that expresses a CotH3 polypeptide. In certain embodiments, a Mucorales species is a species that expresses a CotH2 polypeptide. In certain embodiments, a Mucorales species is a species that expresses a CotH protein comprising the amino acid sequence set forth in SEQ ID NO:34.

A Mucorales species that can be prevented or treated by a method described herein can be quickly assayed for expression of CotH3, CotH2, or a polypeptide comprising the amino acid sequence of SEQ ID NO:34 using a suitable detection method (e.g., whole cell ELISA, FACS, any suitable immunoassay, and the like). In certain embodiments, a detection method utilizes a binding agent described herein.

In some embodiments a method of treating a Mucorales infection comprises administration of a binding agent described herein and an anti-fungal agent. In certain embodiments a composition comprises one or more binding agents described herein and one or more anti-fungal agents.

Non-limiting examples of anti-fungal agents include amphotericin B, candicidin, filipin, hamycin, natamycin, nystatin, rimocidin, imidazoles (e.g., bifonazole, butoconazole, clotrimazole, econazole, fenticonazoleisoconazole, ketoconazole, luliconazole, miconazole, omoconazole, oxiconazole, sertaconazole, sulconazole, tioconazole, and the like), triazoles (e.g., albaconazole, efinaconazole, epoxiconazole, fluconazole, isavuconazole, itraconazole, posaconazole, propiconazole, ravuconazole, terconazole, voriconazole, and the like), thiazoles, (e.g., abafungin), allylamines (e.g., amorolfin, butenafine, naftifine, and terbinafine), echinocandins (e.g., anidulafungin, caspofungin, micafungin), benzoic acid (e.g., combined with a keratolytic agent such as in whitfield's ointment), ciclopirox (ciclopirox olamine), flucytosine, 5-fluorocytosine, griseofulvin, haloprogin, tolnaftate, undecylenic acid, crystal violet, Balsam of Peru, triterpenoids, the like or combinations thereof. Amphotericin B can be deoxy cholate formulation or a lipid formulations. In some embodiments Amphotericin B comprises liposomal Amphotericin B. In certain embodiments Amphotericin B comprises a lipid complex of Amphotericin B.

### Compositions

In some embodiments, presented herein is a composition or pharmaceutical composition comprising one or more binding agents described herein.

In some embodiments, the disclosure provides a composition or pharmaceutical composition comprising one or more binding agents and an anti-fungal agent described herein.

In one embodiment, the disclosure provides a composition or pharmaceutical composition comprising one or more binding agents and posaconazole.

In one embodiment, the disclosure provides a composition or pharmaceutical composition comprising one or more binding agents and LAmB (liposomal amphotericin B).

A pharmaceutical composition can be formulated for a suitable route of administration. In some embodiments a pharmaceutical composition is formulated for subcutaneous (s.c.), intradermal, intramuscular, intraperitoneal and/or intravenous (i.v.) administration. In certain embodiments, a pharmaceutical composition can contain formulation materials for modifying, maintaining, or preserving, for example, the pH, osmolarity, viscosity, clarity, color, isotonicity, odor, sterility, stability, rate of dissolution or release, adsorption or penetration of the composition. In certain embodiments, suitable formulation materials include, but are not limited to, amino acids (such as glycine, glutamine, asparagine, arginine or lysine); antimicrobials; antioxidants (such as ascorbic acid, sodium sulfite or sodium hydrogen-sulfite); buffers (such as borate, bicarbonate, Tris-HCl, citrates, phosphates (e.g., phosphate buffered saline) or suitable organic acids); bulking agents (such as mannitol or glycine); chelating agents (such as ethylenediamine tetraacetic acid (EDTA)); complexing agents (such as caffeine, polyvinylpyrrolidone, beta-cyclodextrin or hydroxypropyl-beta-cyclodextrin); proteins (such as serum albumin, gelatin or immunoglobulins); coloring, flavoring and diluting agents; emulsifying agents; hydrophilic polymers (such as polyvinylpyrrolidone); low molecular weight polypeptides; salt-forming counter ions (such as sodium); solvents (such as glycerin, propylene glycol or polyethylene glycol); diluents; excipients and/or pharmaceutical adjuvants (Remington's Pharmaceutical Sciences, 18th Ed., A.R. Gennaro, ed., Mack Publishing Company (1995)).

In certain embodiments, a pharmaceutical composition comprises a suitable excipient, non-limiting example of which include anti-adherents (e.g., magnesium stearate), a binder, fillers, monosaccharides, disaccharides, other carbohydrates (e.g., glucose, mannose or dextrins), sugar alcohols (e.g., mannitol or sorbitol), coatings (e.g., cellulose, hydroxypropyl methylcellulose (HPMC), microcrystalline cellulose, synthetic polymers, shellac, gelatin, corn protein zein, enterics or other polysaccharides), starch (e.g., potato, maize or wheat starch), silica, colors, disintegrants, flavors, lubricants, preservatives, sorbents, sweeteners, vehicles, suspending agents, surfactants and/or wetting agents (such as pluronics, PEG, sorbitan esters, polysorbates such as polysorbate 20, polysorbate 80, triton, tromethamine, lecithin, cholesterol, tyloxapal), stability enhancing agents (such as sucrose or sorbitol), and tonicity enhancing agents (such as alkali metal halides, sodium or potassium chloride, mannitol, sorbitol), and/or any excipient disclosed in Remington's Pharmaceutical Sciences, 18th Ed., A.R. Gennaro, ed., Mack Publishing Company (1995). The term “binder” as used herein refers to a compound or ingredient that helps keeps a pharmaceutical mixture combined. Suitable binders for making pharmaceutical formulations and are often used in the preparation of pharmaceutical tablets, capsules and granules are known to those skilled in the art. For clarification, the term “binding agent” as used herein does not refer to a “binder” that is used in certain pharmaceutical formulations. Although a pharmaceutical composition, in certain embodiments, may comprise a binding agent that specifically binds CotH3 as well as a binder.

In some embodiments a pharmaceutical composition comprises a suitable pharmaceutically acceptable additive and/or carrier. Non-limiting examples of suitable additives include a suitable pH adjuster, a soothing agent, a buffer, a sulfur-containing reducing agent, an antioxidant and the like. Non-limiting examples of a sulfur-containing reducing agent includes those having a sulfhydryl group such as N-acetylcysteine, N-acetylhomocysteine, thioctic acid, thiodiglycol, thioethanolamine, thioglycerol, thiosorbitol, thioglycolic acid and a salt thereof, sodium thiosulfate, glutathione, and a C1-C7 thioalkanoic acid. Non-limiting examples of an antioxidant include erythorbic acid, dibutylhydroxytoluene, butylhydroxyanisole, alpha-tocopherol, tocopherol acetate, L-ascorbic acid and a salt thereof, L-ascorbyl palmitate, L-ascorbyl stearate, sodium bisulfite, sodium sulfite, triamyl gallate and propyl gallate, as well as chelating agents such as disodium ethylenediaminetetraacetate (EDTA), sodium pyrophosphate and sodium metaphosphate. Furthermore, diluents, additives and excipients may comprise other commonly used ingredients, for example, inorganic salts such as sodium chloride, potassium chloride, calcium chloride, sodium phosphate, potassium phosphate and sodium bicarbonate, as well as organic salts such as sodium citrate, potassium citrate and sodium acetate.

The pharmaceutical compositions used herein can be stable over an extended period of time, for example on the order of months or years. In some embodiments a pharmaceutical composition comprises one or more suitable preservatives. Non-limiting examples of preservatives include benzalkonium chloride, benzoic acid, salicylic acid, thimerosal, phenethyl alcohol, methylparaben, propylparaben, chlorhexidine, sorbic acid, hydrogen peroxide, the like and/or combinations thereof. A preservative can comprise a quaternary ammonium compound, such as benzalkonium chloride, benzoxonium chloride, benzethonium chloride, cetrimide, sepazonium chloride, cetylpyridinium chloride, or domiphen bromide (BRADOSOL®). A preservative can comprise an alkyl-mercury salt of thiosalicylic acid, such as thimerosal, phenylmercuric nitrate, phenylmercuric acetate or phenylmercuric borate. A preservative can comprise a paraben, such as methylparaben or propylparaben. A preservative can comprise an alcohol, such as chlorobutanol, benzyl alcohol or phenyl ethyl alcohol. A preservative can comprise a biguanide derivative, such as chlorohexidine or polyhexamethylene biguanide. A preservative can comprise sodium perborate, imidazolidinyl urea, and/or sorbic acid. A preservative can comprise stabilized oxychloro complexes, such as known and commercially available under the trade name PURITE®. A preservative can comprise polyglycol-polyamine condensation resins, such as known and commercially available under the trade name POLYQUART® from Henkel KGaA. A preservative can comprise stabilized hydrogen peroxide. A preservative can be benzalkonium chloride. In some embodiments a pharmaceutical composition is free of preservatives.

In some embodiments a composition, pharmaceutical composition or binding agent is substantially free of blood, or a blood product contaminant (e.g., blood cells, platelets, polypeptides, minerals, blood borne compounds or chemicals, and the like). In some embodiments a composition, pharmaceutical composition or binding agent is substantially free of serum and serum contaminants (e.g., serum proteins, serum lipids, serum carbohydrates, serum antigens and the like). In some embodiments a composition, pharmaceutical composition or binding agent is substantially free a pathogen (e.g., a virus, parasite or bacteria). In some embodiments a composition, pharmaceutical composition or binding agent is substantially free of endotoxin. In some embodiments a composition, pharmaceutical composition or binding agent is sterile. In certain embodiments, a composition or pharmaceutical composition comprises a binding agent that specifically binds a domain of CotH3 and a diluent (e.g., phosphate buffered saline). In certain embodiments, a composition or pharmaceutical composition comprises a binding agent that specifically binds a domain of CotH3 and an excipient, (e.g., sodium citrate dehydrate, or polyoxyethylene-sorbitan-20 mono-oleate (polysorbate 80)).

The pharmaceutical compositions described herein may be configured for administration to a subject in any suitable form and/or amount according to the therapy in which they are employed. For example, a pharmaceutical composition configured for parenteral administration (e.g., by injection or infusion), may take the form of a suspension, solution or emulsion in an oily or aqueous vehicle and it may contain formulation agents, excipients, additives and/or diluents such as aqueous or non-aqueous solvents, co-solvents, suspending solutions, preservatives, stabilizing agents and or dispersing agents. In some embodiments a pharmaceutical composition suitable for parental administration may contain one or more excipients. In some embodiments a pharmaceutical composition is lyophilized to a dry powder form. In some embodiments a pharmaceutical composition is lyophilized to a dry powder form, which is suitable for reconstitution with a suitable pharmaceutical solvent (e.g., water, saline, an isotonic buffer solution (e.g., PBS), and the like). In certain embodiments, reconstituted forms of a lyophilized pharmaceutical composition are suitable for parental administration (e.g., intravenous administration) to a mammal.

In certain embodiments, a pharmaceutical composition is configured for oral administration and may be formulated as a tablet, microtablet, minitablets, micropellets, powders granules, capsules (e.g., capsules filled with microtablets, micropellets, powders or granules), emulsions or solutions. Pharmaceutical compositions configured for oral administration may comprise suitable coatings to delay or sustain release of the active ingredient (e.g., a binding agent), non-limiting examples of which include enteric coatings such as fatty acids, waxes, shellac, plastics, methyl acrylate-methacrylic acid copolymers, cellulose acetate phthalate (CAP), cellulose acetate succinate, hydroxypropyl methyl cellulose phthalate, hydroxypropyl methyl cellulose acetate succinate (hypromellose acetate succinate), polyvinyl acetate phthalate (PVAP), methyl methacrylate-methacrylic acid copolymers, cellulose acetate trimellitate, sodium alginate, zein, plant fibers, the like and combinations thereof.

In some embodiments a pharmaceutical compositions described herein may be configured for topical administration and may include one or more of a binding and/or lubricating agent, polymeric glycols, gelatins, cocoa-butter or other suitable waxes or fats. In some embodiments a pharmaceutical composition described herein is incorporated into a topical formulation containing a topical carrier that is generally suited to topical drug administration and comprising any suitable material known to those skilled in the art. In certain embodiments, a topical formulation of a pharmaceutical composition is formulated for administration of a binding agent from a topical patch.

In certain embodiments, an optimal pharmaceutical composition will be determined by one skilled in the art depending upon, for example, the intended route of administration, delivery format and desired dosage (see e.g., Remington's Pharmaceutical Sciences, supra). In certain embodiments, such compositions may influence the physical state, stability, rate of in vivo release and rate of in vivo clearance of the antibodies of the invention.

Any suitable method of administering a composition, pharmaceutical composition or binding agent to a subject can be used. The exact formulation and route of administration for a composition for use according to the methods of the invention described herein can be chosen by the individual physician in view of the subject's condition. See, e.g., Fingl et al. 1975, in “The Pharmacological Basis of Therapeutics,” Ch. 1, p. 1; which is incorporated herein by reference in its entirety. Any suitable route of administration can be used for administration of a pharmaceutical composition or a binding agent described herein. Non-limiting examples of routes of administration include topical or local (e.g., transdermally or cutaneously, (e.g., on the skin or epidermis), in or on the eye, intranasally, transmucosally, in the ear, inside the ear (e.g., behind the ear drum)), enteral (e.g., delivered through the gastrointestinal tract, e.g., orally (e.g., as a tablet, capsule, granule, liquid, emulsification, lozenge, or combination thereof), sublingual, by gastric feeding tube, rectally, and the like), by parenteral administration (e.g., parenterally, e.g., intravenously, intra-arterially, intramuscularly, intraperitoneally, intradermally, subcutaneously, intracavity, intracranial, intra-articular, into a joint space, intracardiac (into the heart), intracavernous injection, intralesional (into a skin lesion), intraosseous infusion (into the bone marrow), intrathecal (into the spinal canal), intrauterine, intravaginal, intravesical infusion, intravitreal), the like or combinations thereof.

In some embodiments a composition herein is provided to a subject. A composition that is provided to a subject is sometimes provided to a subject for self-administration or for administration to a subject by another (e.g., a non-medical professional). For example a composition described herein can be provided as an instruction written by a medical practitioner that authorizes a subject to be provided a composition or treatment described herein (e.g., a prescription). In another example, a composition can be provided to a subject where the subject self-administers a composition orally, intravenously or by way of an inhaler, for example.

Alternately, one can administer compositions for use according to the methods of the invention in a local rather than systemic manner, for example, via direct application to the skin, mucous membrane or region of interest for treating, including using a depot or sustained release formulation.

In some embodiments a pharmaceutical composition comprising a binding agent can be administered alone (e.g., as a single active ingredient (AI or e.g., as a single active pharmaceutical ingredient (API)). In other embodiments, a pharmaceutical composition comprising a binding agent can be administered in combination with one or more additional AIs/APIs, for example, as two separate compositions or as a single composition where the one or more additional AIs/APIs are mixed or formulated together with the binding agent in a pharmaceutical composition.

A pharmaceutical composition can be manufactured by any suitable manner, including, e.g., by means of conventional mixing, dissolving, granulating, dragee-making, levigating, emulsifying, encapsulating, entrapping or tableting processes.

In some embodiments a pharmaceutical composition comprising a binding agent is administered at a suitable frequency or interval as needed to obtain an effective therapeutic outcome. An effective therapeutic outcome can be determined by monitoring a Mucorales infection. Accordingly, in certain embodiments, a decrease in the number, viability, size, growth, or mitosis of fungal cells in a subject is considered an effective therapeutic outcome. In some embodiments, a pharmaceutical composition comprising a binding agent can be administered hourly, once a day, twice a day, three times a day, four times a day, five times a day, and/or at regular intervals, for example, every day, every other day, three times a week, weekly, every other week, once a month and/or simply at a frequency or interval as needed or recommended by a medical professional.

In some embodiments, an amount of a binding agent in a composition is an amount needed to obtain an effective therapeutic outcome. In certain embodiments, the amount of a binding agent in a composition (e.g., a pharmaceutical composition) is an amount sufficient to prevent, treat, reduce the severity of, delay the onset of, and/or alleviate a symptom of a Mucorales infection, as contemplated herein.

A “therapeutically effective amount” means an amount sufficient to obtain an effective therapeutic outcome and/or an amount necessary sufficient to prevent, treat, reduce the severity of, delay the onset of, and/or alleviate a symptom of a Mucorales infection. In certain embodiments, a “therapeutically effective amount” means an amount sufficient to terminate the growth of, and/or slow the growth of a Mucorales species in or on a subject. In certain embodiments, a “therapeutically effective amount” means an amount sufficient to inhibit the replication of, and/or induce the death of one or more fungal cells in or on a subject. Determination of a therapeutically effective amount is well within the capability of those skilled in the art, especially in light of the detailed disclosure provided herein.

In some embodiments, an amount of a binding agent in a composition is an amount that is at least a therapeutically effective amount and an amount low enough to minimize unwanted adverse reactions. The exact amount of a binding agent or combinations of active agents required will vary from subject to subject, depending on age, weight, and general condition of a subject, the severity of the condition being treated, and the particular combination of drugs administered. Thus, it is not always possible to specify an exact therapeutically effective amount to treat a Mucorales infection in a diverse group of subjects. As is well known, the specific dosage for a given subject under specific conditions and for a specific infection will routinely vary, but determination of the optimum amount in each case can readily be accomplished by simple routine procedures. Thus, a therapeutically effective amount of a binding agent used to treat a Mucorales infection may be determined by one of ordinary skill in the art using routine experimentation.

In certain embodiments, an amount of a binding agent in a composition is administered at a suitable therapeutically effective amount or a dose (e.g., at a suitable volume and concentration, which sometimes depends, in part, on a particular route of administration). Within certain embodiments, a binding agent (e.g., a binding agent in a composition) can be administered at a dose from about 0.01 mg/kg (e.g., per kg body weight of a subject) to 500 mg/kg, 0.1 mg/kg to 500 mg/kg, 0.1 mg/kg to 400 mg/kg, 0.01 mg/kg to 300 mg/kg, 0.1 mg/kg to 300 mg/kg, 0.1 mg/kg to 200 mg/kg, 0.1 mg/kg to 150 mg/kg, 0.1 mg/kg to 100 mg/kg, 0.1 mg/kg to 75 mg/kg, 0.1 mg/kg to 50 mg/kg, 0.1 mg/kg to 25 mg/kg, 0.1 mg/kg to 10 mg/kg, 0.1 mg/kg to 5 mg/kg or 0.1 mg/kg to 1 mg/kg. In some aspects the amount of a binding agent can be about 10 mg/kg, 9 mg/kg, 8 mg/kg, 7 mg/kg, 6 mg/kg, 5 mg/kg, 4 mg/kg, 3 mg/kg, 2 mg/kg, 1 mg/kg, 0.9 mg/kg, 0.8 mg/kg, 0.7 mg/kg, 0.6 mg/kg, 0.5 mg/kg, 0.4 mg/kg, 0.3 mg/kg, 0.2 mg/kg, or 0.1 mg/kg. In some embodiments a therapeutically effective amount of a binding agent is between about 0.1 mg/kg to 500 mg/kg, or between about 1 mg/kg and about 300 mg/kg. Volumes suitable for intravenous administration are well known.

A pharmaceutical composition comprising an amount or dose of a binding agent can, if desired, be provided in a kit, pack or dispensing device, which can contain one or more doses of a binding agent. The pack can for example comprise metal or plastic foil, such as a blister pack. The pack or dispenser device can be accompanied by instructions for administration. The pack or dispenser can also be accompanied with a notice associated with the container in a form prescribed by a governmental agency regulating the manufacture, use, or sale of pharmaceuticals, which notice is reflective of approval by the agency of the form of the drug for human or veterinary administration. Such notice, for example, can be the labeling approved by the U.S. Food and Drug Administration for prescription drugs, or the approved product insert.

In some embodiments a kit or pack comprises an amount of a binding agent sufficient to treat a subject for 1 day to 1 year, 1 day to 180 days, 1 day to 120 days, 1 day to 90 days, 1 day to 60 days, 1 day to 30 days, or any day or number of days there between, 1-4 hours, 1-12 hours, or 1-24 hours.

A kit optionally includes a product label or packaging inserts including a description of the components or instructions for use in vitro, in vivo, or ex vivo, of the components therein. Exemplary instructions include instructions for a diagnostic method, treatment protocol or therapeutic regimen. In certain embodiments, a kit comprises packaging material, which refers to a physical structure housing components of the kit. The packaging material can maintain the components sterilely, and can be made of material commonly used for such purposes (e.g., paper, corrugated fiber, glass, plastic, foil, ampules, vials, tubes, etc.). Product labels or inserts include “printed matter,” e.g., paper or cardboard, or separate or affixed to a component, a kit or packing material (e.g., a box), or attached to an ampule, tube or vial containing a kit component. Labels or inserts can additionally include a computer readable medium, optical disk such as CD- or DVD-ROM/RAM, DVD, MP3, magnetic tape, or an electrical storage media such as RAM and ROM or hybrids of these such as magnetic/optical storage media, FLASH media or memory type cards. Product labels or inserts can include identifying information of one or more components therein, dose amounts, clinical pharmacology of the active ingredient(s) including mechanism of action, pharmacokinetics (PK) and pharmacodynamics (PD). Product labels or inserts can include information identifying manufacturer information, lot numbers, manufacturer location, date, information on an indicated condition, disorder, disease or symptom for which a kit component may be used. Product labels or inserts can include instructions for the clinician or for a subject for using one or more of the kit components in a method, treatment protocol or therapeutic regimen. Instructions can include dosage amounts, frequency or duration, and instructions for practicing any of the methods, treatment protocols or therapeutic regimes set forth herein. Kits of the invention therefore can additionally include labels or instructions for practicing any of the methods and uses of the invention described herein. Product labels or inserts can include information on potential adverse side effects and/or warnings.

In certain embodiments, a kit comprises one or more controls having a known amount of CotH3. In some embodiments, a kit comprises cells expressing CotH3. The cells in the kit can be maintained under appropriate storage conditions until the cells are ready to be used.

In some embodiments, a kit is a diagnostic kits comprising a binding agent. A binding agent comprised in a diagnostic kit can take any suitable form. In some embodiments, a diagnostic comprises a binding agent and a detectable label. In certain embodiments, for example, a diagnostic kit comprises or consists of a stick test, including necessary reagents to perform the method of the invention and to produce, for example, a colorimetric result which can be compared against a color chart or standard curve. A diagnostic kit can also comprise components necessary for detecting a binding agent that specifically binds to CotH3, for example a secondary antibody.

### EXAMPLES

### Example 1—Generation of Mouse Anti-CotH3 Monoclonal Antibody

A mouse monoclonal antibody having binding specificity for CotH3, and designated clone C2 was produced by immunization of a mouse with a peptide having the sequence MGQTNDGAYRDPTDNNK (SEQ ID NO:34). The amino acid sequence of SEQ ID NO:34 highly conserved among Mucorales genera and species thereof and is present in the CotH3 protein of Rhizopus oryzae, Lichtheimia corymbifera, Cunninghamella bertholetiae, and R. mirosporus. The monoclonal antibody (C2) was determined to be an IgG1 kappa isotype and bound specifically to both Mucorales CotH3 and CotH2, as the peptide of SEQ ID NO:34 is conserved 100% in both CotH3 and CotH2. The affinity of the purified 6E11A8 mAb for CotH3 was approximately 40 nM.

### Cloning and Sequencing of Mouse Monoclonal Antibody C2

A reverse primer based on the mouse kappa light chain constant region was used for 5′-RACE PCR to amplify the light chain variable region. A reverse primer based on the mouse heavy chain constant region was used for 5′-RACE PCR to amplify the heavy chain variable region. The amplified PCR products were cloned into a TOPO vector for sequencing analysis.

Nucleic acid sequences of the heavy chain variable regions of C2 are shown in Example 2 and the corresponding conceptually translated amino acid sequences for the heavy chain variable regions and heavy chain CDRs of C2 are shown in Tables 3 and 4. There were some sequence discrepancies between a first nucleic acid sequence obtained for the light chain variable regions of C2 and a second nucleic acid sequence later obtained for C2 (compare C2-6E11A8 to C2-AdvantGen, Tables 1 and 2). Additional sequencing and studies were performed, including N-terminal protein sequencing by Edman degradation, which suggested that the C2-AdvantGen nucleic acid sequence of the light chain variable region of C2 was more accurate correct. Nucleic acid sequences of the light chain variable regions of C2 are shown in Example 2 and the corresponding conceptually translated amino acid sequences for the light chain variable regions and light chain CDRs of C2 are shown in Tables 1 and 2.

### Humanization of the C2 Murine Antibody

Alignments of the murine heavy chain (HC) and light chain (LC) variable regions of C2 with human germline antibody variable regions were performed to identify the best frameworks as acceptors for grafting the mouse C2 mAb VH and VL CDRs. Based on the alignment analysis, the human VH4-59*01 was selected for the HC humanization and the A18 was selected for the LC humanization. The gene encoding the designed grafted humanized variant was custom synthesized by Bio Basic (Ontario, Canada).

Two chimeras and one grafted humanized IgG were then constructed comprising: 1) the cloned variable heavy chain of C2 fused with human IgG1 constant region coupled with a) the 6E11A8 variable kappa region fused to the human kappa constant region (Chimera 1); 2) the cloned variable heavy chain of C2 fused with human IgG1 constant region coupled with b) the AvantGen cloned variable kappa region fused to the human kappa constant region (Chimera 2); and 3) a grafted humanized version where the 6E11A8 variable heavy chain CDRs and the 6E11A8 variable light chain CDRs were respectively grafted into the human framework regions of human IgG1 heavy chain (VH4-59*01) and human kappa light chain (A18). For this, the DNA encoding the variable heavy chain regions was cloned into BamHI and Apa I sites of AvantGen's pcDNA3.4-IgG1 vector and the sequences were confirmed. The entire heavy chain sequences of the mouse-human chimeras and grafted humanized heavy chains are shown in Tables 3 and 4. The light chain variable regions for these constructs were cloned into the Afl II and Acc65 I sites of AvantGen's pcDNA3.4-kappa vector and the sequences were confirmed. The entire light chain sequences of the two mouse-human light chain chimeras and humanized grafted light chains are shown in Tables 3 and 4.

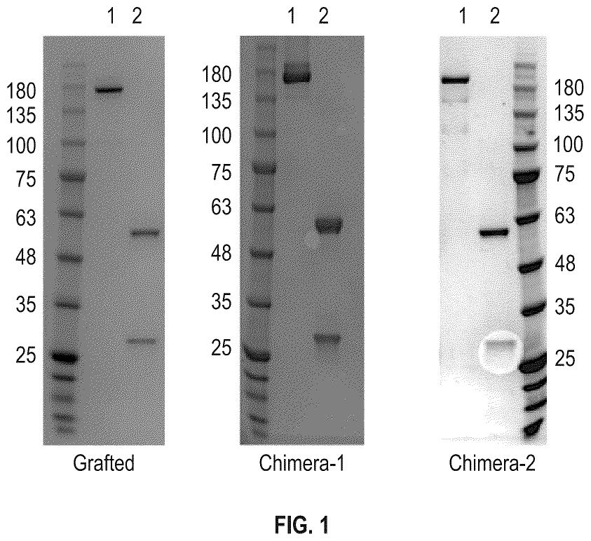

Large scale plasmid preparations were prepared and used for transfection of hamster ExpiCHO cells. Secreted antibody was purified from the culture medium 10 days post transfection using Protein A affinity chromatography. Purified antibody was analyzed by SDS-PAGE gel to determine the purity of the antibody, which is estimated to be greater than 95% pure (FIG. 1). The antibody concentration was determined by UV absorbance at 280 nm.

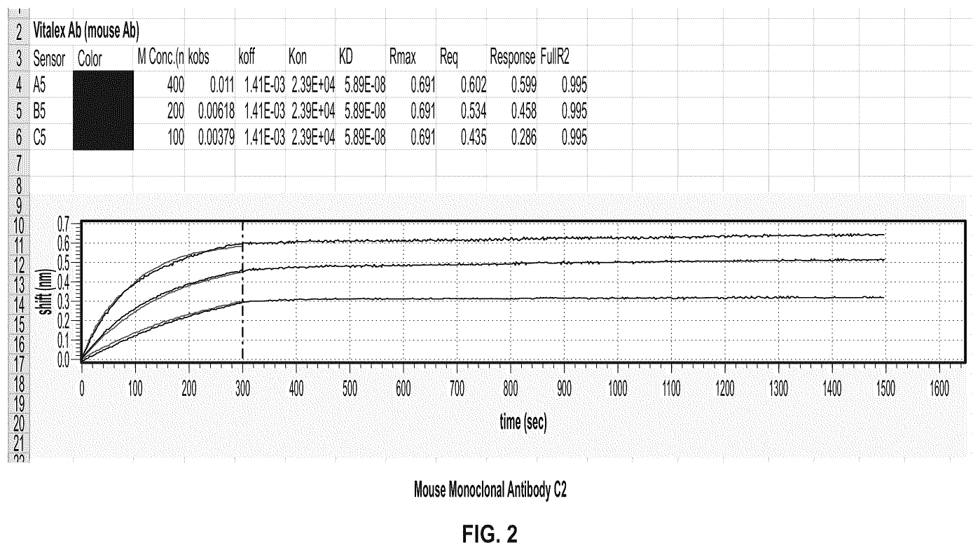

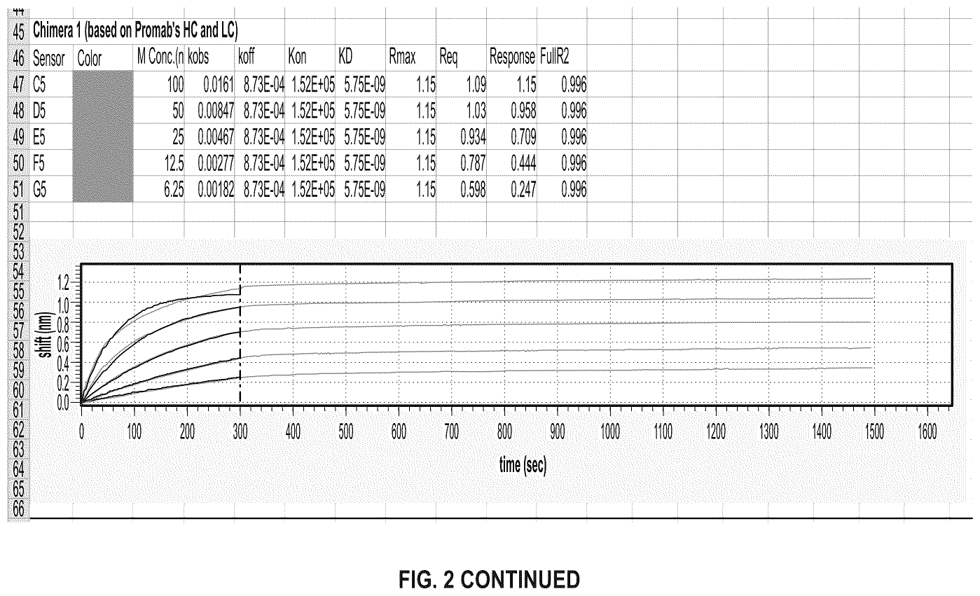

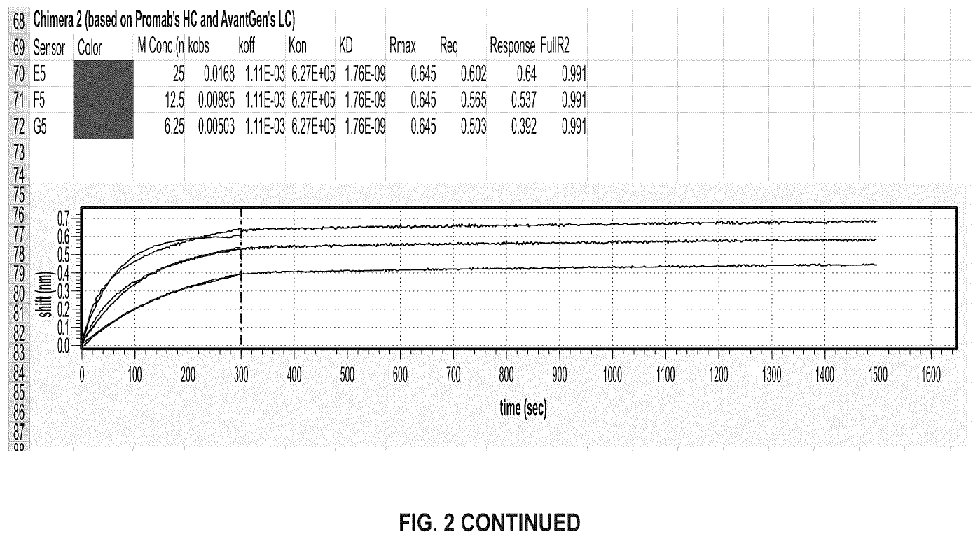

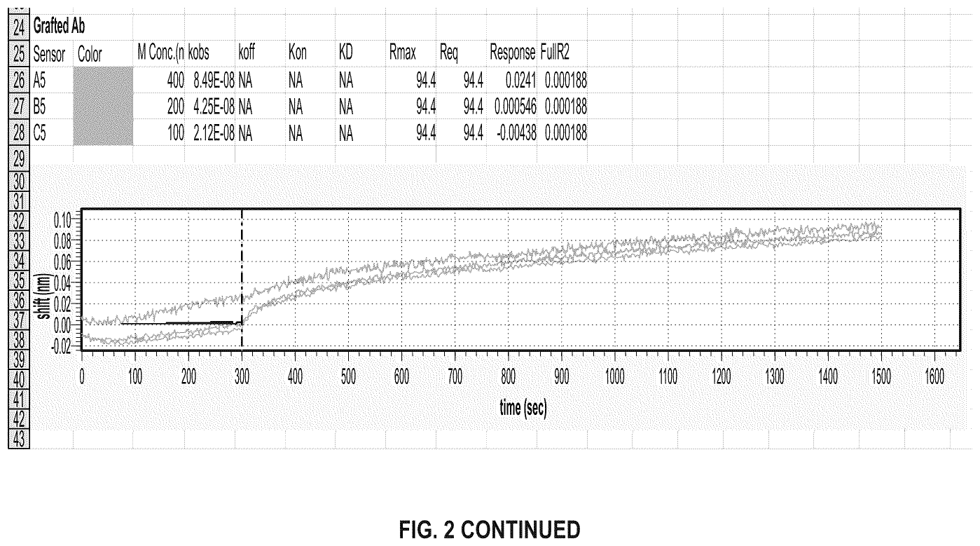

The purified preparations were then used to assess the affinity of each clone for the CotH3 antigen by biolayer interferometry using Gator system (ProbeLife). Under the assumption that the CotH3 antigen provided by Vitalex behaves as a normal soluble monomeric protein, a Protein G coated sensor probe (ProbeLife, catalog #160006) was loaded with the bivalent antibody test clone at a concentration of 5 μg/ml in ProbeLife's kinetic buffer (PBS pH 7.4, 0.02% BSA, 0.002% Tween-20, 0.005% NaN₃). A serial dilution of CotH3 antigen of 400, 200, 100, 50, 25, 12.5, 6.25 nM and buffer only (0 nM) was added to the sensors. All reactions were performed at 25° C. The values are chosen when three or more concentrations yield the same kinetic values. The original murine mAb was provided by Vitalex as the reference. The raw data and Gator software deduced kinetics data are shown in FIG. 2 and a summary of the Kₒₙ, K_{off} and K_{D} values are shown in Table 5.

TABLE 5

| Clone | K_{D} (nM) | Kₒₙ (M⁻¹s⁻¹) | Koff (S⁻¹) | Rmax | R2 |
|---|---|---|---|---|---|
| mAb | 58.9 | 2.39E+04 | 1.41E−3 | 0.69 | 0.995 |
| Chimera-1 | 5.7 | 1.52E+05 | 8.73E−04 | 1.15 | 0.996 |
| Chimera-2 | 1.8 | 6.27E+05 | 1.11E−03 | 0.65 | 0.991 |
| Grafted- | ND | ND | ND | | 0.00019 |
| humanized | | | | | |

The data showed that the original murine antibody exhibited a K_{D} value of 58.9 nM, however Chimera-1 showed an approximately 10-fold improvement and Chimera-2 showed a 33-fold improvement in affinity over the parental mouse mAb.

In contrast, the grafted humanized variant exhibited no detectable binding above the noise of a slowly increasing baseline signal.

### Modeling of the Murine Compared to Grafted Humanized Variable Regions and Design of the Humanized Variants.

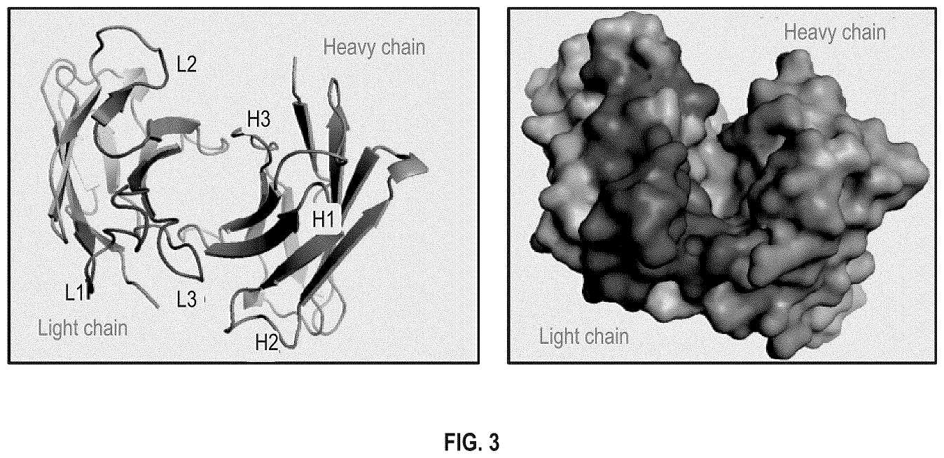

As the first step towards restoring binding activity, computer modeling and analysis were performed for the mouse antibody and humanized grafted antibody with the selected human antibody frameworks and mouse antibody CDRs. PDB structures 1XGP (heavy chain) and 2W60 (light chain) was chosen as homology templates for the murine variable regions and 4LKX for the human framework with the grafted murine CDRs (FIG. 3). A structural assessment of the homology models was then carried out—models were visually assessed using Pymol (Schrödinger, Inc.) and residue sequence/structure commonalities and differences between the three models (mouse and humanized models with either 6E11A8 or AvantGen light chains) were used to guide where backmutations might be warranted. For the light chain, the residues which could potentially impact the folding of the CDRs within the human A18 framework fell into 3 groups as summarized in Table 6, which shows a summary of the 3 sets of framework residues for each chain (light chain (upper set); heavy chain (lower set)) that could impact the binding pocket presented by the 3 CDRs and are candidates for back-mutations from the human to the murine residue.

TABLE 6

| | | |
|---|---|---|
| Mutations* | Grouping | Comments |

Light chain mutations

| | | |
|---|---|---|
| S53K, R54L, | S53K, R54L, | CDRL2 conformation forced to be |
| F55D | F55D | different in graft chain due to presence |
| | | of L54R mutation. |
| | | IDT name: Vit_Hum_BM_LC1 |
| Y34N, Y36L | Y34N, Y36L + | Contract residue or influences contact |
| | S53K, R54L, | residue orientation W89 or L89 should |
| | F55D | work based on LC1 and LC2 sequences. |
| | | IDT name: Vit_Hum_BM_LC2 |
| L46R | L46R + Y34N, | Potential role in binding or stabilizing |
| | Y36L S53K, | CDR conformations. |
| | R54L, F55D. | IDT name: Vit_Hum_BM_LC3 |

Heavy chain mutations

| | | |
|---|---|---|
| V71R | V71R | Possibly influences CDRH2 |
| | | conformation. |
| | | IDT name: Vit_Hum_BM_HC1 |
| W47H, N58F | W47H, N58F + | H47 is part of the Vernier zone. H58 |
| | V71R | pairs with H47 w.r.t. space-filling. |
| | | IDT name: Vit_Hum_BM_HC2 |
| S35N | S35N + W47H, | Possible contact residues. |
| | N58F, V71R | IDT name: Vit_Hum_BM_HC3 |

*Kabat numbering

In order of theoretical impact these groupings were as follows. 1) Residues S53, R54 and F55 at the beginning of framework 3 of the human chain adjacent to CDR2 were predicted to force a different conformation of CDRL2 to that conferred by the KLD residues at these positions in the murine VK, therefore the first LC variant, named Vit_Hum_BM_LC1, was constructed with the K53, L54 and D55 backmutations at these positions. 2) Residues Y34 and Y36 at the beginning of framework 2 of the human acceptor framework represent contact residues which could impact CDR orientation. Therefore, the second LC variant, Vit_Hum_BM_LC2, carried both the S53K, R54L, F55D back-mutations along with the Y34N and Y36L back-mutations. Finally, the L at position 46 in the human framework compared to R in the murine framework at this position was predicted to potentially have a role in binding or stabilizing CDR conformations. Therefore, the third LC variant, named Vit_Hum_BM_LC3, was designed to carry the L46R back-mutation along with the other five S53K, R54L, F55D, Y34N and Y36L back-mutations. Please note that not all residues mentioned above are displayed, since they are either hidden or overlapped with other residues.

For the heavy chain, the V at position 71 in the human VH instead of the R at this position in the murine VH was predicted to be important to maintain the conformation of CDR-H2, therefore, the V71R back-mutation was generated in Vit_Hum_BM_HC1. Then the differences in positions 47, which falls within the Vernier zone of CDR-H2 and its space filling partner at position 58, W47 compares to H47 and N58 compared to F48, were considered to potentially adversely impact the orientation of the CDRs and hence the antigen binding pocket. Therefore, these two back-mutations were added to the V71R mutation to produce the second humanized VH variant, Vit_Hum_BM_HC2. The final residue that was considered to potentially impact the CDR conformation and antigen binding pocket was the S at position 35 which was an N in the murine sequence and a possible antigen contact residue in CDR-H1. Vit_Hum_BM_HC2 was designed to carry all four back mutations, S35N, W47H, N58F and V71R. The amino acid sequences of the three designed humanized LCs (LC1, LC2 and LC3 and HCs (HC1, HC2 and HC3) are shown aligned in Tables 2 and 4. The nucleotide sequences of the complete heavy and light chains are provided in Example 2.

DNA fragments encoding the humanized variants were custom synthesized by Integrated DNA Technologies (IDT) and cloned into AvantGen's antibody expression vectors. Each LC, including the straight grafted variant was paired with each HC variant to give a total of 16 different HC/LC antibody clones. Small-scale plasmid preps were used to transfect ExpiCHO cells and the yield of each antibody clone was estimated using protein G probe capture (versus standard IgG) via the Gator system. A fresh batch of chimera was also produced to serve as the reference control.

### Assessing the Binding Kinetics of the Humanized Antibody Variants Compared to the Reference Chimera to CotH3 Antigen.

The HC and LC variants with the indicated back-mutations and the pairs that were used for antibody production are shown in Table 7.

TABLE 7

| | | HC2 | HC3 | |
|---|---|---|---|---|
| Light chain/ | HC1 | W47H, N58F, | S35N, W47H, | Grafted |
| Heavy chain* | V71R | V71R | N58F, V71R | HC |
| LC1 | [symbol] | [symbol] | [symbol] | [symbol] |
| S53K, R54L, F55D | | | | |
| LC2 | [symbol] | [symbol] | [symbol] | [symbol] |
| Y34N, Y36L, S53K, | | | | |
| R54L, F55D | | | | |
| LC3 | [symbol] | [symbol] | [symbol] | [symbol] |
| Y34N, Y36L, L46R, | | | | |
| S53K, R54L, F55D | | | | |
| Grafted LC | [symbol] | [symbol] | [symbol] | [symbol] |

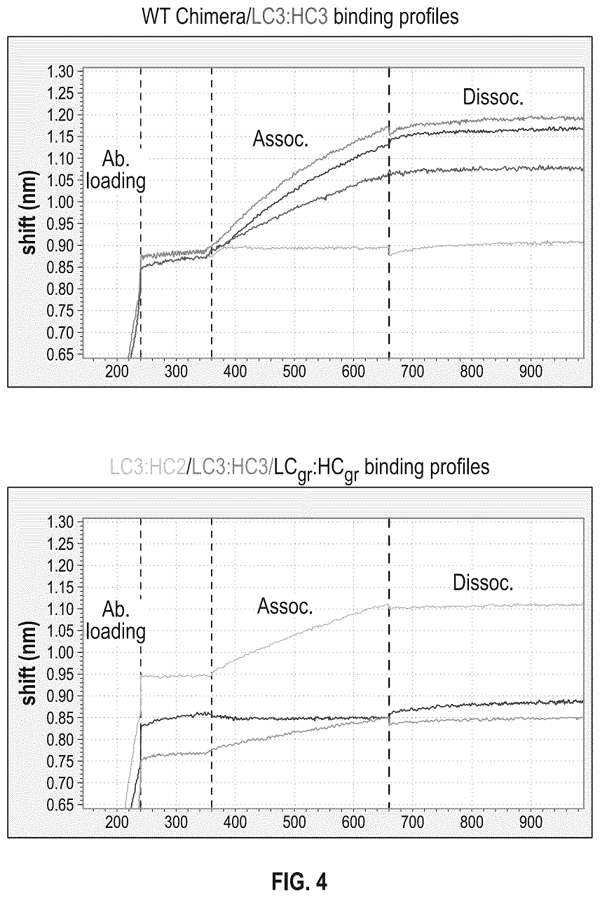

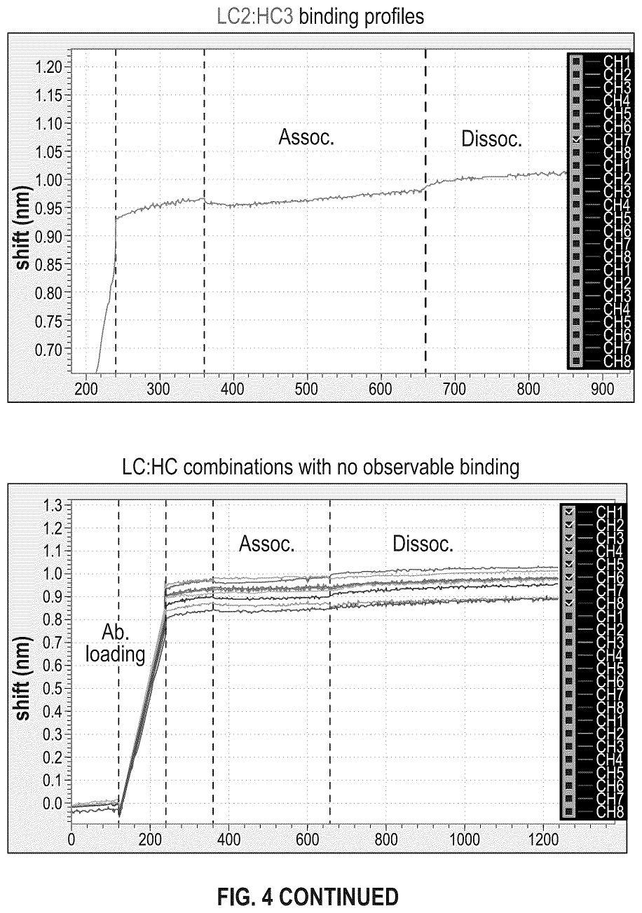

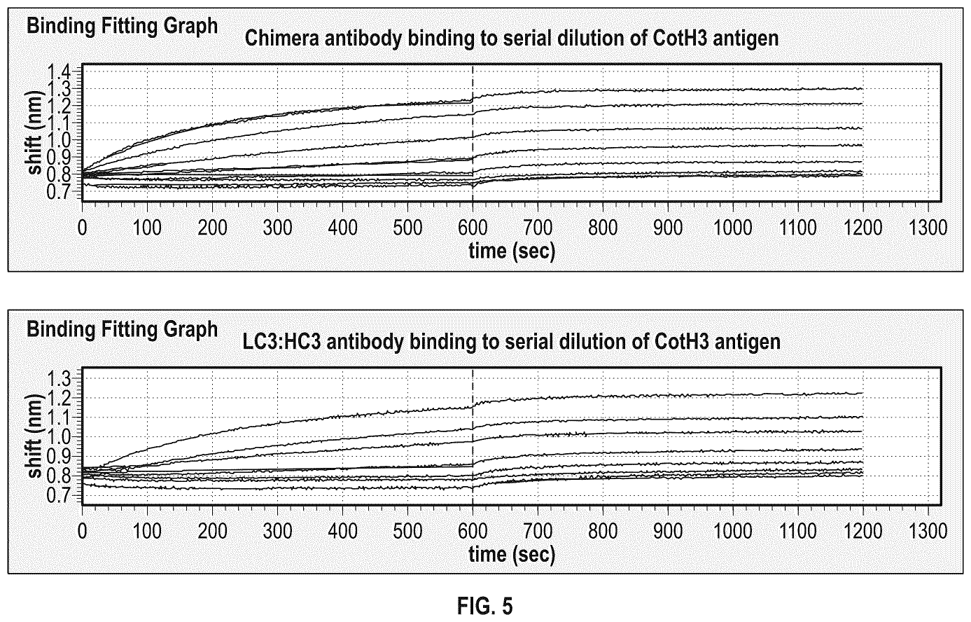

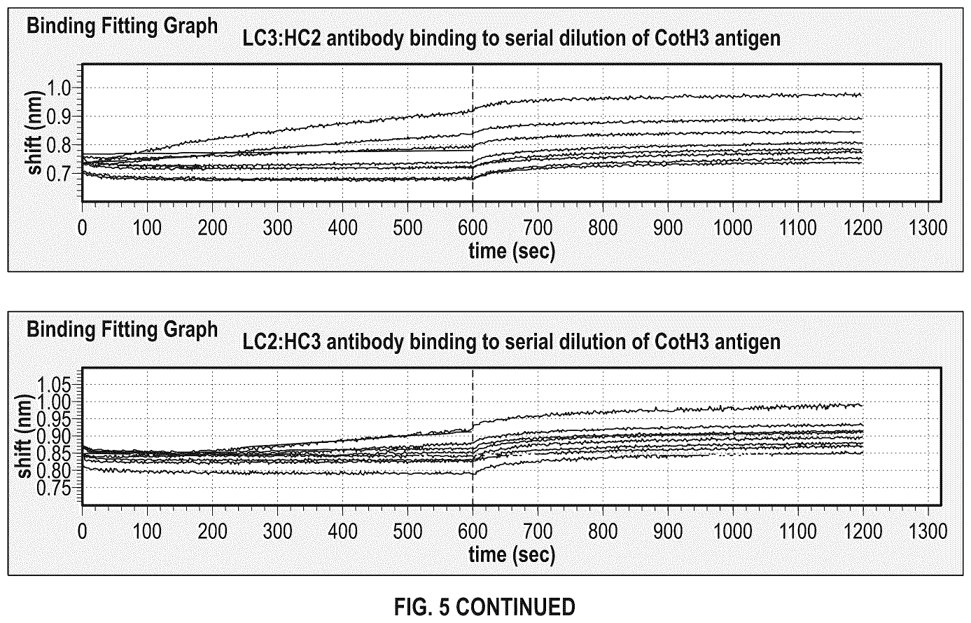

Table 7 shows a Light and heavy chain matrix used for antibody production and the purified antibody tested, via Gator, for binding to CotH3. Murine back-mutations introduced in the context of the grafted antibody are shown for each light/heavy chain. Observed binding to CotH3 is indicated by a tick. The clones in the culture media were then assessed for their binding activity to CotH3 antigen using BLI methodology with the Gator system to determine the best binders. For this, 1 nM antibody was loaded per Protein G sensor in binding buffer (PBS pH 7.4, 0.02% BSA, 0.002% Tween-20) at 30° C. Then 30 nM CotH3 antigen was added and on-rates were monitored for 300 seconds before the dissociation step was initiated and the BLI signal recorded for another 300 seconds. As shown in FIG. 4, only three humanized variants exhibited distinct binding to CotH3 that matched that of the reference chimera, namely LC3:HC3, LC3:HC2 and LC2:HC3. No detectable binding was observed for any of the variants with the grafted LC or LC-1 or the variants with the grafted HC or HC-1 (see bottom right hand panel of FIG. 4). For all three variants and the chimera, no detectable dissociation was observed (FIGS. 4 and 5). Based on the association portion of the traces, the rank order appeared to be chimera>LC3:HC3>LC3:HC2>LC2:HC3. This data set indicates that the first tier of back-mutations for both chains failed to restore CotH3 binding activity. Rather the Y34N and Y36L back-mutations in the LC and the W47H, and N58F back-mutations of the HC were required to restore some antigen binding activity and the further addition of the L46R on the LC and S35N on the HC to further improve binding activity to be closer to that of the chimera.

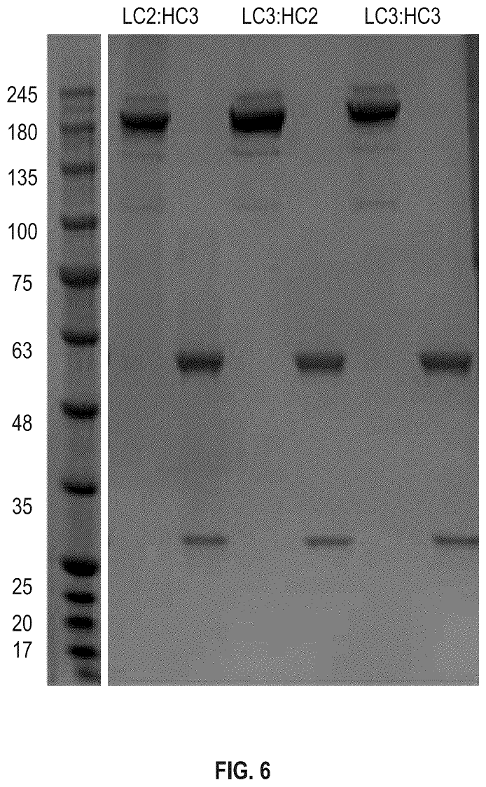

While the data indicated that all three of these variants had binding activity because the Kobs appeared similar to that of the chimera, no definitive KD values could be determined given no detectable dissociation was observed over the 300 second dissociation period. The top three clones and the chimera were purified from the culture media by Protein A chromatography and shown to be endotoxin free. The final yield was >1 mg and by SDS-PAGE the three humanized clones appeared >95% purity (FIG. 6).

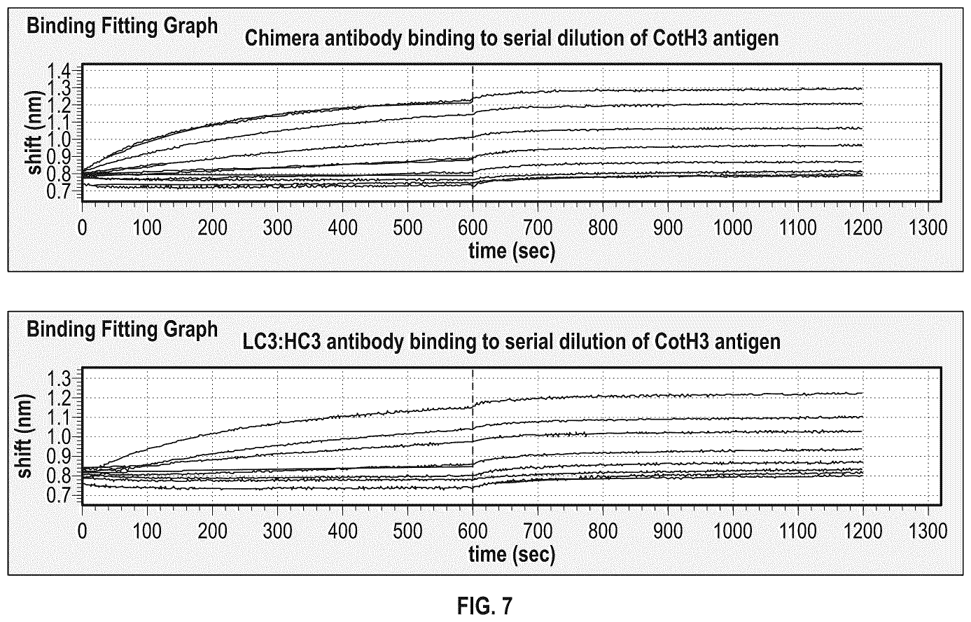

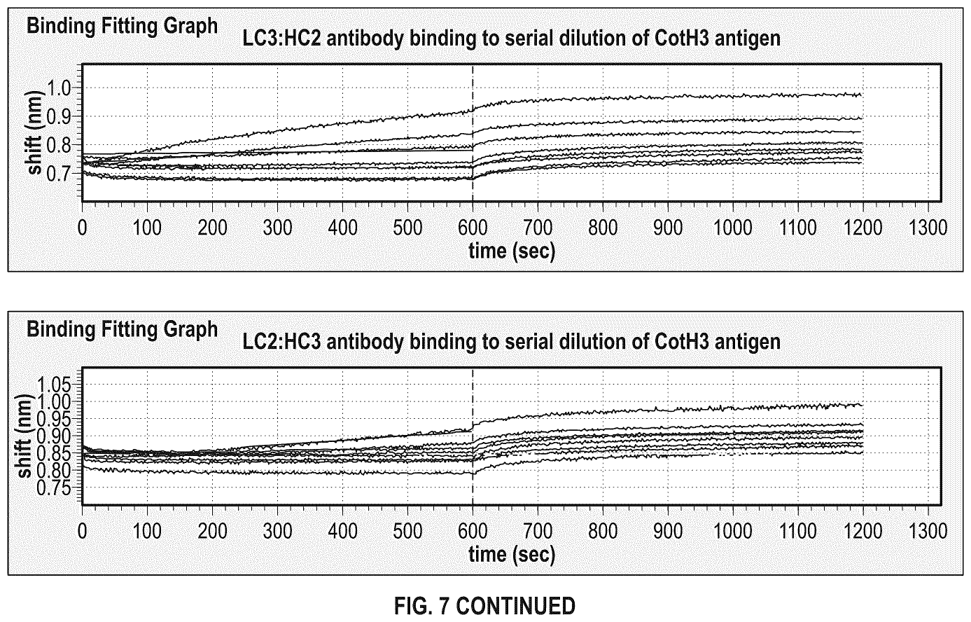

Next the association rates for a serial dilution of CotH3 antigen (60, 30, 15, 7.5, 3.75, 1.875, 0.9375, and 0 nM) was assessed and the antibody-antigen association rates fitted to give Kobs. The binding curves are shown in FIG. 7. Assuming that Koff is equivalent for Chimera vs. LC3:HC3, the data would suggest that the KD value for the LC3:HC3 humanized variant could be approximated to be within 2-fold of the chimera based on AKobs (Table 8).

TABLE 8

| Conc. | Kobs | | | | R² | | | | X² | | | |
|---|---|---|---|---|---|---|---|---|---|---|---|---|
| (nm) | Chim. | L3:H3 | L3:H2 | L2:H3 | Chim. | L3:H3 | L3:H2 | L2:H3 | Chim. | L3:H3 | L3:H2 | L2:H3 |
| 60 | 5.05e−3 | 4.37e−3 | 1.79e−3 | 1.49e−6 | 0.997 | 0.998 | 0.996 | 0.958 | 1.900 | 1.475 | 1.090 | 1.811 |
| 30 | 2.95e−3 | 2.26e−3 | 6.70e−6 | 1.62e−7 | 1.000 | 0.991 | 0.984 | 0.456 | 0.715 | 0.698 | 1.414 | 3.338 |
| 15 | 8.59e−4 | 7.14e−5 | 3.19e−7 | — | 0.995 | 0.987 | 0.405 | — | 1.518 | 1.708 | 3.318 | — |
| 7.5 | 6.12e−7 | 2.13e−7 | — | — | 0.930 | 0.571 | — | — | 2.821 | 3.876 | — | — |

Table 8 shows Kobs determined using Gator software analysis of the association curves at the different CotH3 antigen concentrations for the three selected humanized clones compared to the chimera shown in FIG. 7.

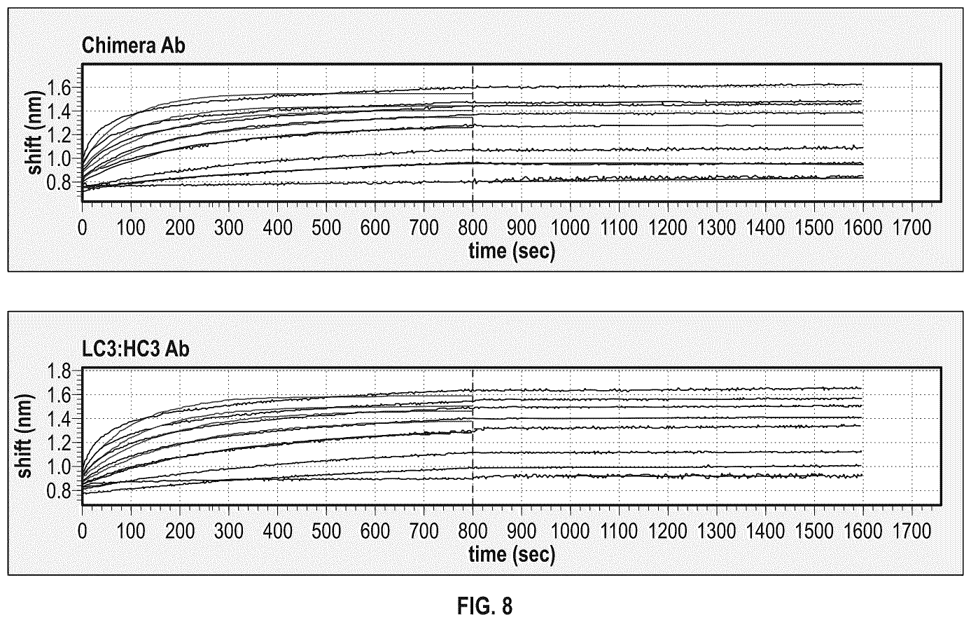

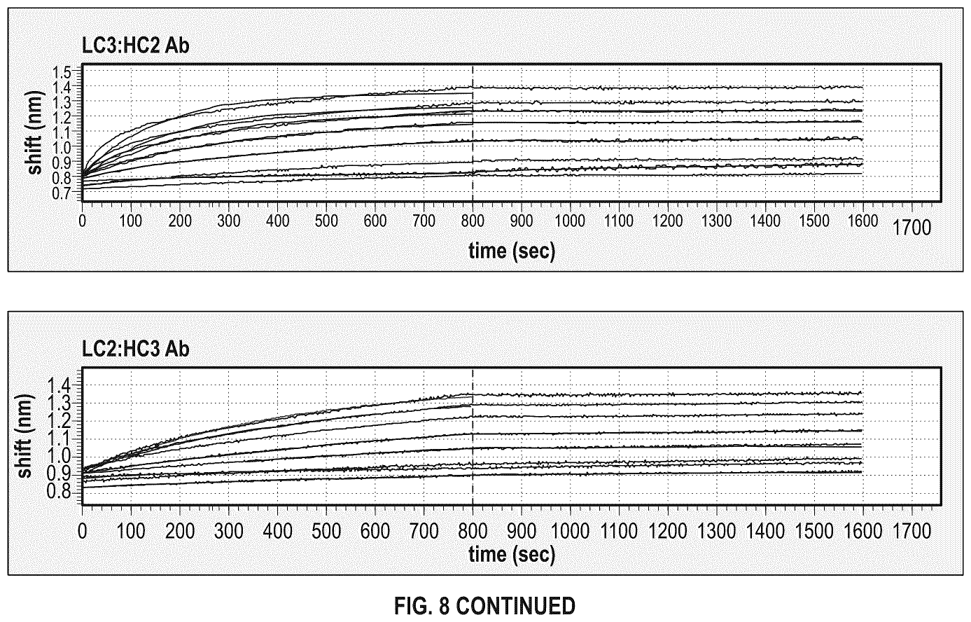

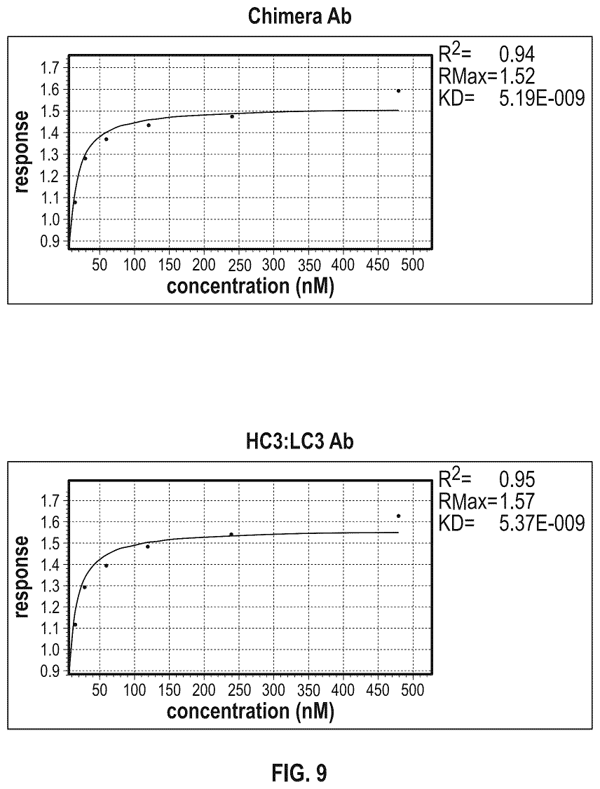

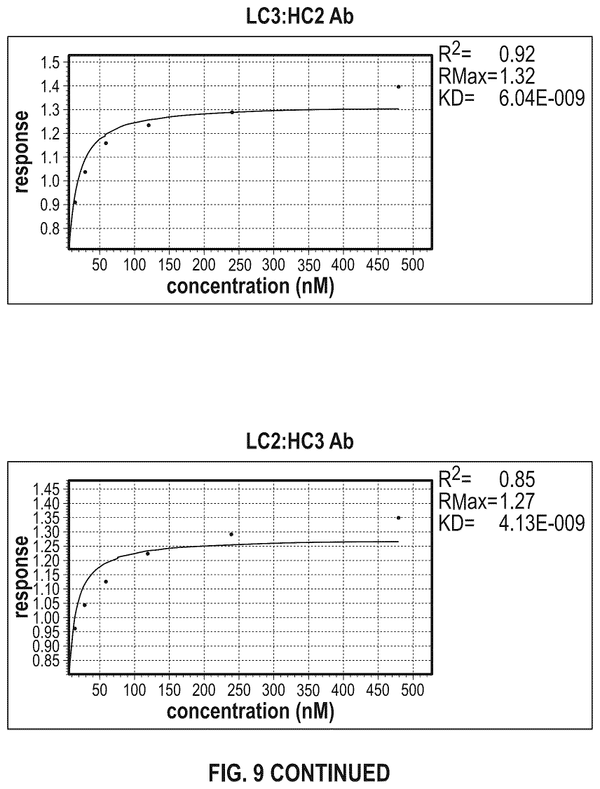

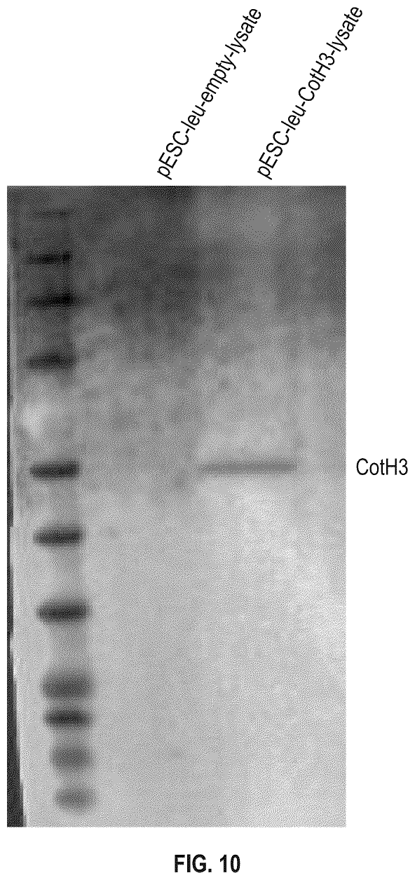

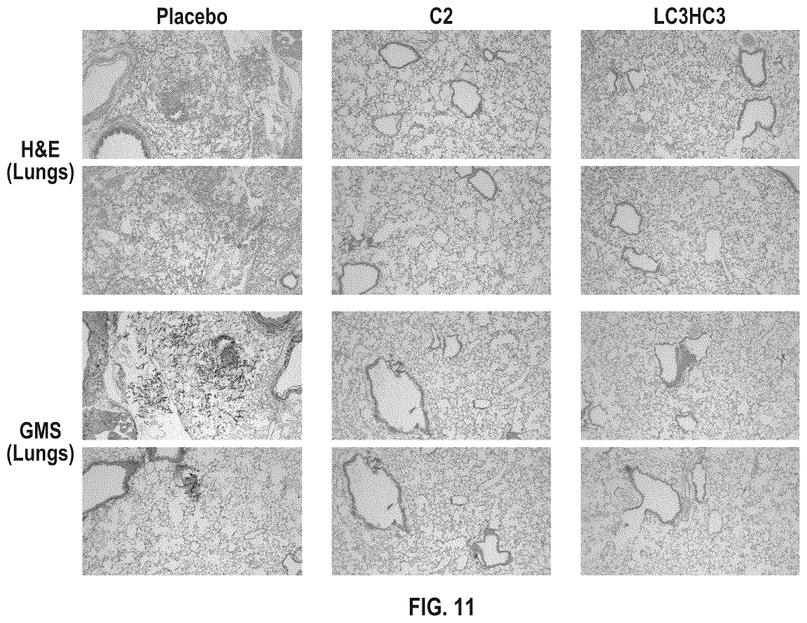

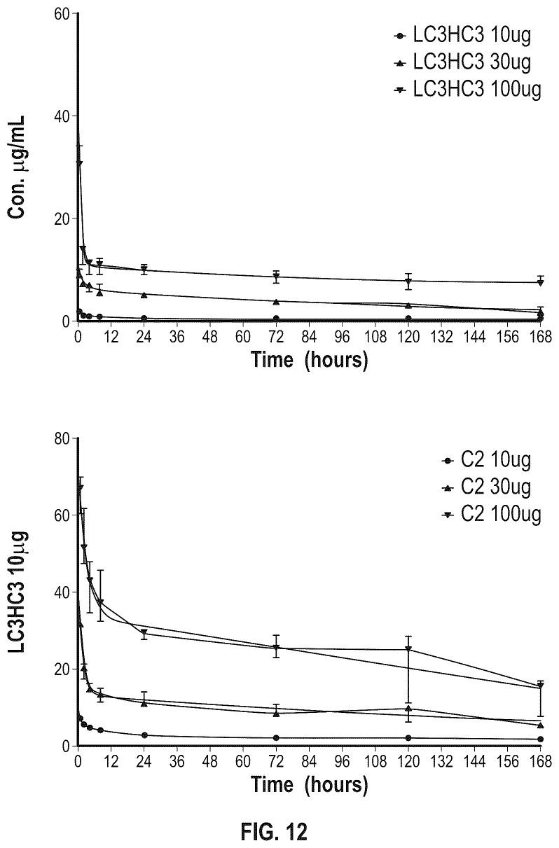

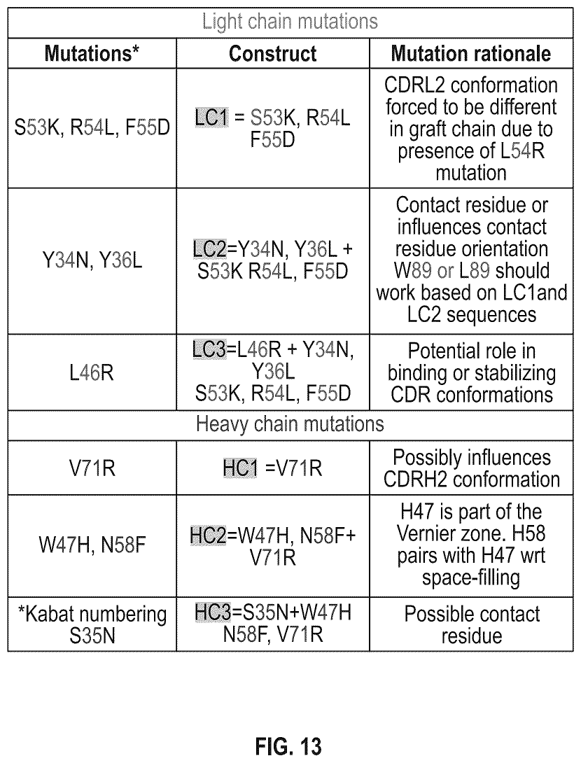

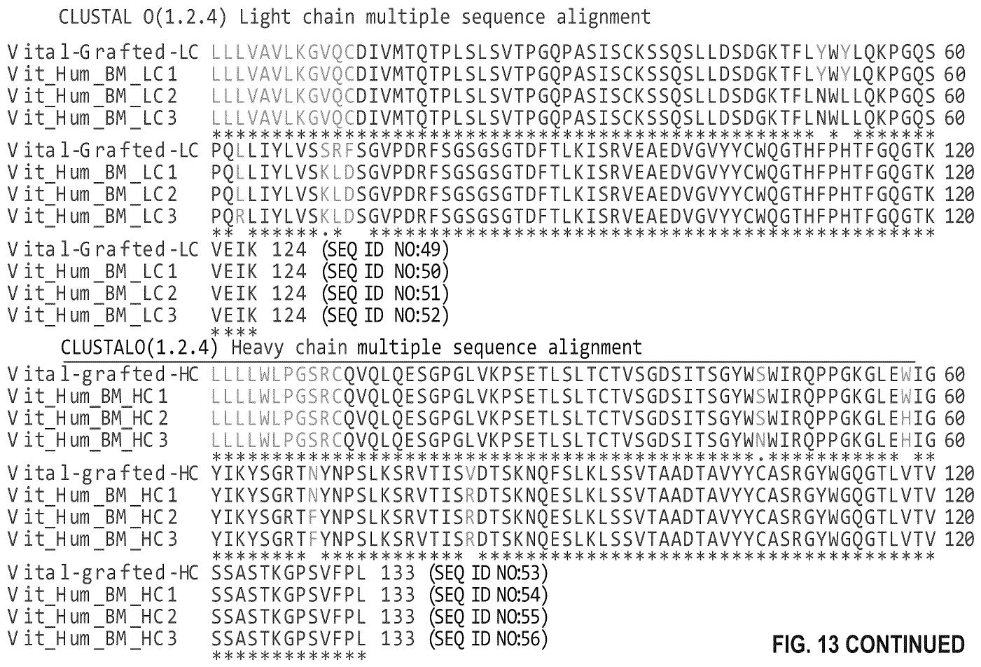

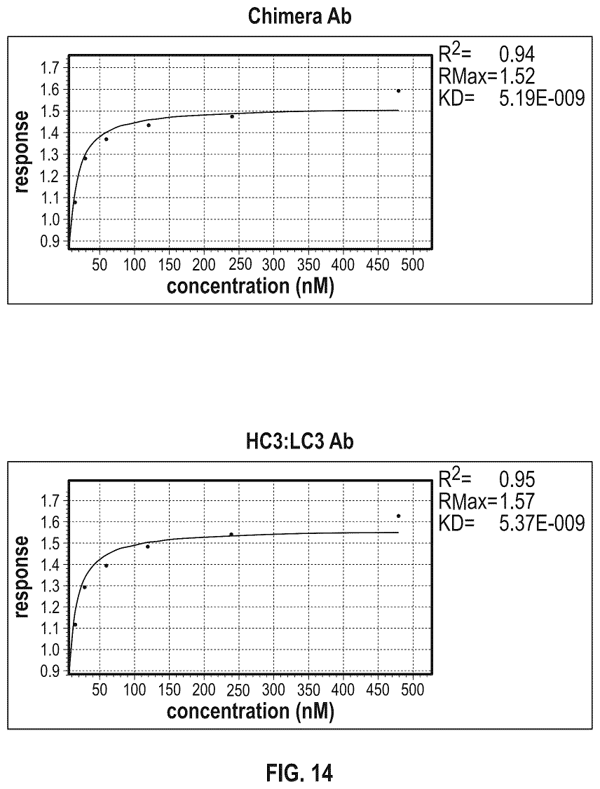

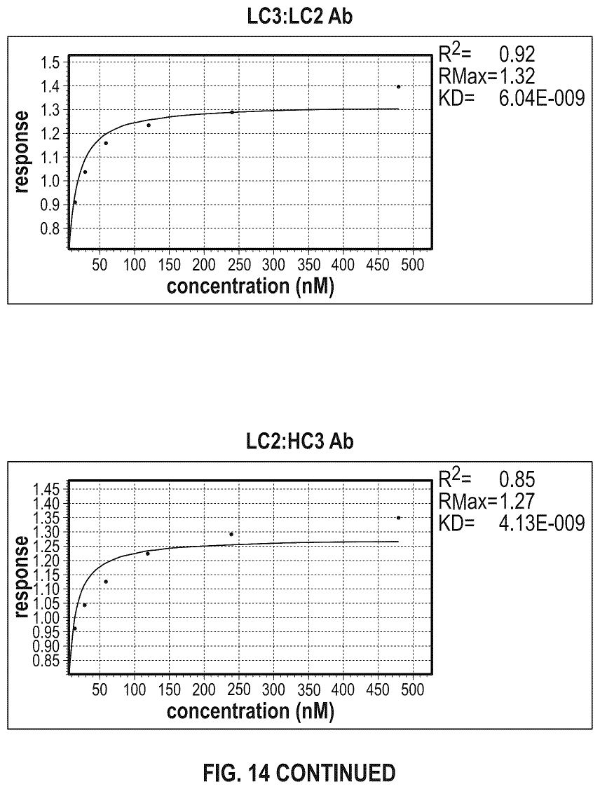

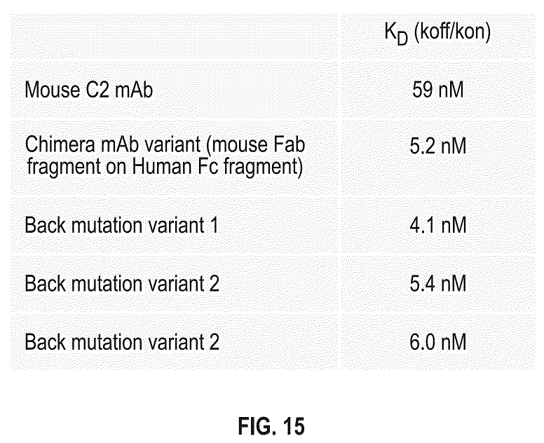

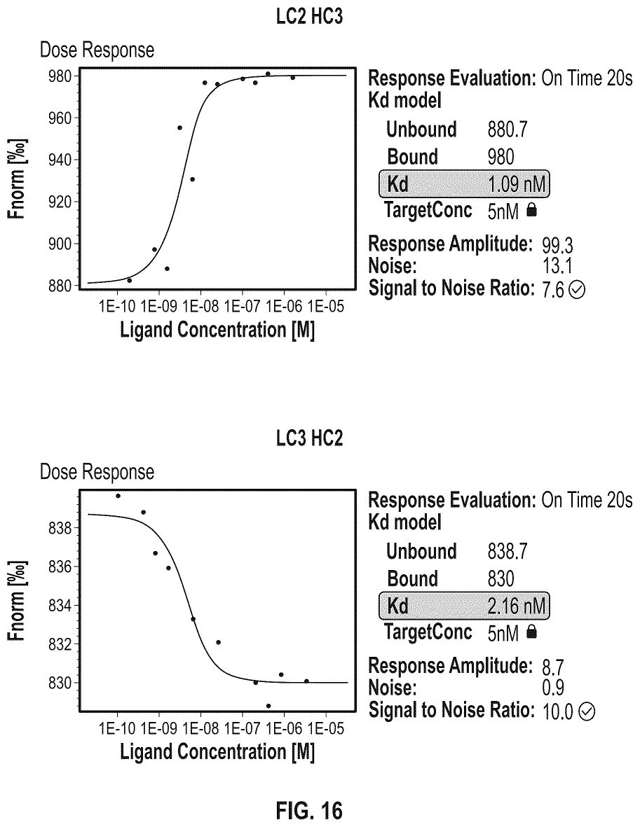

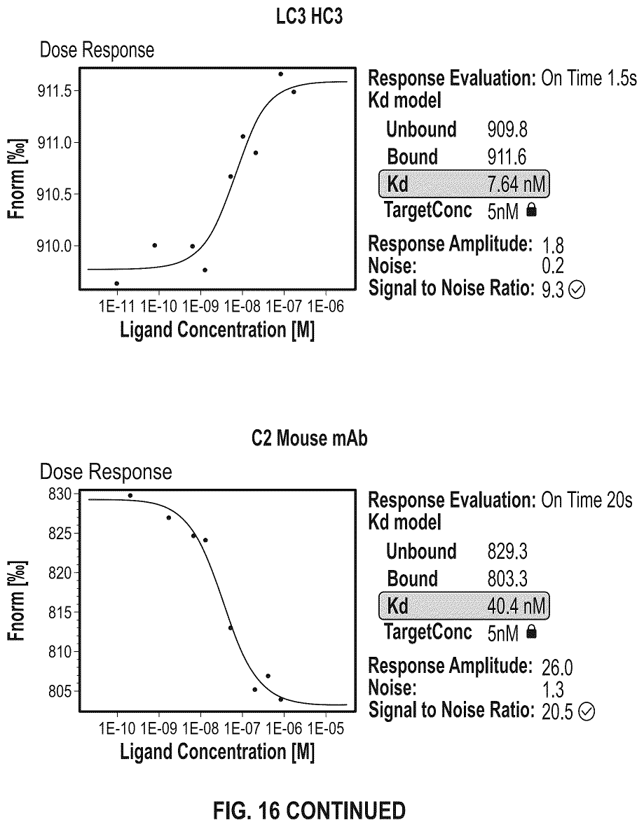

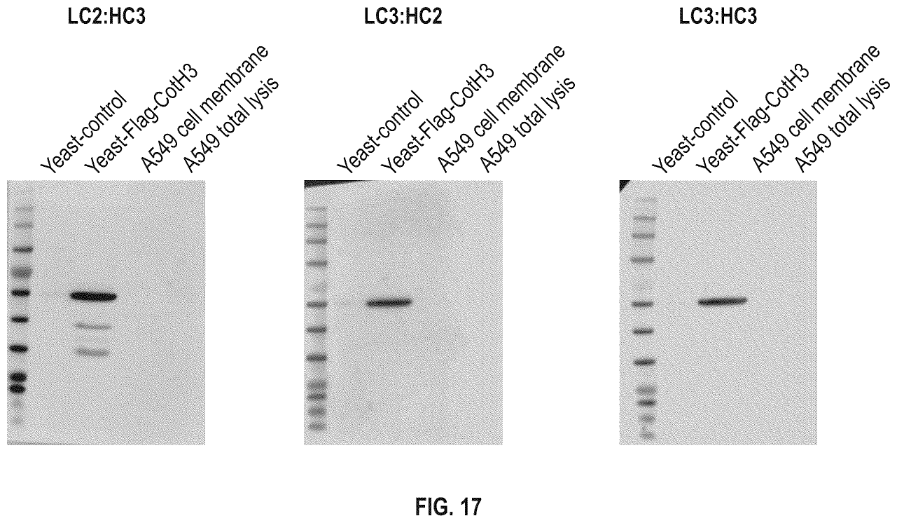

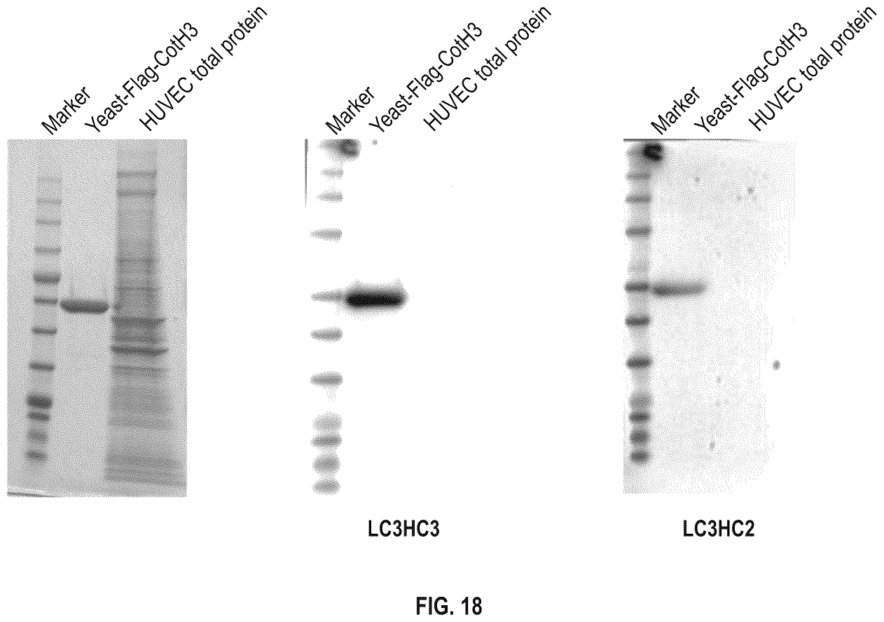

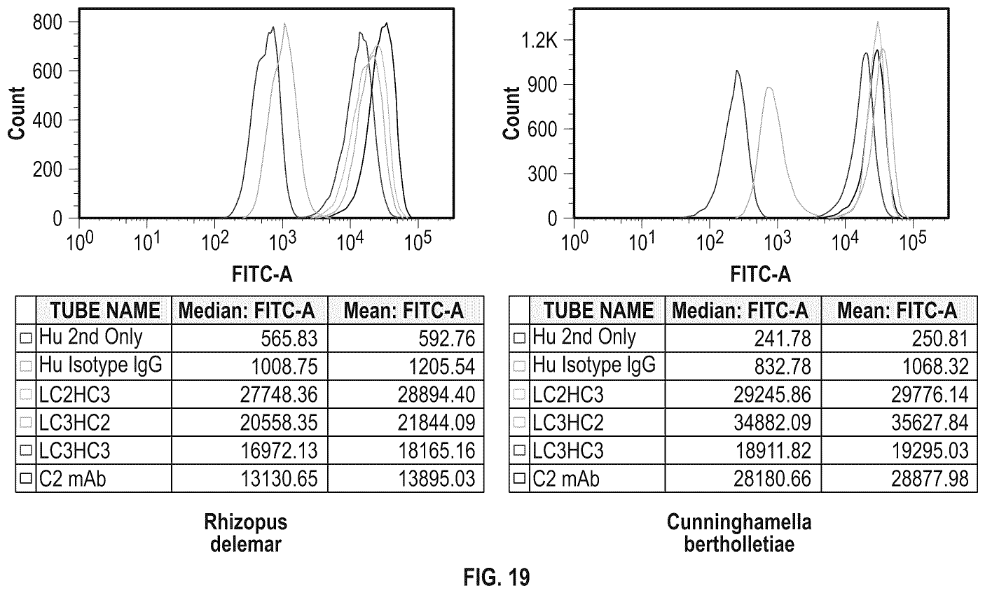

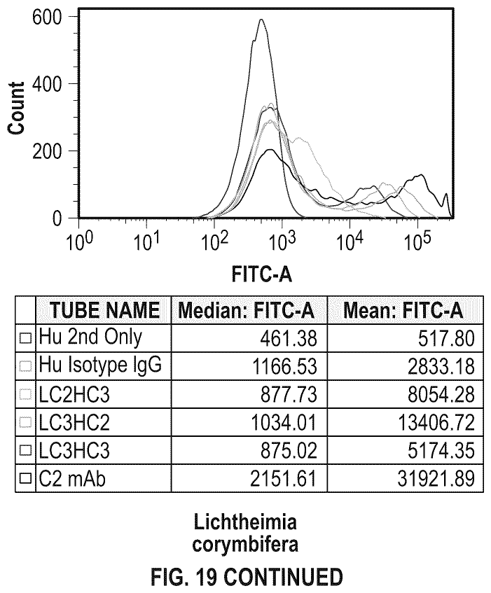

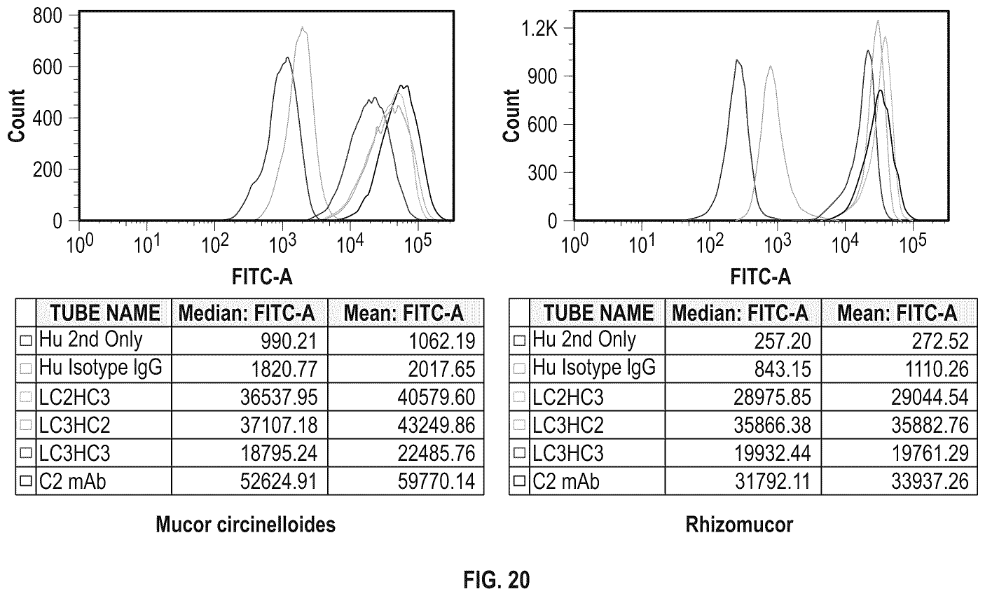

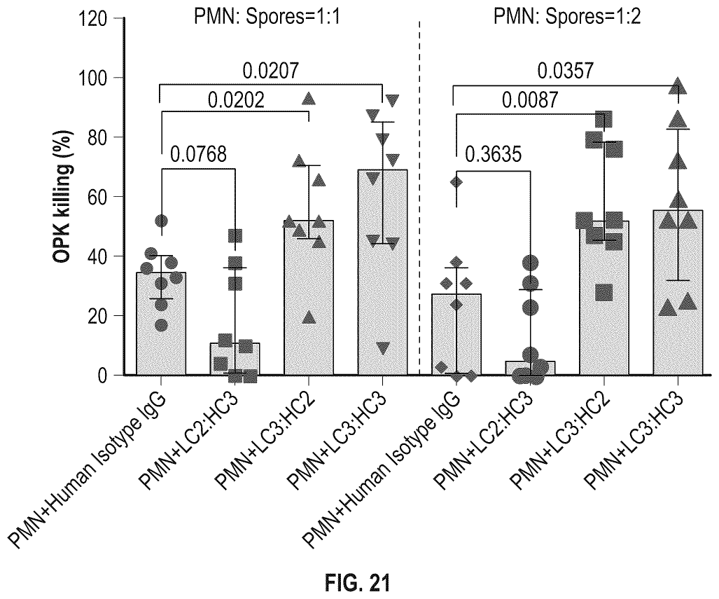

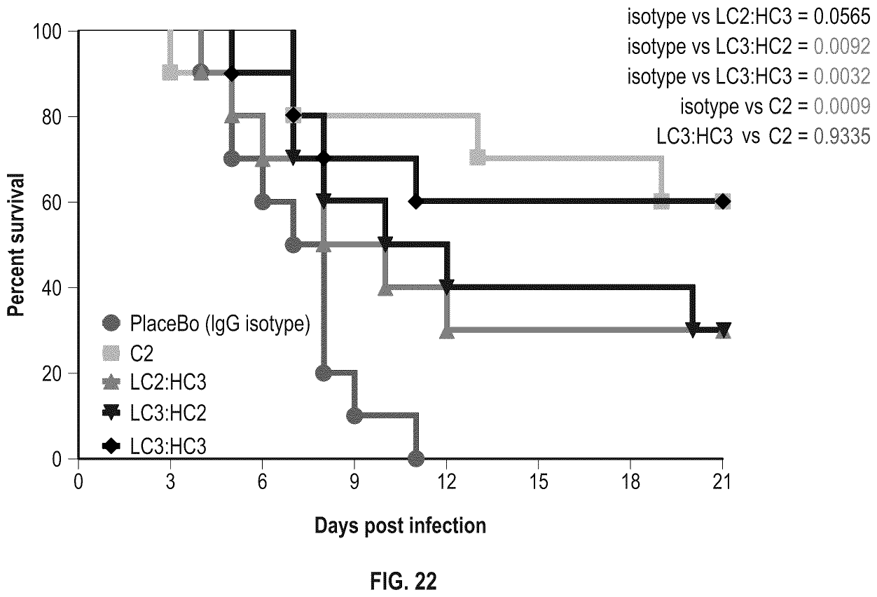

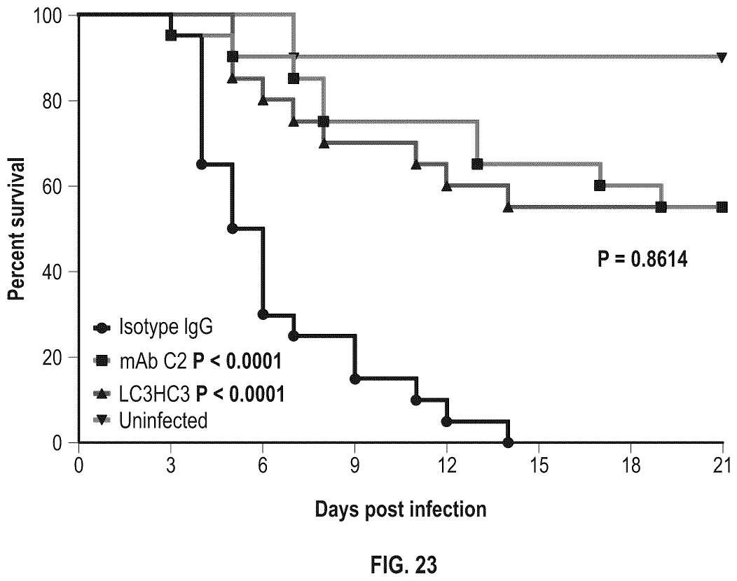

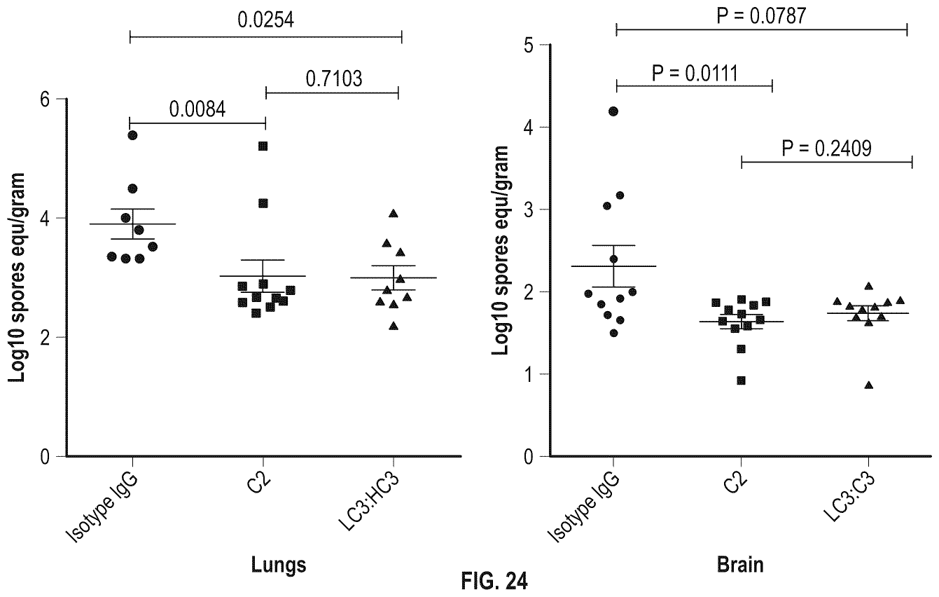

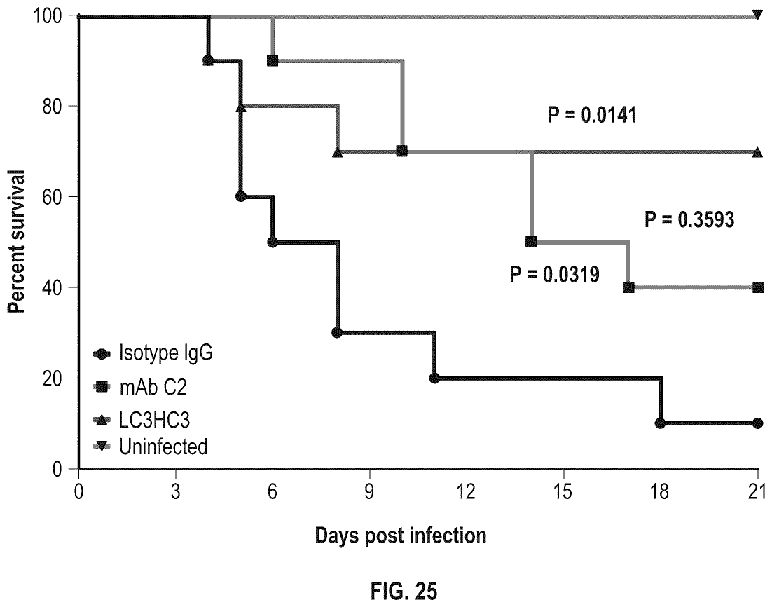

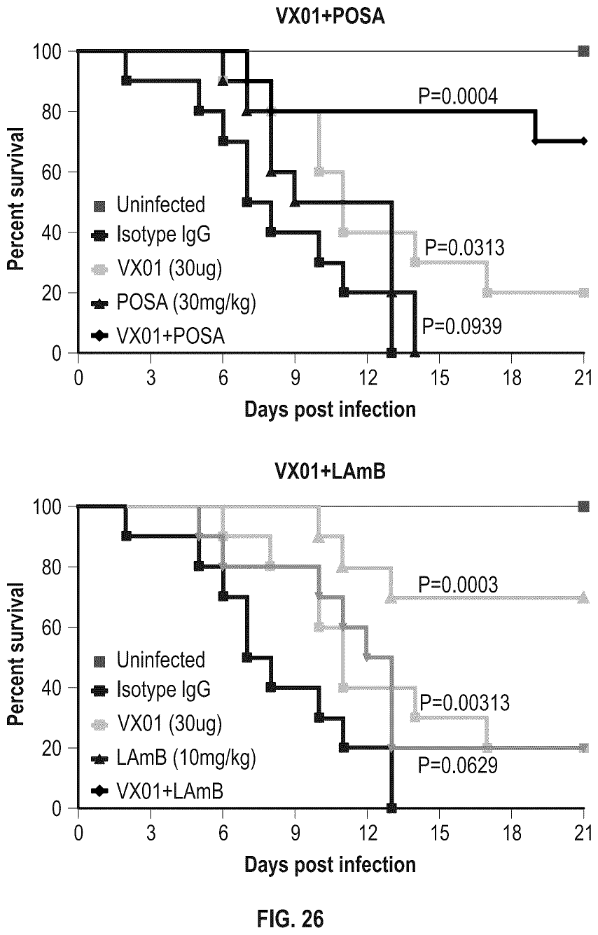

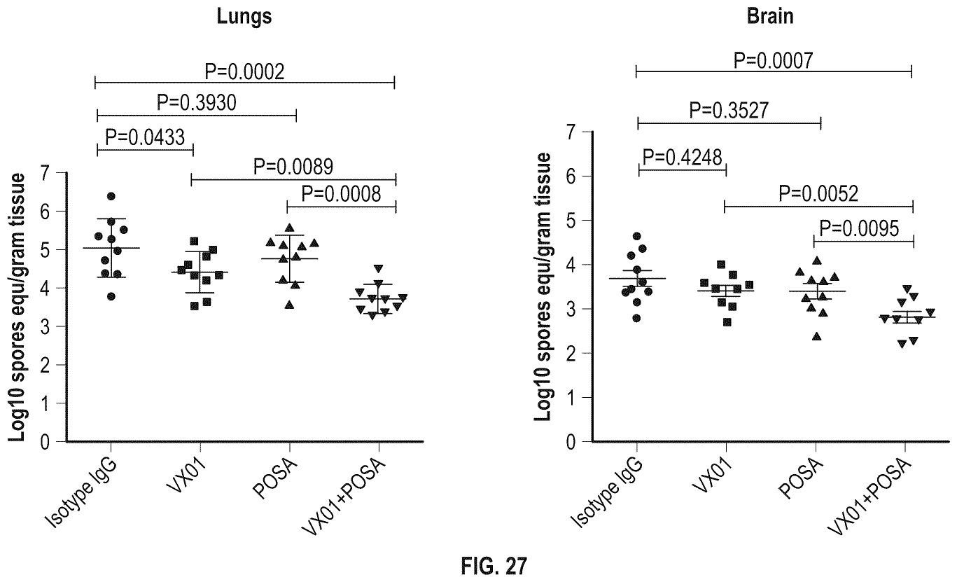

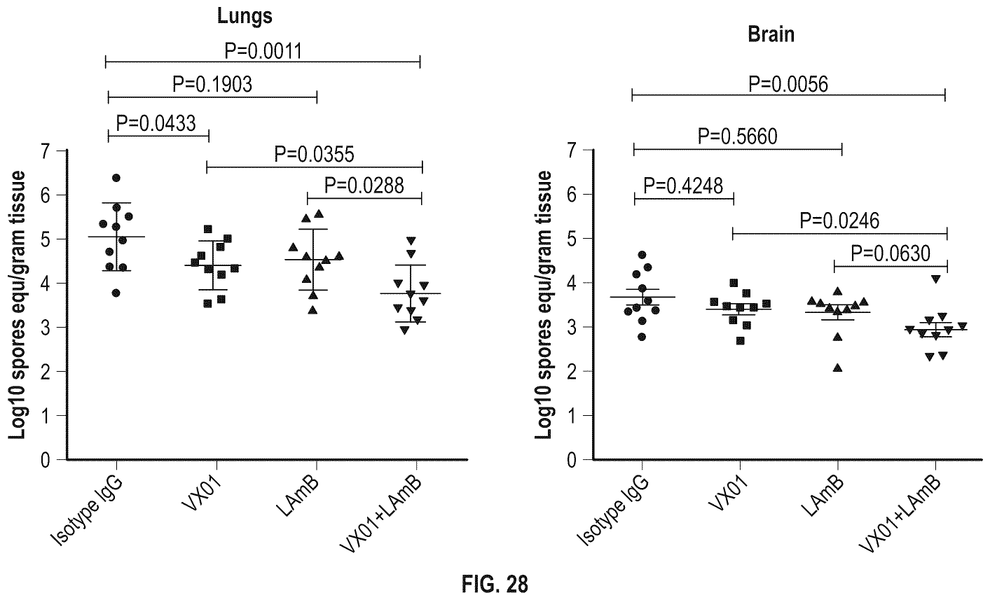

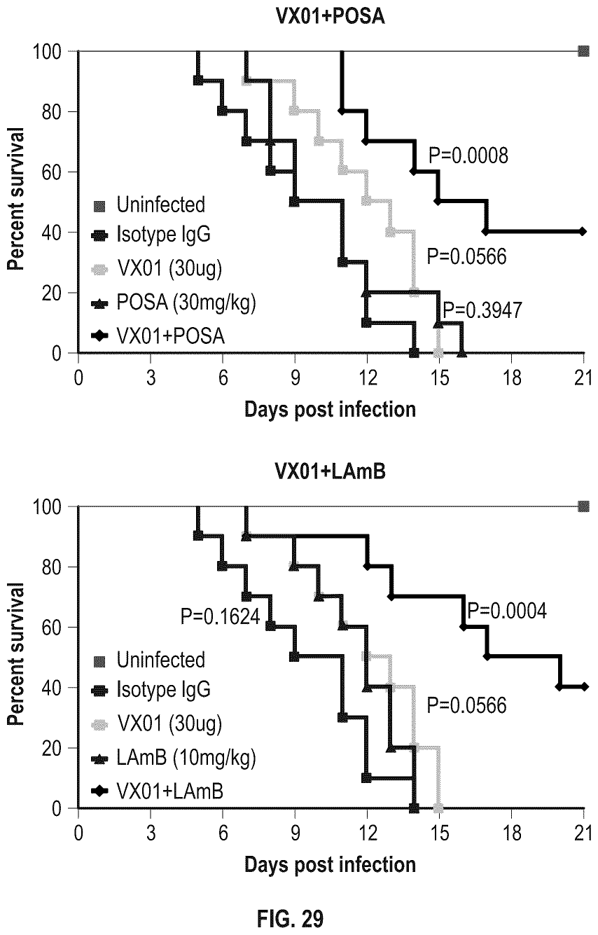

Finally, a steady state analysis was performed using the Gator system whereby a 1:2 serial dilution of CotH3 from 480 nM to 7.5 nM (specifically 480, 240, 120, 60, 30, 15, and 7.5 nM) compared to buffer alone for all three humanized variants and the chimera (FIGS. 8 and 9). In this type of analysis, the KD values were all very similar ranging from 4 to 6 nM, with the chimera and L3:H3 humanized variant exhibiting KD values of 5.2 and 5.4 nM, respectively, with high R2 values of 0.94 and 0.95, respectively.

### CONCLUSIONS

The original goal of this project was to humanize a murine mAb against CotH3 protein. Since the murine mAb had a reported affinity for the CotH3 antigen of ˜50 nM, which was confirmed by AvantGen, a second goal was to affinity mature the humanized antibody to improve the KD value by 10-fold. We noted that the first (6E11A8) and second sequencing (AvantGen) of the original mouse monoclonal antibody (C2) variable kappa chain region results had multiple differences. Both V kappa sequences were used to generate a chimeric form with the human IgG₁ constant region. Curiously, simply by generating a chimeric form with the murine VH/VK coupled to human constant regions, resulted in a marked improvement of affinity, with the chimeric form exhibiting a KD value of 5.7 nM for the 6E11A8 LC chimera (Chimera 1) and 1.8 nM for the AvantGen LC-chimera (Chimera 2), which represented the desired 10-fold improvement in binding affinity. For humanization, the best fit human acceptor sequences were 4-59*01 for the murine VH and A18 for the murine Vx. The grafted variant using these frameworks was used to generate the grafted humanized variant, which was produced in ExpiCHO cells. However, this variant exhibited no antigen binding activity. Computer modeling led to the production of 3 humanized LCs carrying one, two or 3 sets of backmutations and 3 humanized HCs also carrying one, two or three sets of back-mutations in the framework regions. The L3:H3 variant with a total of 6 back-mutations in the LC (S53K, R54L, F55D, Y34N and Y36L and L46R) and 4 back-mutations in the HC (S35N, W47H, N58F and V71R) exhibited a KD value for CotH3 within 2-fold of the chimera based on the association rate and Kobs and almost identical (5 nM) when assessed by steady state methodology. Therefore, the goals of the project to obtain a humanized variant with a KD value within 2-fold of that of the chimera and with an improvement of 10-fold above the parental murine mAb were met. We have also shown that the humanized LC3:HC3 variant binds antigen by Western blot, and was able to bind clinical fungal isolates (e.g. Cunninghamella and Mucor circinelloides), similar to the parental mAb as measured by flow cytometry. In functional in vitro assays, L3:H3 enhances neutrophil killing of R. delemar 99-880. Further, in in vivo assays using neutropenic mice infected by R. delemar 99-880 or Mucor circinelloides, L3:H3 showed similar efficacy in reducing mortality as the parental mouse monoclonal antibody.

### Materials and Methods

Western blotting: The Flag tag-CotH3 construct was created using the yeast plasmid pXW55-URA3. To generate the pXW55-FLAG-TEV-CotH3-URA3 [8063-base pair (bp)] construct, 1705-bp CotH3 complementary DNA (cDNA) was synthesized from R. delemar 99-880 using an RNA extraction kit (Qiagen, Hilden, Germany) and a cDNA synthesis kit (Promega, Madison, WI) according to the manufacturers' instructions. The synthesized CotH3 cDNA was flanked by Flag-TEV sequences at the 5′ end using polymerase chain reaction (PCR) and then subcloned into the Spe I and Pml I cloning site. The constructed vector was transformed into S. cerevisiae BJ5464, and the positive colonies were selected on uracil-free YNB medium. The positive yeast colonies containing the right constructs appeared after incubation for 3 days at 30° C., which were then confirmed by PCR and sequencing.

To produce the rCotH3p, a clone was grown in YPD medium at 30° C. for 3 days with shaking at 200 rpm. The cells were centrifuged and the pellet was washed with tris-buffered saline (TBS) before suspending in a small volume of cold TBS. Suspended yeast cells were then disrupted by sonication for 30 min with 1-min intervals interrupted with 1-min storing on ice. The lysate was then centrifuged at 10,000 g for 30 min. The collected supernatant was filtered through a 0.20-μm filter, followed by purification using anti-Flag magnetic beads (Sigma-Aldrich) according to the manufacturer's instructions. The purity of CotH3 protein was measured by SDS-PAGE gel. Endotoxin level was measured by LAL as above. The use of yeast as an expression system provided endotoxin-free protein.

For Western blotting, 0.1 μg Flag-CotH3 or 10 μg of each sample total lysate was used to separate proteins on an SDS-PAGE. Separated proteins were transferred to PVDF membranes (GE Water & Process Technologies) and treated with Western blocking reagent (Roche) for overnight at 4° C. IgG anti-CotH3-LC2HC3 (0.02 μg/ml), The IgG anti-CotH3-LC3HC2 (0.02 g/ml), and IgG anti-CotH3-LC3HC3 (0.02 μg/ml), were used as primary antibodies. After 1 h, 0.2 μg/ml of HRP-IgG anti-human IgG (Invitrogen, Cat #31412) secondary antibody was added for another 1 h at room temperature. Flag-CotH3 bands were visualized by adding the HRP substrate (SuperSignal West Dura Extended Duration Substrate, Thermo Scientific), and the chemiluminescent signal was detected using an In-gel Azure Imager c400 fluorescence system (Azure Biosystems).

### Staining of Fungal Cell for Surface Antigen for Acquisition in Flow Cytometry:

Spores were collected in endotoxin-free Dulbecco's phosphate-buffered saline (PBS) containing 0.01% Tween 80 for Mucorales. Collected spores were washed with PBS and counted with a hemocytometer. Count the cells and centrifuge to pellet the cells. Re-suspend cells in 2 ml 1×PBS and add 2 ml of 4% paraformaldehyde. Incubate for 30 minutes at 4 degree. The fixed cells were washed three times with PBS, 2×10⁶ cells per tube were used for staining. The cells were centrifuged to pellet, cell pellet were blocked by 2% BSA in PBS in 100 μl per tube. IgG anti-CotH3-LC2HC3 (20 μg/ml), IgG anti-CotH3-LC3HC2 (20 μg/ml), and IgG anti-CotH3-LC3HC3 (20 μg/ml), were used as primary antibodies in blocking buffer. After 1 h, 1 μg/ml of AlexaFluor 488-IgG anti-mouse IgG (Invitrogen, Cat #A11013) secondary antibody in blocking buffer was added for another 1 h at room temperature. 20,000 total events was Acquired by flow cytometry.

PMN phagocytosis assay: R. delemar spores were collected and washed twice with HBSS and counted. The spores count been adjusted at 1×10⁵ spores/ml using 1×RPMI 1640 media (10% FBS supplemented, 1% Pencillin/Strepomycin). 100 l of spores from above stock added to each well. IgG anti-CotH3-LC2HC3, IgG anti-CotH3-LC3HC2, IgG anti-CotH3-LC3HC3, or isotype antibody were prepared at 100 μg/ml as the stock in 1×RPMI 1640 media (10% FBS supplemented, 1% Pencillin/Strepomycin). 3 μl of the humanized antibody or the isotype antibody were added to their respective test well from each stock. Spores with antibodies were incubated for 30 min at Room Temperature before neutrophil treatment. Neutrophil cell were isolated from human blood and been adjust at 1×10⁵ cells/ml or 2×10⁵ cells/ml by using 1×RPMI 1640 media (10% FBS supplemented, 1% Penicillin/Streptomycin). 100 μl of the neutrophils were added to each well designated for killing activity leaving wells for spore only control. The plate was incubated at 37° C. for 2 hours for killing, following by 4° C. overnight treatment in cold-room to kill all the neutrophils. The remaining lived spores from each experiment group and the control group spores were plate on PDA+0.1% Triton plate with serial dilutions. The Rhizopus colonies were counted the next day.

Mouse models: Neutropenic mice model used male ICR mice (20 to 23 g) obtained from Envigo (NJ). Mice were injected with cyclophosphamide (200 mg/kg, given intraperitoneally) and cortisone acetate (500 mg/kg, given subcutaneously) on days −2, +3, and +8 relative to infection⁽¹⁾.

Infection and treatment: Neutropenic mice were infected intratracheally with 25 μl of PBS containing 2.5×10⁵ spores of Rhizopus delemear, or 2.5×10⁶ of M. circinelloides, respectively⁽²⁾. Immediately after challenge, three mice from each group infected with different Mucorales were euthanized and their lungs were homogenized in PBS and quantitatively cultured on PDA plates containing 0.1% Triton X-100. Colonies were counted after 24-hour incubation period at 37° C. After infection, mice were treated daily with 5 mg in 0.2 ml of irrigation water with ceftazidime subcutaneously to prevent bacterial superinfection. Antibody treatment with a single dose of 30 μg humanized antibody, or isotype human IgG started 24 or 48 hours after infection and was administered by intraperitoneal injection. Time to moribundity (equated with survival) served as the primary endpoint. As a secondary endpoint and in some experiments, fungal burden in the lungs and brains (primary and secondary target organs) was determined 96 hours after infection by quantitative PCR assay, as we previously described⁽³⁾. Values were expressed as log₁₀ spore equivalent per gram of tissue. Histopathological examination was carried out on sections of the harvested organs after fixing in 10% zinc formalin. The fixed organs were embedded in paraffin, and 5-mm sections were stained with hematoxylin and eosin.

For determining the protective activity of the monoclonal antibodies with antifungal drugs, neutropenic mice were infected with Rhizopus delemar or M. circinelloides and treated with the humanized antibody, isotype IgG, posaconazole (Merck and Co., Kenilworth, NJ), LAmB (Gilead Sciences, Foster City, CA), or a combination of the humanized antibody and either antifungal drugs started 48 hours after infection. Humanized antibody was given once by intraperitoneal injection at 30 μg, posaconazole was administered by oral gavage at 30 mg/kg twice daily for 7 days, and LAmB was given intravenously at 10 mg/kg per day for 4 days. Time to moribund state and tissue fungal burden served as end points as above.

Pharmacokinetics study: Neutropenic mice were infected intratracheally with 25 μl of PBS containing 2.5×10⁵ spores of Rhizopus delemear. Immediately after challenge, three mice from each group infected with different Mucorales were euthanized and their lungs were homogenized in PBS and quantitatively cultured on PDA plates containing 0.1% Triton X-100. Colonies were counted after 24-hour incubation period at 37° C. After infection, mice were treated daily with 5 mg in 0.2 ml of irrigation water with ceftazidime subcutaneously to prevent bacterial superinfection. Humanized IgG LC3HC3 or Mouse IgG C2 antibody treatment with a single dose of 10 μg, 30 μg, or 100 μg started 24 after infection and was administered by intravenous injection. Mouse serum samples were collected at the time points: 1 hour, 2 hours, 4 hours, 8 hours, 24 hours, 72 hours, 120 hours, and 168 hours after the antibody injection. The antibody concentration in the mouse serum samples was detected by ELISA, with Flag-CotH3 as the coating protein, and anti-human-IgG-HRP as the secondary antibody.

### REFERENCES

1. Luo G, Gebremariam T, Lee H, French S W, Wiederhold N P, Patterson T F, et al. Efficacy of liposomal amphotericin B and posaconazole in intratracheal models of murine mucormycosis. Antimicrob Agents Chemother. 2013; 57(7):3340-7.
2. Gebremariam T, Alkhazraji S, Soliman S S M, Gu Y, Jeon H H, Zhang L, et al. Anti-CotH3 antibodies protect mice from mucormycosis by prevention of invasion and augmenting opsonophagocytosis. Sci Adv. 2019; 5(6):eaaw1327.
3. Ibrahim A S, Bowman J C, Avanessian V, Brown K, Spellberg B, Edwards J E, Jr., et al. Caspofungin inhibits Rhizopus oryzae 1,3-beta-D-glucan synthase, lowers burden in brain measured by quantitative PCR, and improves survival at a low but not a high dose during murine disseminated zygomycosis. Antimicrob Agents Chemother. 2005; 49(2):721-7.

### Example 2—Additional Embodiments of Antibody Sequences

Heavy Chain Variable Region 6E11A8 (nucleic acid sequence)
SEQ ID NO: 41:
CAGCTTCAGGAGTCAGGACCTAGCCTCGTGAAACCTTCTCAGACTCTGTCCCTCACC
TGTTCTGTCACTGGCGACTCCATCACCAGTGGTTACTGGAACTGGATCCGGAAATTC
CCAGGGAATAAACTTGAACACATGGGGTACATAAAGTACAGTGGTCGCACTTTCTA
CAATCCATCTCTCAAAAGTCGAGTCTCCATCACTCGAGACACATCCAAGAACCAGTA
CTACCTGCAGTTGAATTCTGTGACTTCTGAGGACACAGCCACATATTATTGTGCAAG
CCGCGGCTACTGGGGCCAAGGCACCACTCTCACAGTCTCCTCA
Light Chain Variable Region 6E11A8 (nucleic acid sequence)
SEQ ID NO: 42:
GATGTTGTGATGACCCAGACTCCACTCACTTTGTCGGTTACCATTGGACAACCAGCC
TCCATCTCTTGCAAGTCAAGTCAGAGCCTCTTAGATAGTGACGGAAAGACATTTTTG
AATTGGTTGTTACAGAGGCCAGGCCAGTCTCCAAAGCGCCTAATCTATCTGGTGTCT
AAACTGGACTCTGGAGTCCCTGACAGGTTCACTGGCACTGGATCAGGGACAGATTTC
ACACTGAAAATCAGCAGAGTGGAGGCTGAGGATTTGGGAGTTTATTATTGCTGGCA
AGGTACACATTTTCCTCACACGTTCGGTGCTGGGACCAAGCTGGAGCTGAAACGG
Humanized Variant LC1 (nucleic acid sequence)
SEQ ID NO: 43:
ATGGAAACCGGGCTGAGATGGCTGCTGCTGGTCGCTGTGCTTAAGGGAGTCCAGTGT
GACATCGTGATGACCCAGACACCTCTGAGCCTGAGCGTGACACCTGGACAGCCTGC
CAGCATCAGCTGCAAGTCTAGCCAGAGCCTGCTGGACTCCGACGGCAAGACCTTCCT
GTACTGGTATCTGCAGAAGCCCGGCCAGTCTCCTCAGCTGCTGATCTACCTGGTGTC
CAAGCTGGATAGCGGCGTGCCCGATAGATTTTCTGGCTCTGGCAGCGGCACCGACTT
CACCCTGAAGATCTCTAGAGTGGAAGCCGAGGACGTGGGCGTGTACTACTGTTGGC
AGGGCACACACTTCCCTCACACCTTTGGACAGGGTACCAAGGTCGAGATCAAACGC
ACAGTGGCCGCTCCATCTGTCTTCATTTTTCCACCCAGTGACGAACAGCTGAAAAGC
GGGACTGCCTCCGTGGTCTGTCTGCTGAACAATTTCTACCCCCGGGAAGCCAAGGTG
CAGTGGAAAGTCGATAACGCTCTGCAGTCAGGCAATAGCCAGGAGTCCGTGACCGA
ACAGGACTCTAAGGATAGTACATATTCACTGAGTTCAACTCTGACCCTGTCCAAAGC
AGACTACGAGAAGCATAAAGTGTATGCCTGTGAAGTCACCCACCAGGGGCTGTCCT
CACCAGTCACTAAGTCCTTCAATAGGGGCGAATGCTGA
Humanized Variant HC1 (nucleic acid sequence)
SEQ ID NO: 44:
ATGGATACAAGGGCTCCTACTCAGCTGCTGGGCCTGCTGCTGCTGTGGCTGCCTGGA
TCCAGATGTCAAGTGCAGCTCCAAGAGTCTGGCCCTGGCCTGGTCAAGCCTAGCGA
AACACTGAGCCTGACCTGTACCGTGTCCGGCGATAGCATCACCAGCGGCTACTGGTC
CTGGATCAGACAGCCTCCTGGCAAAGGCCTGGAATGGATCGGCTACATCAAGTACA
GCGGCCGGACCAACTACAACCCCAGCCTGAAGTCCAGAGTGACCATCAGCCGGGAC
ACCAGCAAGAACCAGTTCTCCCTGAAGCTGAGCAGCGTGACAGCCGCCGATACAGC
CGTGTACTACTGTGCCAGCAGAGGCTATTGGGGCCAGGGCACACTGGTCACAGTGTC
TAGCGCCAGCACAAAGGGGCCCTCTGTGTTCCCTCTGGCCCCCAGCAGCAAGAGCA
CATCTGGCGGAACAGCCGCCCTGGGCTGCCTGGTGAAAGACTACTTCCCCGAGCCC
GTGACCGTGTCCTGGAACTCTGGCGCCCTGACCAGCGGCGTGCACACCTTTCCAGCC
GTGCTGCAGAGCAGCGGCCTGTACAGCCTGAGCAGCGTGGTGACAGTGCCCAGCAG
CAGCCTGGGCACCCAGACCTACATCTGCAACGTGAACCACAAGCCCAGCAACACCA
AGGTGGACAAGAAGGTGGAACCCAAGAGCTGCGACAAGACCCACACCTGTCCCCCC
TGCCCTGCCCCTGAACTGCTGGGCGGACCCAGCGTGTTCCTGTTCCCCCCAAAGCCC
AAGGACACCCTGATGATCAGCCGGACCCCCGAAGTGACCTGCGTGGTGGTGGACGT
GTCCCACGAGGACCCTGAAGTGAAGTTCAATTGGTACGTGGACGGCGTGGAAGTGC
ACAACGCCAAGACCAAGCCCAGAGAGGAACAGTACAACAGCACCTACCGGGTGGT
GTCCGTGCTGACCGTGCTGCACCAGGACTGGCTGAACGGCAAAGAGTACAAGTGCA
AGGTGTCCAACAAGGCCCTGCCTGCCCCCATCGAGAAAACCATCAGCAAGGCCAAG
GGCCAGCCCCGCGAGCCCCAGGTGTACACACTGCCCCCCAGCCGGGACGAGCTGAC
CAAGAACCAGGTGTCCCTGACCTGTCTGGTGAAAGGCTTCTACCCCAGCGATATCGC
CGTGGAATGGGAGAGCAACGGCCAGCCCGAGAACAACTACAAGACCACCCCCCCTG
TGCTGGACAGCGACGGCTCATTCTTCCTGTACAGCAAGCTGACCGTGGACAAGAGC
CGGTGGCAGCAGGGCAACGTGTTCAGCTGCAGCGTGATGCACGAGGCCCTGCACAA
CCACTACACCCAGAAGTCCCTGAGCCTGAGCCCCGGCAAGTGA
Humanized Variant LC2 (nucleic acid sequence)
SEQ ID NO: 45:
ATGGAAACCGGGCTGAGATGGCTGCTGCTGGTCGCTGTGCTTAAGGGAGTCCAGTGT
GACATCGTGATGACCCAGACACCTCTGAGCCTGAGCGTGACACCTGGACAGCCTGC
CAGCATCAGCTGCAAGTCTAGCCAGAGCCTGCTGGACTCCGACGGCAAGACCTTCCT
GAACTGGCTGCTGCAGAAGCCCGGCCAGTCTCCTCAGCTGCTGATCTACCTGGTGTC
CAAGCTGGATAGCGGCGTGCCCGATAGATTTTCTGGCTCTGGCAGCGGCACCGACTT
CACCCTGAAGATCTCTAGAGTGGAAGCCGAGGACGTGGGCGTGTACTACTGTTGGC
AGGGCACACACTTCCCTCACACCTTTGGACAGGGTACCAAGGTCGAGATCAAACGC
ACAGTGGCCGCTCCATCTGTCTTCATTTTTCCACCCAGTGACGAACAGCTGAAAAGC
GGGACTGCCTCCGTGGTCTGTCTGCTGAACAATTTCTACCCCCGGGAAGCCAAGGTG
CAGTGGAAAGTCGATAACGCTCTGCAGTCAGGCAATAGCCAGGAGTCCGTGACCGA
ACAGGACTCTAAGGATAGTACATATTCACTGAGTTCAACTCTGACCCTGTCCAAAGC
AGACTACGAGAAGCATAAAGTGTATGCCTGTGAAGTCACCCACCAGGGGCTGTCCT
CACCAGTCACTAAGTCCTTCAATAGGGGCGAATGCTGA
Humanized Variant HC2 (nucleic acid sequence)
SEQ ID NO: 46:
ATGGATACAAGGGCTCCTACTCAGCTGCTGGGCCTGCTGCTGCTGTGGCTGCCTGGA
TCCAGATGTCAAGTGCAGCTCCAAGAGTCTGGCCCTGGCCTGGTCAAGCCTAGCGA
AACACTGAGCCTGACCTGTACCGTGTCCGGCGATAGCATCACCAGCGGCTACTGGTC
CTGGATCAGACAGCCTCCTGGCAAAGGCCTGGAACACATCGGCTACATCAAGTACA
GCGGCCGGACCTTTTACAACCCCAGCCTGAAGTCCAGAGTGACCATCAGCCGGGAC
ACCAGCAAGAACCAGTTCTCCCTGAAGCTGAGCAGCGTGACAGCCGCCGATACAGC
CGTGTACTACTGTGCCAGCAGAGGCTATTGGGGCCAGGGCACACTGGTCACAGTGTC
TAGCGCCAGCACAAAGGGGCCCTCTGTGTTCCCTCTGGCCCCCAGCAGCAAGAGCA
CATCTGGCGGAACAGCCGCCCTGGGCTGCCTGGTGAAAGACTACTTCCCCGAGCCC
GTGACCGTGTCCTGGAACTCTGGCGCCCTGACCAGCGGCGTGCACACCTTTCCAGCC
GTGCTGCAGAGCAGCGGCCTGTACAGCCTGAGCAGCGTGGTGACAGTGCCCAGCAG
CAGCCTGGGCACCCAGACCTACATCTGCAACGTGAACCACAAGCCCAGCAACACCA
AGGTGGACAAGAAGGTGGAACCCAAGAGCTGCGACAAGACCCACACCTGTCCCCCC
TGCCCTGCCCCTGAACTGCTGGGCGGACCCAGCGTGTTCCTGTTCCCCCCAAAGCCC
AAGGACACCCTGATGATCAGCCGGACCCCCGAAGTGACCTGCGTGGTGGTGGACGT
GTCCCACGAGGACCCTGAAGTGAAGTTCAATTGGTACGTGGACGGCGTGGAAGTGC
ACAACGCCAAGACCAAGCCCAGAGAGGAACAGTACAACAGCACCTACCGGGTGGT
GTCCGTGCTGACCGTGCTGCACCAGGACTGGCTGAACGGCAAAGAGTACAAGTGCA
AGGTGTCCAACAAGGCCCTGCCTGCCCCCATCGAGAAAACCATCAGCAAGGCCAAG
GGCCAGCCCCGCGAGCCCCAGGTGTACACACTGCCCCCCAGCCGGGACGAGCTGAC
CAAGAACCAGGTGTCCCTGACCTGTCTGGTGAAAGGCTTCTACCCCAGCGATATCGC
CGTGGAATGGGAGAGCAACGGCCAGCCCGAGAACAACTACAAGACCACCCCCCCTG
TGCTGGACAGCGACGGCTCATTCTTCCTGTACAGCAAGCTGACCGTGGACAAGAGC
CGGTGGCAGCAGGGCAACGTGTTCAGCTGCAGCGTGATGCACGAGGCCCTGCACAA
CCACTACACCCAGAAGTCCCTGAGCCTGAGCCCCGGCAAGTGA
Humanized Variant LC3 (nucleic acid sequence)
SEQ ID NO: 47:
ATGGAAACCGGGCTGAGATGGCTGCTGCTGGTCGCTGTGCTTAAGGGAGTCCAGTGT
GACATCGTGATGACCCAGACACCTCTGAGCCTGAGCGTGACACCTGGACAGCCTGC
CAGCATCAGCTGCAAGTCTAGCCAGAGCCTGCTGGACTCCGACGGCAAGACCTTCCT
GAACTGGCTGCTGCAGAAGCCCGGCCAGTCTCCTCAGCGGCTGATCTACCTGGTGTC
CAAGCTGGATAGCGGCGTGCCCGATAGATTTTCTGGCTCTGGCAGCGGCACCGACTT
CACCCTGAAGATCTCTAGAGTGGAAGCCGAGGACGTGGGCGTGTACTACTGTTGGC
AGGGCACACACTTCCCTCACACCTTTGGACAGGGTACCAAGGTCGAGATCAAACGC
ACAGTGGCCGCTCCATCTGTCTTCATTTTTCCACCCAGTGACGAACAGCTGAAAAGC
GGGACTGCCTCCGTGGTCTGTCTGCTGAACAATTTCTACCCCCGGGAAGCCAAGGTG
CAGTGGAAAGTCGATAACGCTCTGCAGTCAGGCAATAGCCAGGAGTCCGTGACCGA
ACAGGACTCTAAGGATAGTACATATTCACTGAGTTCAACTCTGACCCTGTCCAAAGC
AGACTACGAGAAGCATAAAGTGTATGCCTGTGAAGTCACCCACCAGGGGCTGTCCT
CACCAGTCACTAAGTCCTTCAATAGGGGCGAATGCTGA
Humanized Variant HC3 (nucleic acid sequence)
SEQ ID NO: 48:
ATGGATACAAGGGCTCCTACTCAGCTGCTGGGCCTGCTGCTGCTGTGGCTGCCTGGA
TCCAGATGTCAAGTGCAGCTCCAAGAGTCTGGCCCTGGCCTGGTCAAGCCTAGCGA
AACACTGAGCCTGACCTGTACCGTGTCCGGCGATAGCATCACCAGCGGCTACTGGA
ATTGGATCAGACAGCCTCCTGGCAAAGGCCTGGAACACATCGGCTACATCAAGTAC
AGCGGCCGGACCTTTTACAACCCCAGCCTGAAGTCCAGAGTGACCATCAGCCGGGA
CACCAGCAAGAACCAGTTCTCCCTGAAGCTGAGCAGCGTGACAGCCGCCGATACAG
CCGTGTACTACTGTGCCAGCAGAGGCTATTGGGGCCAGGGCACACTGGTCACAGTGT
CTAGCGCCAGCACAAAGGGGCCCTCTGTGTTCCCTCTGGCCCCCAGCAGCAAGAGC
ACATCTGGCGGAACAGCCGCCCTGGGCTGCCTGGTGAAAGACTACTTCCCCGAGCC
CGTGACCGTGTCCTGGAACTCTGGCGCCCTGACCAGCGGCGTGCACACCTTTCCAGC
CGTGCTGCAGAGCAGCGGCCTGTACAGCCTGAGCAGCGTGGTGACAGTGCCCAGCA
GCAGCCTGGGCACCCAGACCTACATCTGCAACGTGAACCACAAGCCCAGCAACACC
AAGGTGGACAAGAAGGTGGAACCCAAGAGCTGCGACAAGACCCACACCTGTCCCCC
CTGCCCTGCCCCTGAACTGCTGGGCGGACCCAGCGTGTTCCTGTTCCCCCCAAAGCC
CAAGGACACCCTGATGATCAGCCGGACCCCCGAAGTGACCTGCGTGGTGGTGGACG
TGTCCCACGAGGACCCTGAAGTGAAGTTCAATTGGTACGTGGACGGCGTGGAAGTG
CACAACGCCAAGACCAAGCCCAGAGAGGAACAGTACAACAGCACCTACCGGGTGG
TGTCCGTGCTGACCGTGCTGCACCAGGACTGGCTGAACGGCAAAGAGTACAAGTGC
AAGGTGTCCAACAAGGCCCTGCCTGCCCCCATCGAGAAAACCATCAGCAAGGCCAA
GGGCCAGCCCCGCGAGCCCCAGGTGTACACACTGCCCCCCAGCCGGGACGAGCTGA
CCAAGAACCAGGTGTCCCTGACCTGTCTGGTGAAAGGCTTCTACCCCAGCGATATCG
CCGTGGAATGGGAGAGCAACGGCCAGCCCGAGAACAACTACAAGACCACCCCCCCT
GTGCTGGACAGCGACGGCTCATTCTTCCTGTACAGCAAGCTGACCGTGGACAAGAG
CCGGTGGCAGCAGGGCAACGTGTTCAGCTGCAGCGTGATGCACGAGGCCCTGCACA
ACCACTACACCCAGAAGTCCCTGAGCCTGAGCCCCGGCAAGTGA
CotH3 (RO3G_11882) SEQ ID NO: 35
MKLSIISAAFLVAITHAASIKENVIAPNATDVKVSVNGQQVTLTASDANVPYFTGSAEVG
ASKTYKYVAGGTEESFDRSLDGITNSTLNDFYNRPVTYANLPQLPWPIEKDPQWTRSGS
KADIFDDNYIPSVFFHGDDSQVQNVVKNVPADRISGTLTFIGSNYVYSFQNVSFGIHGAG
KKHNNAKQSWNWILSGSDTMGNRNFFKLRHMEEDPTQIRERLYSDILHAMGTYANDA
TMVRLFINNQGFGTFNMLDDITQFSYINAKFYNGKPPATLGPLYDGASGADFLYHPGNL
DGYSSWVANTANPNGEAYEALDPLCKAWNETTYTDNTAIANFEKMFDLDRFMRFMVI
EYLTADWDGYWMGQTNDGAYRDPTDNNKWYFLDQDFDGTFGVNLAAPEGNAFLDVS
YKDFPSRYPGAVMINNLLQNADKKATFEKYLTETVRVLFNNVTLTNRVLALHNFLLPDL
EWDRSIVQQSPGINFGWTFDQVTQNLWQGVTAPNNNGGGAAFGLVEYIAAKAQAVAK
EFNISIVSQPVGPPSANGTTAAAPAPAAGNSTGKGGNQSISSSASSNKTSAQSTSGASRSK
TAPIVLAISALALLVF*
CotH3 from Rhizopus oryzae 99-880 (predicted amino acid)
SEQ ID NO: 36
MKLSIISAAFLVAITHAASIKFNVIAPNATDVKVSVNGQQVTLTASDANVPYFTGSAEVG
ASKTYKYVAGGTEESFDRSLDGITNSTLNDFYNRPVTYANLPQLPWPIEKDPQWTRSGS
KADIFDDNYIPSVFFHGDDSQVQNVVKNVPADRISGTLTFIGSNYVYSFQNVSFGIHGAG
KKHNNAKQSWNWILSGSDTMGNRNFFKLRHMEEDPTQIRERLYSDILHAMGTYANDA
TMVRLFINNQGFGTFNMLDDITQFSYINAKFYNGKPPATLGPLYDGASGADFLYHPGNL
DGYSSWVANTANPNGEAYEALDPLCKAWNETTYTDNTAIANFEKMFDLDRFMRFMVI
EYLTADWDGYWMGQTNDGAYRDPTDNNKWYFLDQDFDGTFGVNLAAPEGNAFLDVS
YKDFPSRYPGAVMINNLLQNADKKATFEKYLTETVRVLFNNVTLTNRVLALHNFLLPDL
EWDRSIVQQSPGINFGWTFDQVTQNLWQGVTAPNNNGGGAAFGLVEYIAAKAQAVAK
EFNISIVSQPVGPPSANGTTAAAPAPAAGNSTGKGGNQSISSSASSNKTSAQSTSGASRSK
TAPIVLAISALALLVF
CotH3 from Lichtheimia corymbifera-SEQ ID NO: 37
MKLSIISAAFLVAITHAASIKFNVIAPNATDVKVSVNGQQVTLTASDANVPYFTGSAEVG
SSKTYKYVAGGTEESFDRSLDGITNSTLNDFYNRPVTYANLPQLPWPIEKDPQWTRSGN
KADIFDDNYIPSVFFHGDDSQVQNVVKNVPADRISGTLTFIGSNYVYSFQNVSFGIHGAG
KKHNNAKQSWNWILSGSDTMGNRNFFKLRHMEEDPTQIRERLYSDILHAMGTYANDA
TMVRLFINNQGFGTFNMLDDITQFSYINAKFYNGKPPATLGPLYDGASGADFLYHPGNL
DGYSSWVANTANPNGEAYEALDPLCKAWNETTYTDNTAIANFEKMFDLDRFMRFMVI
EYLTADWDGYWMGQTNDGAYRDPTDNNKWYFLDQDFDGTFGVNLAAPEGNAFLDVS
YKDFPSRYPGAVMINNLLQNADKKATYEKYLTETVRVLFNNVTLTNRVLALHNFLLPD
LEWDRSIVQQSPGINFGWTFDQVTQNLWQGVTAPNNNGGGAAFGLVEYIATKAQAVA
KEFNISIVSQPVGPPSANGTTAAAPAPAAGNSTGKGGNQSISSSASSNKTSAQSTSGASRS
KTAPIIFSHFRFSSPLYSK

The entirety of each patent, patent application, publication or any other reference or document cited herein hereby is incorporated by reference. In case of conflict, the specification, including definitions, will control.

Citation of any patent, patent application, publication or any other document is not an admission that any of the foregoing is pertinent prior art, nor does it constitute any admission as to the contents or date of these publications or documents.

Unless otherwise defined, all technical and scientific terms used herein have the same meaning as commonly understood by one of ordinary skill in the art to which this invention belongs. Although methods and materials similar or equivalent to those described herein can be used in the practice or testing of the present invention, suitable methods and materials are described herein.

All of the features disclosed herein may be combined in any combination. Each feature disclosed in the specification may be replaced by an alternative feature serving a same, equivalent, or similar purpose. Thus, unless expressly stated otherwise, disclosed features (e.g., antibodies) are an example of a genus of equivalent or similar features.

As used herein, all numerical values or numerical ranges include integers within such ranges and fractions of the values or the integers within ranges unless the context clearly indicates otherwise. Further, when a listing of values is described herein (e.g., about 50%, 60%, 70%, 80%, 85% or 86%) the listing includes all intermediate and fractional values thereof (e.g., 54%, 85.4%). Thus, to illustrate, reference to 80% or more identity, includes 81%, 82%, 83%, 84%, 85%, 86%, 87%, 88%, 89%, 90%, 91%, 92%, 93%, 94% etc., as well as 81.1%, 81.2%, 81.3%, 81.4%, 81.5%, etc., 82.1%, 82.2%, 82.3%, 82.4%, 82.5%, etc., and so forth.

Reference to an integer with more (greater) or less than includes any number greater or less than the reference number, respectively. Thus, for example, a reference to less than 100, includes 99, 98, 97, etc. all the way down to the number one (1); and less than 10, includes 9, 8, 7, etc. all the way down to the number one (1).

As used herein, all numerical values or ranges include fractions of the values and integers within such ranges and fractions of the integers within such ranges unless the context clearly indicates otherwise. Thus, to illustrate, reference to a numerical range, such as 1-10 includes 1, 2, 3, 4, 5, 6, 7, 8, 9, 10, as well as 1.1, 1.2, 1.3, 1.4, 1.5, etc., and so forth. Reference to a range of 1-50 therefore includes 1, 2, 3, 4, 5, 6, 7, 8, 9, 10, 11, 12, 13, 14, 15, 16, 17, 18, 19, 20, etc., up to and including 50, as well as 1.1, 1.2, 1.3, 1.4, 1.5, etc., 2.1, 2.2, 2.3, 2.4, 2.5, etc., and so forth.

Reference to a series of ranges includes ranges which combine the values of the boundaries of different ranges within the series. Thus, to illustrate reference to a series of ranges, for example, of 1-10, 10-20, 20-30, 30-40, 40-50, 50-60, 60-75, 75-100, 100-150, 150-200, 200-250, 250-300, 300-400, 400-500, 500-750, 750-1,000, 1,000-1,500, 1,500-2,000, 2,000-2,500, 2,500-3,000, 3,000-3,500, 3,500-4,000, 4,000-4,500, 4,500-5,000, 5,500-6,000, 6,000-7,000, 7,000-8,000, or 8,000-9,000, includes ranges of 10-50, 50-100, 100-1,000, 1,000-3,000, 2,000-4,000, etc.

Modifications can be made to the foregoing without departing from the basic aspects of the technology. Although the technology has been described in substantial detail with reference to one or more specific embodiments, those of ordinary skill in the art will recognize that changes can be made to the embodiments specifically disclosed in this application, yet these modifications and improvements are within the scope and spirit of the technology.

The invention is generally disclosed herein using affirmative language to describe the numerous embodiments and aspects. The invention also specifically includes embodiments in which particular subject matter is excluded, in full or in part, such as substances or materials, method steps and conditions, protocols, or procedures. For example, in certain embodiments or aspects of the invention, materials and/or method steps are excluded. Thus, even though the invention is generally not expressed herein in terms of what the invention does not include aspects that are not expressly excluded in the invention are nevertheless disclosed herein.

The technology illustratively described herein suitably can be practiced in the absence of any element(s) not specifically disclosed herein. Thus, for example, in each instance herein any of the terms “comprising,” “consisting essentially of,” and “consisting of” can be replaced with either of the other two terms. The terms and expressions which have been employed are used as terms of description and not of limitation, and use of such terms and expressions do not exclude any equivalents of the features shown and described or segments thereof, and various modifications are possible within the scope of the technology claimed. The term “a” or “an” can refer to one of or a plurality of the elements it modifies (e.g., “a reagent” can mean one or more reagents) unless it is contextually clear either one of the elements or more than one of the elements is described. The term “about” as used herein refers to a value within 10% of the underlying parameter (i.e., plus or minus 10%), and use of the term “about” at the beginning of a string of values modifies each of the values (i.e., “about 1, 2 and 3” refers to about 1, about 2 and about 3). For example, a weight of “about 100 grams” can include weights between 90 grams and 110 grams. The term, “substantially” as used herein refers to a value modifier meaning “at least 95%”, “at least 96%”, “at least 97%”, “at least 98%”, or “at least 99%” and may include 100%. For example, a composition that is substantially free of X, may include less than 5%, less than 4%, less than 3%, less than 2%, or less than 1% of X, and/or X may be absent or undetectable in the composition.

Thus, it should be understood that although the present technology has been specifically disclosed by representative embodiments and optional features, modification and variation of the concepts herein disclosed can be resorted to by those skilled in the art, and such modifications and variations are considered within the scope of this technology.

What is claimed is:

1. A CotH3 binding agent comprising:
a light chain variable region comprising a CDR-L1, a CDR-L2 and a CDR-L3, wherein
(i) the CDR-L1 comprises an amino acid sequence at least 80% identical to an amino acid sequence of a CDR-L1 selected from SEQ ID Nos: 1-3,
(ii) the CDR-L2 comprises an amino acid sequence at least 80% identical to an amino acid sequence of a CDR-L2 selected from SEQ ID Nos: 4-5, and
(iii) the CDR-L3 comprises an amino acid sequence at least 80% identical to an amino acid sequence of a CDR-L3 selected from SEQ ID Nos: 6-7;
and a heavy chain variable region comprising a CDR-H1, a CDR-H2 and a CDR-H3, wherein
(iv) the CDR-H1 comprises an amino acid sequence at least 80% identical to the amino acid sequence set forth in SEQ ID NO:21,
(v) the CDR-H2 comprises an amino acid sequence at least 80% identical to the amino acid sequence set forth in SEQ ID NO:22, and
(vi) the CDR-H3 comprises an amino acid sequence at least 80% identical to the amino acid sequence set forth in SEQ ID NO:23;
wherein the CotH3 binding agent specifically binds to CotH3, or a portion thereof.

2. The CotH3 binding agent of claim 1, wherein,
(i) the CDR-L1 comprises the amino acid sequence set forth in SEQ ID NO:3,
(ii) the CDR-L2 comprises the amino acid sequence set forth in SEQ ID NO:4, and
(iii) the CDR-L3 comprises the amino acid sequence set forth in SEQ ID NO:6.

3. The CotH3 binding agent of claim 1, wherein,
(i) the CDR-L1 comprises the amino acid sequence set forth in SEQ ID NO:2,
(ii) the CDR-L2 comprises the amino acid sequence set forth in SEQ ID NO:5, and
(iii) the CDR-L3 comprises the amino acid sequence set forth in SEQ ID NO:7.

4. The CotH3 binding agent of any one of claim 1 to 3, wherein,
(i) the CDR-H1 comprises the amino acid sequence set forth in SEQ ID NO:21,
(ii) the CDR-H2 comprises the amino acid sequence set forth in SEQ ID NO:22, and
(iii) the CDR-H3 comprises the amino acid sequence set forth in SEQ ID NO:23.

5. The CotH3 binding agent of any one of claims 1 to 4, wherein the CotH3 binding agent comprises a variable light chain region at least 80% identical to a variable light chain region selected from Table 2 and a variable heavy chain regions at least 80% identical to a variable heavy chain region selected from Table 4.

6. The CotH3 binding agent of any one of claims 1 to 5, wherein the binding agent is a humanized, chimeric or CDR-grafted antibody.

7. The CotH3 binding agent of one of claims 1 to 6, wherein the CotH3 binding agent comprises a human heavy chain constant regions and/or a human light chain constant region.

8. The CotH3 binding agent of one of claims 1 to 7, wherein binding agent comprises an amino acid sequence of a light chain selected from a full length light chain region of Table 2 and an amino acid sequence of a heavy chain selected from a full length heavy chain of Table 4.

9. The CotH3 binding agent of any one of claims 1 to 8, wherein the CotH3 binding agent is a Fab, Fab′, single chain Fab (scFab), F(ab′)2, single chain Fab, Fv, Fd, single-chain Fv (scFv), disulfide-linked Fvs (sdFv), VL, VH, diabody ((VL-VH)2 or (VH-VL)2), triabody (trivalent), tetrabody (tetravalent), minibody ((scFV-CH3)2), IgGdeltaCH2, scFv-Fc or (scFv)2-Fc, or binding fragment thereof.

10. The CotH3 binding agent of claim 9, wherein the CotH3 binding agent is a single chain polypeptide.

11. The CotH3 binding agent of any one of claims 1 to 10, wherein the CotH3 binding agent is an antibody, or a binding fragment thereof.

12. The CotH3 binding agent of claim 11, wherein the CotH3 binding agent is a monoclonal antibody, or a binding fragment thereof.

13. The CotH3 binding agent of claim 11 or 12, wherein the CotH3 binding agent comprises a constant region of an IgG, IgD, IgE, IgA or IgM.

14. The CotH3 binding agent of claim 13, wherein the binding agent comprises a constant region selected from an IgG₁, IgG₂, IgG₃, and IgG₄.

15. The CotH3 binding agent of any one of claims 11 to 14, wherein the binding agent is a chimeric antibody comprising one or more human constant regions.

16. The CotH3 binding agent of any one of claims 1 to 15, wherein the binding agent comprises humanized or fully human framework regions.

17. The CotH3 binding agent of any one of claims 1 to 16, wherein the binding agent binds specifically to human CotH2.

18. The CotH3 binding agent of any one of claims 1 to 17, wherein the CotH3 binding agent binds to a Mucorales CotH3, a Mucorales CotH2, or a portion thereof, with a binding affinity (KD) of 50 nM or less.

19. The CotH3 binding agent of claim 18, wherein the CotH3 binding agent binds to a Mucorales CotH3, a Mucorales CotH2, or a portion thereof with a binding affinity (KD) of 10 nM or less.

20. The CotH3 binding agent of any one of claims 1 to 19, wherein the binding agent binds specifically to a protein comprising the amino acid sequence set forth in SEQ ID NO:34.

21. The CotH3 binding agent of any one of claims 1 to 20, wherein the binding agent binds specifically to the amino acid sequence set forth in SEQ ID NO:34 with a binding affinity (KD) of 50 nM or less, or with a binding affinity (KD) of 10 nM or less.

22. A pharmaceutical composition comprising the binding agent of any one of claims 1 to 21.

23. The pharmaceutical composition of claim 22, wherein the composition further comprises an anti-fungal agent.

24. The pharmaceutical composition of claim 23, wherein the anti-fungal agent is selected from the group consisting of amphotericin B, candicidin, filipin, hamycin, natamycin, nystatin, rimocidin, imidazoles, triazoles, thiazoles, allylamines, echinocandins, benzoic acid, ciclopirox, flucytosine, 5-fluorocytosine, griseofulvin, haloprogin, tolnaftate, undecylenic acid, crystal violet, Balsam of Peru, triterpenoids, and mixtures thereof.

25. The pharmaceutical composition of any one of claims 23 to 24, wherein the anti-fungal agent comprises a triazoles selected from albaconazole, efinaconazole, epoxiconazole, fluconazole, isavuconazole, itraconazole, posaconazole, propiconazole, ravuconazole, terconazole, voriconazole, and mixtures thereof.

26. The pharmaceutical composition of any one of claims 23 to 25, wherein the anti-fungal agent comprises posaconazole.

27. The pharmaceutical composition of claim 23, wherein the anti-fungal agent comprises LAmB (liposomal amphotericin B).

28. A method of preventing or treating a Mucorales infection in a subject comprising:
a) providing a subject having, suspected of having, or at risk of having a Mucorales infection; and
b) administering a therapeutically effective amount of a binding agent of any one of claims 1 to 21 to the subject, or administering a therapeutically effective amount of the pharmaceutical composition to the subject.

29. The method of claim 28, wherein the murcorales infection is caused by the presence of A. idahoensis, A. corymbifera, Apophysomyces elegans, Actinomucor elegans, A. rouxii, B. circina, B. multispora, C. brefeldii, C. angarensis, Cunninghamella bertholletiae (C. bertholletiae), Choanephora cucurbitarum, C. recurvatus, D. fulva, E. anomalus, H. elegans, H. assamensis, K. cordensis, Lichtheimia corymbifera (L. corymbifera), Lichtheimia ramosa, M. ambiguus, Mucor amphibiorum, Mucor circinelloides, M. verticillata, Parasitella parasitica, P. agaricine, P. anomala, P. circinans, Phycomyces blakesleeanus, S. umbellata, S. megalocarpus, T. elegans, T. indicae-seudaticae, Z. californiensis, Rhizomucor endophyticus, Rhizopus javensis, R. azygosporus, Rhizopus caespitosus, Rhizopus homothallicus, Rhizopus oryzae, R. delemar, Rhizopus stolonifer, Rhizopus reflexus, Rhizopus microsporus (e.g., var. rhizopodiformis), or Rhizopus schipperae.

30. The method of claim 28, wherein the Mucorales infection is caused by the presence of a Mucorales species of the genus Rhizopus.

31. The method of claim 30, wherein the Mucorales infection is caused by the presence of Rhizopus oryzae or R. delemar.

32. The method of any one of claims 28 to 31, wherein the subject is a human.

33. The method of any one of claims 28 to 31, wherein the binding agent specifically binds to at least 5 contiguous amino acids of a CotH3 polypeptide.

34. The method of any one of claims 28 to 33, wherein the binding agent specifically binds to polypeptide comprising the amino acid sequence of SEQ ID NO:34.

35. The method of any one of claims 28 to 33, wherein the binding agent is a monoclonal antibody or antigen binding fragment thereof.

36. The method of claim 35, wherein the antibody is a human chimeric antibody, a humanized antibody, a CDR grafted antibody or a combination thereof.

37. The method of claim 28, wherein the pharmaceutical composition further comprises an anti-fungal agent.

38. The method of claim 37, wherein the anti-fungal agent is selected from the group consisting of amphotericin B, candicidin, filipin, hamycin, natamycin, nystatin, rimocidin, imidazoles, triazoles, thiazoles, allylamines, echinocandins, benzoic acid, ciclopirox, flucytosine, 5-fluorocytosine, griseofulvin, haloprogin, tolnaftate, undecylenic acid, crystal violet, Balsam of Peru, triterpenoids, and mixtures thereof.

39. The method of any one of claims 37 to 38, wherein the anti-fungal agent comprises a triazoles selected from albaconazole, efinaconazole, epoxiconazole, fluconazole, isavuconazole, itraconazole, posaconazole, propiconazole, ravuconazole, terconazole, voriconazole, and mixtures thereof.

40. The method of any one of claims 37 to 39, wherein the anti-fungal agent comprises posaconazole.

41. The method of claim 37, wherein the anti-fungal agent comprises LAmB (liposomal amphotericin B).


SEQUENCE LISTING (56 sequences)

SEQ ID NO: 1 - protein, 12 aa, Artificial Sequence
  Description of Artificial Sequence Synthetic peptide
QSLLDSDGKTFL

SEQ ID NO: 2 - protein, 11 aa, Artificial Sequence
  Description of Artificial Sequence Synthetic peptide
QSLLYSNGKTY

SEQ ID NO: 3 - protein, 11 aa, Artificial Sequence
  Description of Artificial Sequence Synthetic peptide
QSLLDSDGKTF

SEQ ID NO: 4 - protein, 3 aa, Artificial Sequence
  Description of Artificial Sequence Synthetic peptide
LVS

SEQ ID NO: 5 - protein, 3 aa, Artificial Sequence
  Description of Artificial Sequence Synthetic peptide
QVS

SEQ ID NO: 6 - protein, 9 aa, Artificial Sequence
  Description of Artificial Sequence Synthetic peptide
WQGTHFPHT

SEQ ID NO: 7 - protein, 9 aa, Artificial Sequence
  Description of Artificial Sequence Synthetic peptide
LQGTYFPHT

SEQ ID NO: 8 - protein, 110 aa, Artificial Sequence
  Description of Artificial Sequence Synthetic polypeptide
DVVMTQTPLSLSVTIGQPASISCKSSQSLLYSNGKTYLNWLQQRPGQAPKLLMFQVSKPGIPDRFSGSGSETDFTLKISRVEAEDLGVYYCLQGTYFPHTFGAGTKVEIK

SEQ ID NO: 9 - protein, 110 aa, Artificial Sequence
  Description of Artificial Sequence Synthetic polypeptide
DVVMTQTPLTLSVTIGQPASISCKSSQSLLDSDGKTFLNWLLQRPGQSPKRLIYLVSKSGVPDRFTGTGSGTDFTLKISRVEAEDLGVYYCWQGTHFPHTFGAGTKVEIK

SEQ ID NO: 10 - protein, 110 aa, Artificial Sequence; identical to SEQ ID NO: 8
  Description of Artificial Sequence Synthetic polypeptide

SEQ ID NO: 11 - protein, 110 aa, Artificial Sequence
  Description of Artificial Sequence Synthetic polypeptide
DIVMTQTPLSLSVTPGQPASISCKSSQSLLDSDGKTFLYWYLQKPGQSPQLLIYLVSSRFSPDRFSGSGSGTDFTLKISRVEAEDVGVYYCWQGTHFPHTFGQGTKVEIK

SEQ ID NO: 12 - protein, 112 aa, Artificial Sequence
  Description of Artificial Sequence Synthetic polypeptide
DIVMTQTPLSLSVTPGQPASISCKSSQSLLDSDGKTFLYWYLQKPGQSPQLLIYLVSKLDSGVPDRFSGSGSGTDFTLKISRVEAEDVGVYYCWQGTHFPHTFGQGTKVEIK

SEQ ID NO: 13 - protein, 112 aa, Artificial Sequence
  Description of Artificial Sequence Synthetic polypeptide
DIVMTQTPLSLSVTPGQPASISCKSSQSLLDSDGKTFLNWLLQKPGQSPQLLIYLVSKLDSGVPDRFSGSGSGTDFTLKISRVEAEDVGVYYCWQGTHFPHTFGQGTKVEIK

SEQ ID NO: 14 - protein, 112 aa, Artificial Sequence
  Description of Artificial Sequence Synthetic polypeptide
DIVMTQTPLSLSVTPGQPASISCKSSQSLLDSDGKTFLNWLLQKPGQSPQRLIYLVSKLDSGVPDRFSGSGSGTDFTLKISRVEAEDVGVYYCWQGTHFPHTFGQGTKVEIK

SEQ ID NO: 15 - protein, 217 aa, Artificial Sequence
  Description of Artificial Sequence Synthetic polypeptide
DVVMTQTPLTLSVTIGQPASISCKSSQSLLDSDGKTFLNWLLQRPGQSPKRLIYLVSKSGVPDRFTGTGSGTDFTLKISRVEAEDLGVYYCWQGTHFPHTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 16 - protein, 217 aa, Artificial Sequence
  Description of Artificial Sequence Synthetic polypeptide
DVVMTQTPLSLSVTIGQPASISCKSSQSLLYSNGKTYLNWLQQRPGQAPKLLMFQVSKPGIPDRFSGSGSETDFTLKISRVEAEDLGVYYCLQGTYFPHTFGAGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 17 - protein, 217 aa, Artificial Sequence
  Description of Artificial Sequence Synthetic polypeptide
DIVMTQTPLSLSVTPGQPASISCKSSQSLLDSDGKTFLYWYLQKPGQSPQLLIYLVSSRFSPDRFSGSGSGTDFTLKISRVEAEDVGVYYCWQGTHFPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 18 - protein, 219 aa, Artificial Sequence
  Description of Artificial Sequence Synthetic polypeptide
DIVMTQTPLSLSVTPGQPASISCKSSQSLLDSDGKTFLYWYLQKPGQSPQLLIYLVSKLDSGVPDRFSGSGSGTDFTLKISRVEAEDVGVYYCWQGTHFPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 19 - protein, 219 aa, Artificial Sequence
  Description of Artificial Sequence Synthetic polypeptide
DIVMTQTPLSLSVTPGQPASISCKSSQSLLDSDGKTFLNWLLQKPGQSPQLLIYLVSKLDSGVPDRFSGSGSGTDFTLKISRVEAEDVGVYYCWQGTHFPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 20 - protein, 219 aa, Artificial Sequence
  Description of Artificial Sequence Synthetic polypeptide
DIVMTQTPLSLSVTPGQPASISCKSSQSLLDSDGKTFLNWLLQKPGQSPQRLIYLVSKLDSGVPDRFSGSGSGTDFTLKISRVEAEDVGVYYCWQGTHFPHTFGQGTKVEIKRTVAAPSVFIFPPSDEQLKSGTASVVCLLNNFYPREAKVQWKVDNALQSGNSQESVTEQDSKDSTYSLSSTLTLSKADYEKHKVYACEVTHQGLSSPVTKSFNRGEC

SEQ ID NO: 21 - protein, 8 aa, Artificial Sequence
  Description of Artificial Sequence Synthetic peptide
GDSITSGY

SEQ ID NO: 22 - protein, 7 aa, Artificial Sequence
  Description of Artificial Sequence Synthetic peptide
IKYSGRT

SEQ ID NO: 23 - protein, 5 aa, Artificial Sequence
  Description of Artificial Sequence Synthetic peptide
ASRGY

SEQ ID NO: 24 - protein, 111 aa, Artificial Sequence
  Description of Artificial Sequence Synthetic polypeptide
DLQLQESGPSLVKPSQTLSLTCSVTGDSITSGYWNWIRKFPGNKLEHMGYIKYSGRTFYNPSLKSRVSITRDTSKNQYYLQLNSVTSEDTATYYCASRGYWGQGTTLTVSS

SEQ ID NO: 25 - protein, 111 aa, Artificial Sequence
  Description of Artificial Sequence Synthetic polypeptide
QVQLQESGPGLVKPSETLSLTCTVSGDSITSGYWSWIRQPPGKGLEWIGYIKYSGRTNYNPSLKSRVTISVDTSKNQFSLKLSSVTAADTAVYYCASRGYWGQGTLVTVSS

SEQ ID NO: 26 - protein, 122 aa, Artificial Sequence
  Description of Artificial Sequence Synthetic polypeptide
QVQLQESGPGLVKPSETLSLTCTVSGDSITSGYWSWIRQPPGKGLEWIGYIKYSGRTNYNPSLKSRVTISRDTSKNQFSLKLSSVTAADTAVYYCASRGYWGQGTLVTVSSASTKGPSVFPL

SEQ ID NO: 27 - protein, 122 aa, Artificial Sequence
  Description of Artificial Sequence Synthetic polypeptide
QVQLQESGPGLVKPSETLSLTCTVSGDSITSGYWSWIRQPPGKGLEHIGYIKYSGRTFYNPSLKSRVTISRDTSKNQFSLKLSSVTAADTAVYYCASRGYWGQGTLVTVSSASTKGPSVFPL

SEQ ID NO: 28 - protein, 122 aa, Artificial Sequence
  Description of Artificial Sequence Synthetic polypeptide
QVQLQESGPGLVKPSETLSLTCTVSGDSITSGYWNWIRQPPGKGLEHIGYIKYSGRTFYNPSLKSRVTISRDTSKNQFSLKLSSVTAADTAVYYCASRGYWGQGTLVTVSSASTKGPSVFPL

SEQ ID NO: 29 - protein, 441 aa, Artificial Sequence
  Description of Artificial Sequence Synthetic polypeptide
DLQLQESGPSLVKPSQTLSLTCSVTGDSITSGYWNWIRKFPGNKLEHMGYIKYSGRTFYNPSLKSRVSITRDTSKNQYYLQLNSVTSEDTATYYCASRGYWGQGTTLTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 30 - protein, 441 aa, Artificial Sequence
  Description of Artificial Sequence Synthetic polypeptide
QVQLQESGPGLVKPSETLSLTCTVSGDSITSGYWSWIRQPPGKGLEWIGYIKYSGRTNYNPSLKSRVTISVDTSKNQFSLKLSSVTAADTAVYYCASRGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 31 - protein, 441 aa, Artificial Sequence
  Description of Artificial Sequence Synthetic polypeptide
QVQLQESGPGLVKPSETLSLTCTVSGDSITSGYWSWIRQPPGKGLEWIGYIKYSGRTNYNPSLKSRVTISRDTSKNQFSLKLSSVTAADTAVYYCASRGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 32 - protein, 441 aa, Artificial Sequence
  Description of Artificial Sequence Synthetic polypeptide
QVQLQESGPGLVKPSETLSLTCTVSGDSITSGYWSWIRQPPGKGLEHIGYIKYSGRTFYNPSLKSRVTISRDTSKNQFSLKLSSVTAADTAVYYCASRGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 33 - protein, 441 aa, Artificial Sequence
  Description of Artificial Sequence Synthetic polypeptide
QVQLQESGPGLVKPSETLSLTCTVSGDSITSGYWNWIRQPPGKGLEHIGYIKYSGRTFYNPSLKSRVTISRDTSKNQFSLKLSSVTAADTAVYYCASRGYWGQGTLVTVSSASTKGPSVFPLAPSSKSTSGGTAALGCLVKDYFPEPVTVSWNSGALTSGVHTFPAVLQSSGLYSLSSVVTVPSSSLGTQTYICNVNHKPSNTKVDKKVEPKSCDKTHTCPPCPAPELLGGPSVFLFPPKPKDTLMISRTPEVTCVVVDVSHEDPEVKFNWYVDGVEVHNAKTKPREEQYNSTYRVVSVLTVLHQDWLNGKEYKCKVSNKALPAPIEKTISKAKGQPREPQVYTLPPSRDELTKNQVSLTCLVKGFYPSDIAVEWESNGQPENNYKTTPPVLDSDGSFFLYSKLTVDKSRWQQGNVFSCSVMHEALHNHYTQKSLSLSPGK

SEQ ID NO: 34 - protein, 17 aa, Unknown
  Description of Unknown CotH3 peptide sequence
MGQTNDGAYRDPTDNNK

SEQ ID NO: 35 - protein, 601 aa, Rhizopus delemar
MKLSIISAAFLVAITHAASIKFNVIAPNATDVKVSVNGQQVTLTASDANVPYFTGSAEVGASKTYKYVAGGTEESFDRSLDGITNSTLNDFYNRPVTYANLPQLPWPIEKDPQWTRSGSKADIFDDNYIPSVFFHGDDSQVQNVVKNVPADRISGTLTFIGSNYVYSFQNVSFGIHGAGKKHNNAKQSWNWILSGSDTMGNRNFFKLRHMEEDPTQIRERLYSDILHAMGTYANDATMVRLFINNQGFGTFNMLDDITQFSYINAKFYNGKPPATLGPLYDGASGADFLYHPGNLDGYSSWVANTANPNGEAYEALDPLCKAWNETTYTDNTAIANFEKMFDLDRFMRFMVIEYLTADWDGYWMGQTNDGAYRDPTDNNKWYFLDQDFDGTFGVNLAAPEGNAFLDVSYKDFPSRYPGAVMINNLLQNADKKATFEKYLTETVRVLFNNVTLTNRVLALHNFLLPDLEWDRSIVQQSPGINFGWTFDQVTQNLWQGVTAPNNNGGGAAFGLVEYIAAKAQAVAKEFNISIVSQPVGPPSANGTTAAAPAPAAGNSTGKGGNQSISSSASSNKTSAQSTSGASRSKTAPIVLAISALALLVF

SEQ ID NO: 36 - protein, 601 aa, Rhizopus oryzae; identical to SEQ ID NO: 35

SEQ ID NO: 37 - protein, 603 aa, Lichtheimia corymbifera
MKLSIISAAFLVAITHAASIKFNVIAPNATDVKVSVNGQQVTLTASDANVPYFTGSAEVGSSKTYKYVAGGTEESFDRSLDGITNSTLNDFYNRPVTYANLPQLPWPIEKDPQWTRSGNKADIFDDNYIPSVFFHGDDSQVQNVVKNVPADRISGTLTFIGSNYVYSFQNVSFGIHGAGKKHNNAKQSWNWILSGSDTMGNRNFFKLRHMEEDPTQIRERLYSDILHAMGTYANDATMVRLFINNQGFGTFNMLDDITQFSYINAKFYNGKPPATLGPLYDGASGADFLYHPGNLDGYSSWVANTANPNGEAYEALDPLCKAWNETTYTDNTAIANFEKMFDLDRFMRFMVIEYLTADWDGYWMGQTNDGAYRDPTDNNKWYFLDQDFDGTFGVNLAAPEGNAFLDVSYKDFPSRYPGAVMINNLLQNADKKATYEKYLTETVRVLFNNVTLTNRVLALHNFLLPDLEWDRSIVQQSPGINFGWTFDQVTQNLWQGVTAPNNNGGGAAFGLVEYIATKAQAVAKEFNISIVSQPVGPPSANGTTAAAPAPAAGNSTGKGGNQSISSSASSNKTSAQSTSGASRSKTAPIIFSHFRFSSPLYSK

SEQ ID NO: 38 - protein, 19 aa, Artificial Sequence
  Description of Artificial Sequence Synthetic peptide
METGLRWLLLVAVLKGVQC

SEQ ID NO: 39 - protein, 22 aa, Artificial Sequence
  Description of Artificial Sequence Synthetic peptide
MDTRAPTQLLGLLLLWLPGSRC

SEQ ID NO: 40 - protein, 113 aa, Artificial Sequence
  Description of Artificial Sequence Synthetic polypeptide
DVVMTQTPLTLSVTIGQPASISCKSSQSLLDSDGKTFLNWLLQRPGQSPKRLIYLVSKLDSGVPDRFTGTGSGTDFTLKISRVEAEDLGVYYCWQGTHFPHTFGAGTKLELKR

SEQ ID NO: 41 - DNA, 327 bp, Artificial Sequence
  Description of Artificial Sequence Synthetic polynucleotide
cagcttcaggagtcaggacctagcctcgtgaaaccttctcagactctgtccctcacctgttctgtcactggcgactccatcaccagtggttactggaactggatccggaaattcccagggaataaacttgaacacatggggtacataaagtacagtggtcgcactttctacaatccatctctcaaaagtcgagtctccatcactcgagacacatccaagaaccagtactacctgcagttgaattctgtgacttctgaggacacagccacatattattgtgcaagccgcggctactggggccaaggcaccactctcacagtctcctca

SEQ ID NO: 42 - DNA, 339 bp, Artificial Sequence
  Description of Artificial Sequence Synthetic polynucleotide
gatgttgtgatgacccagactccactcactttgtcggttaccattggacaaccagcctccatctcttgcaagtcaagtcagagcctcttagatagtgacggaaagacatttttgaattggttgttacagaggccaggccagtctccaaagcgcctaatctatctggtgtctaaactggactctggagtccctgacaggttcactggcactggatcagggacagatttcacactgaaaatcagcagagtggaggctgaggatttgggagtttattattgctggcaaggtacacattttcctcacacgttcggtgctgggaccaagctggagctgaaacgg

SEQ ID NO: 43 - DNA, 717 bp, Artificial Sequence
  Description of Artificial Sequence Synthetic polynucleotide
atggaaaccgggctgagatggctgctgctggtcgctgtgcttaagggagtccagtgtgacatcgtgatgacccagacacctctgagcctgagcgtgacacctggacagcctgccagcatcagctgcaagtctagccagagcctgctggactccgacggcaagaccttcctgtactggtatctgcagaagcccggccagtctcctcagctgctgatctacctggtgtccaagctggatagcggcgtgcccgatagattttctggctctggcagcggcaccgacttcaccctgaagatctctagagtggaagccgaggacgtgggcgtgtactactgttggcagggcacacacttccctcacacctttggacagggtaccaaggtcgagatcaaacgcacagtggccgctccatctgtcttcatttttccacccagtgacgaacagctgaaaagcgggactgcctccgtggtctgtctgctgaacaatttctacccccgggaagccaaggtgcagtggaaagtcgataacgctctgcagtcaggcaatagccaggagtccgtgaccgaacaggactctaaggatagtacatattcactgagttcaactctgaccctgtccaaagcagactacgagaagcataaagtgtatgcctgtgaagtcacccaccaggggctgtcctcaccagtcactaagtccttcaataggggcgaatgctga

SEQ ID NO: 44 - DNA, 1392 bp, Artificial Sequence
  Description of Artificial Sequence Synthetic polynucleotide
atggatacaagggctcctactcagctgctgggcctgctgctgctgtggctgcctggatccagatgtcaagtgcagctccaagagtctggccctggcctggtcaagcctagcgaaacactgagcctgacctgtaccgtgtccggcgatagcatcaccagcggctactggtcctggatcagacagcctcctggcaaaggcctggaatggatcggctacatcaagtacagcggccggaccaactacaaccccagcctgaagtccagagtgaccatcagccgggacaccagcaagaaccagttctccctgaagctgagcagcgtgacagccgccgatacagccgtgtactactgtgccagcagaggctattggggccagggcacactggtcacagtgtctagcgccagcacaaaggggccctctgtgttccctctggcccccagcagcaagagcacatctggcggaacagccgccctgggctgcctggtgaaagactacttccccgagcccgtgaccgtgtcctggaactctggcgccctgaccagcggcgtgcacacctttccagccgtgctgcagagcagcggcctgtacagcctgagcagcgtggtgacagtgcccagcagcagcctgggcacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagccccaggtgtacacactgccccccagccgggacgagctgaccaagaaccaggtgtccctgacctgtctggtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagtga

SEQ ID NO: 45 - DNA, 717 bp, Artificial Sequence
  Description of Artificial Sequence Synthetic polynucleotide
atggaaaccgggctgagatggctgctgctggtcgctgtgcttaagggagtccagtgtgacatcgtgatgacccagacacctctgagcctgagcgtgacacctggacagcctgccagcatcagctgcaagtctagccagagcctgctggactccgacggcaagaccttcctgaactggctgctgcagaagcccggccagtctcctcagctgctgatctacctggtgtccaagctggatagcggcgtgcccgatagattttctggctctggcagcggcaccgacttcaccctgaagatctctagagtggaagccgaggacgtgggcgtgtactactgttggcagggcacacacttccctcacacctttggacagggtaccaaggtcgagatcaaacgcacagtggccgctccatctgtcttcatttttccacccagtgacgaacagctgaaaagcgggactgcctccgtggtctgtctgctgaacaatttctacccccgggaagccaaggtgcagtggaaagtcgataacgctctgcagtcaggcaatagccaggagtccgtgaccgaacaggactctaaggatagtacatattcactgagttcaactctgaccctgtccaaagcagactacgagaagcataaagtgtatgcctgtgaagtcacccaccaggggctgtcctcaccagtcactaagtccttcaataggggcgaatgctga

SEQ ID NO: 46 - DNA, 1392 bp, Artificial Sequence
  Description of Artificial Sequence Synthetic polynucleotide
atggatacaagggctcctactcagctgctgggcctgctgctgctgtggctgcctggatccagatgtcaagtgcagctccaagagtctggccctggcctggtcaagcctagcgaaacactgagcctgacctgtaccgtgtccggcgatagcatcaccagcggctactggtcctggatcagacagcctcctggcaaaggcctggaacacatcggctacatcaagtacagcggccggaccttttacaaccccagcctgaagtccagagtgaccatcagccgggacaccagcaagaaccagttctccctgaagctgagcagcgtgacagccgccgatacagccgtgtactactgtgccagcagaggctattggggccagggcacactggtcacagtgtctagcgccagcacaaaggggccctctgtgttccctctggcccccagcagcaagagcacatctggcggaacagccgccctgggctgcctggtgaaagactacttccccgagcccgtgaccgtgtcctggaactctggcgccctgaccagcggcgtgcacacctttccagccgtgctgcagagcagcggcctgtacagcctgagcagcgtggtgacagtgcccagcagcagcctgggcacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagccccaggtgtacacactgccccccagccgggacgagctgaccaagaaccaggtgtccctgacctgtctggtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagtga

SEQ ID NO: 47 - DNA, 717 bp, Artificial Sequence
  Description of Artificial Sequence Synthetic polynucleotide
atggaaaccgggctgagatggctgctgctggtcgctgtgcttaagggagtccagtgtgacatcgtgatgacccagacacctctgagcctgagcgtgacacctggacagcctgccagcatcagctgcaagtctagccagagcctgctggactccgacggcaagaccttcctgaactggctgctgcagaagcccggccagtctcctcagcggctgatctacctggtgtccaagctggatagcggcgtgcccgatagattttctggctctggcagcggcaccgacttcaccctgaagatctctagagtggaagccgaggacgtgggcgtgtactactgttggcagggcacacacttccctcacacctttggacagggtaccaaggtcgagatcaaacgcacagtggccgctccatctgtcttcatttttccacccagtgacgaacagctgaaaagcgggactgcctccgtggtctgtctgctgaacaatttctacccccgggaagccaaggtgcagtggaaagtcgataacgctctgcagtcaggcaatagccaggagtccgtgaccgaacaggactctaaggatagtacatattcactgagttcaactctgaccctgtccaaagcagactacgagaagcataaagtgtatgcctgtgaagtcacccaccaggggctgtcctcaccagtcactaagtccttcaataggggcgaatgctga

SEQ ID NO: 48 - DNA, 1392 bp, Artificial Sequence
  Description of Artificial Sequence Synthetic polynucleotide
atggatacaagggctcctactcagctgctgggcctgctgctgctgtggctgcctggatccagatgtcaagtgcagctccaagagtctggccctggcctggtcaagcctagcgaaacactgagcctgacctgtaccgtgtccggcgatagcatcaccagcggctactggaattggatcagacagcctcctggcaaaggcctggaacacatcggctacatcaagtacagcggccggaccttttacaaccccagcctgaagtccagagtgaccatcagccgggacaccagcaagaaccagttctccctgaagctgagcagcgtgacagccgccgatacagccgtgtactactgtgccagcagaggctattggggccagggcacactggtcacagtgtctagcgccagcacaaaggggccctctgtgttccctctggcccccagcagcaagagcacatctggcggaacagccgccctgggctgcctggtgaaagactacttccccgagcccgtgaccgtgtcctggaactctggcgccctgaccagcggcgtgcacacctttccagccgtgctgcagagcagcggcctgtacagcctgagcagcgtggtgacagtgcccagcagcagcctgggcacccagacctacatctgcaacgtgaaccacaagcccagcaacaccaaggtggacaagaaggtggaacccaagagctgcgacaagacccacacctgtcccccctgccctgcccctgaactgctgggcggacccagcgtgttcctgttccccccaaagcccaaggacaccctgatgatcagccggacccccgaagtgacctgcgtggtggtggacgtgtcccacgaggaccctgaagtgaagttcaattggtacgtggacggcgtggaagtgcacaacgccaagaccaagcccagagaggaacagtacaacagcacctaccgggtggtgtccgtgctgaccgtgctgcaccaggactggctgaacggcaaagagtacaagtgcaaggtgtccaacaaggccctgcctgcccccatcgagaaaaccatcagcaaggccaagggccagccccgcgagccccaggtgtacacactgccccccagccgggacgagctgaccaagaaccaggtgtccctgacctgtctggtgaaaggcttctaccccagcgatatcgccgtggaatgggagagcaacggccagcccgagaacaactacaagaccaccccccctgtgctggacagcgacggctcattcttcctgtacagcaagctgaccgtggacaagagccggtggcagcagggcaacgtgttcagctgcagcgtgatgcacgaggccctgcacaaccactacacccagaagtccctgagcctgagccccggcaagtga

SEQ ID NO: 49 - protein, 124 aa, Artificial Sequence
  Description of Artificial Sequence Synthetic polypeptide
LLLVAVLKGVQCDIVMTQTPLSLSVTPGQPASISCKSSQSLLDSDGKTFLYWYLQKPGQSPQLLIYLVSSRFSGVPDRFSGSGSGTDFTLKISRVEAEDVGVYYCWQGTHFPHTFGQGTKVEIK

SEQ ID NO: 50 - protein, 124 aa, Artificial Sequence
  Description of Artificial Sequence Synthetic polypeptide
LLLVAVLKGVQCDIVMTQTPLSLSVTPGQPASISCKSSQSLLDSDGKTFLYWYLQKPGQSPQLLIYLVSKLDSGVPDRFSGSGSGTDFTLKISRVEAEDVGVYYCWQGTHFPHTFGQGTKVEIK

SEQ ID NO: 51 - protein, 124 aa, Artificial Sequence
  Description of Artificial Sequence Synthetic polypeptide
LLLVAVLKGVQCDIVMTQTPLSLSVTPGQPASISCKSSQSLLDSDGKTFLNWLLQKPGQSPQLLIYLVSKLDSGVPDRFSGSGSGTDFTLKISRVEAEDVGVYYCWQGTHFPHTFGQGTKVEIK

SEQ ID NO: 52 - protein, 124 aa, Artificial Sequence
  Description of Artificial Sequence Synthetic polypeptide
LLLVAVLKGVQCDIVMTQTPLSLSVTPGQPASISCKSSQSLLDSDGKTFLNWLLQKPGQSPQRLIYLVSKLDSGVPDRFSGSGSGTDFTLKISRVEAEDVGVYYCWQGTHFPHTFGQGTKVEIK

SEQ ID NO: 53 - protein, 133 aa, Artificial Sequence
  Description of Artificial Sequence Synthetic polypeptide
LLLLWLPGSRCQVQLQESGPGLVKPSETLSLTCTVSGDSITSGYWSWIRQPPGKGLEWIGYIKYSGRTNYNPSLKSRVTISVDTSKNQFSLKLSSVTAADTAVYYCASRGYWGQGTLVTVSSASTKGPSVFPL

SEQ ID NO: 54 - protein, 133 aa, Artificial Sequence
  Description of Artificial Sequence Synthetic polypeptide
LLLLWLPGSRCQVQLQESGPGLVKPSETLSLTCTVSGDSITSGYWSWIRQPPGKGLEWIGYIKYSGRTNYNPSLKSRVTISRDTSKNQFSLKLSSVTAADTAVYYCASRGYWGQGTLVTVSSASTKGPSVFPL

SEQ ID NO: 55 - protein, 133 aa, Artificial Sequence
  Description of Artificial Sequence Synthetic polypeptide
LLLLWLPGSRCQVQLQESGPGLVKPSETLSLTCTVSGDSITSGYWSWIRQPPGKGLEHIGYIKYSGRTFYNPSLKSRVTISRDTSKNQFSLKLSSVTAADTAVYYCASRGYWGQGTLVTVSSASTKGPSVFPL

SEQ ID NO: 56 - protein, 133 aa, Artificial Sequence
  Description of Artificial Sequence Synthetic polypeptide
LLLLWLPGSRCQVQLQESGPGLVKPSETLSLTCTVSGDSITSGYWNWIRQPPGKGLEHIGYIKYSGRTFYNPSLKSRVTISRDTSKNQFSLKLSSVTAADTAVYYCASRGYWGQGTLVTVSSASTKGPSVFPL